SEQUENCE LISTING (48 sequences)

SEQ ID NO: 1 - DNA, 20 bp, synthetic construct
  source 1..20: mol_type = other DNA
agtgtgtactcagagcggtg

SEQ ID NO: 2 - DNA, 20 bp, synthetic construct
  source 1..20: mol_type = other DNA
gagaactgcaatgttaacaa

SEQ ID NO: 3 - DNA, 20 bp, synthetic construct
  source 1..20: mol_type = other DNA
gccggcccttgccatacacg

SEQ ID NO: 4 - DNA, 20 bp, synthetic construct
  source 1..20: mol_type = other DNA
tcctcactcaccggttccgg

SEQ ID NO: 5 - DNA, 91 bp, synthetic construct
  source 1..91: mol_type = other DNA
aatgatacggcgaccaccgagatctacacctgatgacactctttccctacacgacgctcttccgatctnttgtggaaaggacgaaacaccg

SEQ ID NO: 6 - DNA, 91 bp, synthetic construct
  source 1..91: mol_type = other DNA
caagcagaagacggcatacgagatnnnnnnnngtgactggagttcagacgtgtgctcttccgatctccaattcccactcctttcaagacct

SEQ ID NO: 7 - DNA, 20 bp, synthetic construct
  source 1..20: mol_type = other DNA
aggtgcattggtctgcaaat

SEQ ID NO: 8 - DNA, 20 bp, synthetic construct
  source 1..20: mol_type = other DNA
tctctgctaatcgctcaacg

SEQ ID NO: 9 - DNA, 20 bp, synthetic construct
  source 1..20: mol_type = other DNA
aggtgcattggtctgcaaat

SEQ ID NO: 10 - DNA, 20 bp, synthetic construct
  source 1..20: mol_type = other DNA
tctctgctaatcgctcaacg

SEQ ID NO: 11 - DNA, 20 bp, synthetic construct
  source 1..20: mol_type = other DNA
aggcaataaacacatttgga

SEQ ID NO: 12 - DNA, 20 bp, synthetic construct
  source 1..20: mol_type = other DNA
caagtcgaggcagacattca

SEQ ID NO: 13 - DNA, 22 bp, synthetic construct
  source 1..22: mol_type = other DNA
cagggtgtcaaggatggataaa

SEQ ID NO: 14 - DNA, 24 bp, synthetic construct
  source 1..24: mol_type = other DNA
tggtccattcctcatgcatccgac

SEQ ID NO: 15 - DNA, 939 bp, synthetic construct
  source 1..939: mol_type = other DNA
atggactacaaggacgacgatgacaagggatccgacagatgtgagcgaaatgagttccagtgccaagatggaaaatgcatatcctacaagtgggtctgtgatggttcagcggagtgccaggatggctctgacgagtcccaagaaacgtgtctttctgtgacttgtaagagcggcgattttagctgcggcggcagggtcaacaggtgtattccccagttttggcgctgcgacgggcaggttgactgcgacaatggaagcgacgagcagggttgtccgccaaaaacctgtagtcaggatgaatttagatgccacgatggaaaatgtatatctcgacaatttgtgtgcgactctgaccgagactgcctggacggtagtgacgaagcgtcctgccccgtattgacatgtggaccggccagctttcagtgtaactctagcacctgtattcctcaactgtgggcttgtgataatgatcctgactgcgaagatgggtcagacgaatggcctcagagatgccggggactttatgtttttcaaggtgattcctccccttgcagcgcatttgaatttcactgcctctcaggtgagtgcatacatagcagctggcgctgcgacggggggcctgattgcaaggataagtctgacgaagagaactgcgctgtagctacctgtaggccagatgaattccagtgctcagacgggaattgtattcacggtagtcgccagtgtgaccgcgagtacgattgcaaagacatgtcagacgaggtaggctgcgtaaatgttacgctttgcgagggaccaaacaaatttaagtgccattccggtgaatgcataacgctggacaaggtctgcaacatggcgagagattgtcgcgattggagcgacgaacctattaaggagtgtggaggatcccatataaacgaatgcttggtaaataatggt

SEQ ID NO: 16 - DNA, 330 bp, synthetic construct
  source 1..330: mol_type = other DNA
atggactacaaggacgacgatgacaagggatccaaaacgtgctcacccaagcaattcgcatgccgcgaccaaatcacttgtatatccaagggatggaggtgtgacggtgagcgagactgccccgatggttccgacgaggcgcctgagatctgtccacaatctaaagcccaacgctgccaacctaacgagcacaattgcttgggtactgaactttgcgtccccatgtctcgcctttgtaatggtgtccaagattgcatggacgggagcgacgaggggccacattgtagagaaggaggatcccatataaacgaatgcttggtaaataatggt

SEQ ID NO: 17 - DNA, 1065 bp, synthetic construct
  source 1..1065: mol_type = other DNA
atggactacaaggacgacgatgacaagggatccccgcagtgccagccaggcgaattcgcttgtgccaactctcgctgtatacaggagcgctggaaatgtgatggcgataacgattgcttggataattccgatgaggcaccagcgctttgtcatcaacatacatgccccagcgatcgctttaaatgcgaaaacaacagatgcatccccaacaggtggctttgtgatggtgataacgattgcggtaactctgaagatgaatccaacgctacctgttctgcgcggacgtgtccgcccaaccaattctcctgtgcatccggccgatgtattcctatctcatggacttgtgatcttgacgacgattgtggcgatcgatctgatgaatccgcgagctgtgcatatccaacgtgtttcccgctgacgcaattcacttgcaataatggcaggtgtatcaatattaattggcgatgcgataatgacaatgactgtggtgataactctgatgaagcaggctgcagccactcctgttcttcaactcagtttaaatgcaattcaggaaggtgtatcccagaacattggacttgtgatggcgacaacgattgcggcgactattctgatgaaacacacgcgaactgcacgaatcaggcgacgcgaccccccggcgggtgtcacacggacgaattccagtgtcgcctggatggcttgtgtattccgctccgatggcgatgtgacggcgacaccgattgcatggattcctccgatgaaaaatcctgcgaaggcgtgacacacgtttgcgacccgagtgtgaaattcggctgtaaggacagcgcccggtgtattagcaaggcgtgggtgtgcgatggggacaatgactgcgaggacaattctgatgaagagaattgcgagtctttggcatgtcgccccccgtctcatccatgcgccaacaatacatctgtctgtttgccccctgacaagctttgtgacggaaatgacgactgcggcgacgggtcagacgaaggggaattgtgtggaggatcccatataaacgaatgcttggtaaataatggt

SEQ ID NO: 18 - DNA, 1329 bp, synthetic construct
  source 1..1329: mol_type = other DNA
atggactacaaggacgacgatgacaagggatccagttcctgtcgagcgcaggatgagttcgaatgcgcgaatggagagtgtattaattttagtctcacttgcgatggcgtaccccattgtaaagacaaaagcgatgaaaaaccttcatactgcaattcccggcgatgcaagaaaaccttccggcagtgttctaatggtaggtgcgtgtcaaacatgctttggtgcaacggagcggacgattgcggcgatggatccgacgagatcccttgtaataagaccgcatgcggtgtcggcgagtttcggtgtcgggatggaacctgtataggaaactcttctcggtgcaatcagttcgttgactgtgaagacgcgtccgatgaaatgaactgctccgccaccgactgtagctcatattttcgactcggagtgaaaggtgtgctctttcaaccgtgtgaacgcaccagtctgtgctatgccccctcttgggtctgtgatggagctaacgattgcggcgattacagtgatgagcgcgactgtcccggggtcaaacgcccccgctgtcccttgaactattttgcatgtccttcaggcagatgtattccgatgtcatggacttgcgacaaagaggatgactgtgaacatggagaggacgaaacgcactgtaacaagttctgctccgaagctcaattcgaatgtcaaaaccacaggtgtatcagtaaacagtggctttgtgacggtagtgatgactgcggggatggatctgatgaagcagcccattgcgagggtaagacatgtgggccttcttccttctcttgtccaggcacgcatgtctgtgtcccagagagatggctctgcgacggagataaggattgtgcagacggggctgatgaatctatcgctgcaggatgtctctataatagtacctgtgatgacagagaattcatgtgccaaaatcgacagtgcattcccaagcactttgtctgcgatcacgaccgagactgtgccgacgggtctgatgaatctcccgagtgtgaatacccaacctgtggcccatcagaatttcgatgcgctaacggtcgctgcctttcttctcggcagtgggaatgcgatggggagaacgactgtcacgaccaaagtgatgaggctcctaaaaatccacactgcacgagtcaagaacataaatgcaatgcatcaagtcagtttctttgtagttctggccggtgcgtggcagaagcgctgctttgcaacggacaagatgattgcggagatagcagcgacgaaaggggatgtcatggaggatcccatataaacgaatgcttggtaaataatggt

SEQ ID NO: 19 - DNA, 1410 bp, synthetic construct
  source 1..1410: mol_type = other DNA
atggactacaaggacgacgatgacaagggatcctcaaattgtactgcctcccagttcgtgtgcaagaatgataagtgtatcccattctggtggaaatgtgatacagaggatgactgcggggatcattccgacgagcccccggattgcccggaatttaagtgtcgcccagggcaatttcagtgtagtactggtatatgcactaacccagccttcatatgtgatggagataatgactgccaagataactctgacgaggcgaactgcgatatacatgtatgtcttccgagtcagttcaagtgcactaatacgaatcgctgtattccaggcatctttagatgtaatggtcaggacaactgcggcgatggagaggatgagagagactgcccagaggtgacttgcgctcctaatcagtttcaatgtagcataaccaaaagatgtatcccgcgagtttgggtgtgtgacagggacaatgactgcgtcgatggaagtgatgagcccgcgaactgcacccaaatgacgtgtggtgtagacgagtttcgctgtaaggacagcgggagatgtatcccggcccgatggaagtgcgatggtgaagacgattgcggtgatggatcagatgagcctaaggaggagtgcgacgaacgcacttgcgagccgtaccaattccggtgcaagaacaacaggtgtgtacccggccgctggcagtgtgactatgataacgattgtggggacaatagcgacgaagagtcttgtactcccaggccatgttctgaatccgagttctcatgtgccaatggaaggtgtatcgccgggcggtggaaatgtgacggtgaccatgactgcgcggatggctccgatgaaaaagattgtacccctcggtgcgatatggaccagttccagtgtaaatccggacattgtataccattgcggtggaggtgtgacgctgatgcagactgtatggatggtagtgatgaagaggcctgcggtacaggtgttcgcacatgccctttggacgagttccaatgcaataacaccctttgtaagccgctggcttggaaatgcgatggcgaagatgattgtggtgataattccgatgagaatcctgaagaatgtgctaggttcgtttgcccgcctaacagaccttttcggtgtaagaacgaccgggtttgcttgtggataggacggcagtgtgatggtacggacaactgtggcgacggtactgacgaagaagactgcgagcctccaaccgcccatacgactcactgtaaagataagaaggagtttctttgccgcaaccaacgctgtcttagctcttcactgcggtgtaatatgttcgacgactgcggtgacggcagtgatgaggaagactgtagtggaggatcccatataaacgaatgcttggtaaataatggt

SEQ ID NO: 20 - DNA, 408 bp, synthetic construct
  source 1..408: mol_type = other DNA
atggactacaaggacgacgatgacaagggatccgaatttttgttggcccttttgaccctgagcggtttgctgccaatagcgcgcgttttgactgtaggagcagatagggaccagcaactgtgtgaccctggggagttcctttgccatgatcacgtgacgtgtgtttcccaaagttggctgtgtgacggcgacccagactgccccgatgacagtgatgagagcctcgatacatgcccggaggaggtagaaataaagtgcccccttaaccatatagcttgtttgggaaccaacaaatgtgtgcatttgtcccaactgtgtaacggggtgcttgattgccccgatggttatgatgaaggtgtccactgccagggaggatcccatataaacgaatgcttggtaaataatggt

SEQ ID NO: 21 - DNA, 1065 bp, synthetic construct
  source 1..1065: mol_type = other DNA
atggactacaaggacgacgatgacaagggatccatctgcaaagcaggtgagtttcgctgtaagaaccgccattgcatacaagctcggtggaaatgcgatggggacgacgactgtctcgacggtagcgacgaggacagcgtaaactgttttaaccattcttgtcctgatgatcaattcaagtgtcaaaataatcgctgcattccaaaaaggtggttgtgtgacggcgccaacgactgtggcagcaatgaagatgagagcaatcagacatgcactgctaggacgtgtcaagtcgaccaattttcatgcggtaacggccgctgcatcccaagggcgtggctctgcgacagagaggatgactgcggagatcagaccgatgagatggcatcttgcgaatttccaacttgcgaaccactcactcaattcgtttgtaagtctggacgatgcataagttcaaaatggcactgcgatagcgatgatgattgcggagatggatcagacgaagtaggctgcgtccatagctgtttcgataatcagtttaggtgcagctcagggcggtgcattcctggccactgggcctgtgatggcgataatgattgcggcgatttcagcgatgaagcgcagattaattgtacgaaggaggagatacactcacccgcggggtgtaacgggaatgagttccaatgccacccagatggaaattgtgttcccgacctgtggcggtgtgacggagagaaagattgcgaagacggtagcgatgaaaaaggctgtaatggaaccatcagactgtgcgaccataaaactaagttttcttgctggagcacaggccgatgtatcaacaaagcttgggtctgcgacggcgatatagactgcgaagatcaaagtgacgaggatgattgcgactcctttctgtgcggtccaccaaagcatccttgtgctaacgatacgtcagtttgtcttcaaccggaaaagttgtgtaatgggaagaaagattgtccagatggctccgatgagggttatctttgcgatggaggatcccatataaacgaatgcttggtaaataatggt

SEQ ID NO: 22 - DNA, 1323 bp, synthetic construct
  source 1..1323: mol_type = other DNA
atggactacaaggacgacgatgacaagggatccagctgcaatgcgtacagcgagttcgagtgtggaaatggcgaatgcatagactatcagctgacttgtgacggaataccgcattgtaaagacaagtctgacgagaagcttctgtactgcgaaaaccggtcatgtcgccgcggttttaagccgtgctataacaggaggtgtatccctcacggaaagctgtgcgacggtgaaaatgattgtggagataactcagatgaacttgattgcaaagtgagtacgtgtgcgacggtggaattcagatgtgcggatggcacgtgcatcccccggtcagcgagatgcaaccaaaacattgactgtgcagacgcgtcagatgaaaagaactgcaataacactgattgtactcatttttataaacttggcgtcaaaaccacggggttcatacgatgcaactctaccagcctctgtgtactgcccacatggatctgtgatggcagtaacgattgcggagactattctgacgaactcaaatgtccggtgcaaaataaacacaagtgtgaagaaaactatttttcctgtccatccggccggtgcatccttaatacttggatttgcgatggtcaaaaggactgcgaggacggtcgggatgagtttcactgcgacagctcctgttcctggaaccaattcgcatgttccgcacaaaagtgcatcagtaagcattggatctgtgacggagaagatgattgtggagatggtcttgatgaatctgactcaatctgcggggccattacttgcgccgcagacatgttcagttgtcaaggttccagagcatgcgtcccacgacactggctttgtgatggagaaagggactgccctgatggtagcgatgaactgtcaaccgccgggtgcgcacctaacaacacctgtgacgaaaatgcttttatgtgccacaataaagtctgtatacccaagcagtttgtatgtgatcacgatgacgattgcggagacggatctgacgaaagtccccagtgtggctaccgacaatgcggtacggaagagttctcttgcgctgatggccgctgcctcctcaacacgcaatggcagtgcgacggtgatttcgattgtccagatcactctgacgaagccccacttaacccaaaatgcaaatccgcggaacagtcatgtaatagttcattctttatgtgtaagaacggcaggtgtataccgagtggcggactctgcgacaataaagacgattgcggggatggatccgacgagcggaattgcggaggatcccatataaacgaatgcttggtaaataatggt

SEQ ID NO: 23 - DNA, 1818 bp, synthetic construct
  source 1..1818: mol_type = other DNA
atggactacaaggacgacgatgacaagggatccggtgttatcgcgctgactctctttgaggactatatttattggaccgacggaaaaacaaaaagtttgtcccgagcccacaagacgtcaggtgcggacaggctgagtctcatctactcctggcacgccattaccgacatccaagtgtatcattcctaccgccaacctgacgtctctaaacatctctgtatgatcaacaacggcggttgtagtcatctgtgcttgctcgcgcccggcaagacgcatacctgcgcgtgccctacgaacttttatcttgcggccgataatcgcacatgcctgtccaactgcaccgcgagtcagttcaggtgcaaaaccgacaagtgcatccccttctggtggaaatgcgataccgtcgatgactgcggtgatggttctgatgagccggatgattgccctgagttcaggtgtcagccgggacgctttcaatgcggaacaggattgtgcgccctcccggcgtttatatgcgacggagaaaacgactgcggcgacaatagcgacgaactcaactgtgatacacatgtctgcctgtccggacagttcaagtgtacgaagaatcagaagtgcatcccggtcaaccttagatgcaatgggcaagacgactgtggcgatgaggaagacgaacgcgactgtcccgaaaacagttgttctcccgattattttcaatgtaagacaactaagcattgtatatcaaagctctgggtgtgtgacgaagatccagactgcgcggatgctagtgacgaggcgaactgtgacaaaaagacttgtggcccgcacgaattccaatgcaaaaataacaattgtatccctgatcattggaggtgcgactcacaaaatgattgcagcgataatagcgacgaagagaactgcaaacctcagacgtgcacactgaaggactttctgtgtgccaacggggactgtgtcagtagcagattctggtgtgatggagactttgattgcgcagatggatcagatgagagaaattgcgaaacatcttgtagcaaggaccagttcagatgttccaacggccagtgcatcccagcaaagtggaagtgtgacgggcacgaagactgcaaatacggcgaggacgaaaaatcttgtgaacctgcttcccccacatgtagcagcagggagtacatttgcgcaagtgacgggtgtatcagtgcgagtctgaaatgtaatggggagtacgactgcgcggacggttccgatgaaatggactgcgttaccgagtgcaaggaggatcaattccgatgcaagaacaaggcgcactgcatacctataaggtggctttgcgatggaattcatgactgcgtagatgggtcagacgaagaaaattgcgaacgaggtggcaacatatgcagggctgacgaatttctttgtaacaactcactgtgcaagttgcatttctgggtgtgcgatggagaagatgactgtggcgacaatagtgacgaagctcccgacatgtgcgtcaagtttctttgtccttctactcgcccacatcgctgtcggaataatcggatctgtttgcagagcgagcagatgtgtaacgggatagatgaatgtggggataatagcgatgaggatcactgcggcggaaaattgacatacaaggcgcgaccctgtaaaaaagatgagttcgcctgcagcaataaaaaatgtataccaatggacttgcaatgcgacaggcttgacgactgtggcgatggctcagatgaacaggggtgtcggggaggatcccatataaacgaatgcttggtaaataatggt

SEQ ID NO: 24 - DNA, 915 bp, synthetic construct
  source 1..915: mol_type = other DNA
atggactacaaggacgacgatgacaagggatccgaatgcgattccgcacacttccgatgcggttcaggacactgcattccggcagattggcgatgtgacgggactaaagattgttctgatgacgccgacgagatcggctgtgccgttgtgacatgtcagcagggttatttcaagtgtcaatccgagggtcagtgcataccaaatagttgggtatgtgatcaggatcaagactgtgatgacggtagtgacgaacggcaggactgctcacaatctacttgttcatctcatcagatcacgtgttccaatggccaatgcataccaagcgagtacaggtgcgaccacgtacgggactgccctgatggggcagacgaaaacgactgtcagtatcccacatgcgaacagcttacgtgtgacaatggggcatgctataacactagccagaaatgtgactggaaggtagactgtcgcgattcctcagatgagattaactgcacggagatttgtctccacaatgagttcagttgcgggaacggggagtgcataccgagggcgtacgtgtgcgatcacgacaatgactgccaagacggatcagacgagcacgcatgcaactatcccacatgtggtggttaccaattcacctgtccctctggccggtgtatataccaaaattgggtctgtgatggtgaagatgattgtaaggacaacggagatgaagatggttgcgagagcggtccacatgacgttcataaatgctccccccgcgagtggtcatgtccagaaagtggtaggtgtatttcaatctataaggtttgtgatggtatcctcgattgcccggggcgcgaggatgagaataatactagtacaggtaaatattgtggaggatcccatataaacgaatgcttggtaaataatggt

SEQ ID NO: 25 - DNA, 1044 bp, synthetic construct
  source 1..1044: mol_type = other DNA
atggactacaaggacgacgatgacaagggatcccaatgcggactctttagctttccatgtaaaaatggccggtgcgtccccaattattatctctgtgacggagtagacgactgccatgacaattccgatgagcaattgtgtggaacgttgaacaatacatgctcttcctctgccttcacctgcggtcatggcgagtgcattccggctcactggcgctgtgacaagaggaacgattgtgtcgatggtagtgatgaacataattgcccgacgcatgcgcccgcgtcatgtctcgatacgcaatacacgtgtgataatcatcaatgtatctccaagaattgggtatgcgatacggacaacgattgtggcgatgggagcgatgagaaaaattgcaattctaccgagacctgtcaacccagccagttcaattgcccaaatcaccggtgcattgacttgtccttcgtgtgtgacggtgacaaagattgcgtagacgggtctgatgaagtcgggtgtgtacttaactgcactgccagtcagttcaaatgcgcctccggggataagtgcataggcgtaacgaataggtgcgatggagtctttgactgctcagacaatagcgacgaagccggttgtccgacacgccccccggggatgtgccatagcgacgagtttcagtgtcaagaggacggcatttgcattcctaacttctgggagtgcgacggacatcccgactgcctctatggctctgacgagcataatgcgtgtgtgccaaaaacctgccctagttcttacttccactgcgacaacggcaattgtatacacagagcttggctttgtgatcgagataatgattgtggtgacatgtcagacgagaaggattgcccgactcagccgttccgctgcccatcatggcaatggcaatgtttgggccacaatatctgtgtcaatctctcagttgtctgtgacggaatttttgattgtccaaacgggacagatgaaagcccactctgtggaggatcccatataaacgaatgcttggtaaataatggt

SEQ ID NO: 26 - DNA, 1308 bp, synthetic construct
  source 1..1308: mol_type = other DNA
atggactacaaggacgacgatgacaagggatccaggtgtggggcctcttcctttacgtgctcaaacgggagatgtattagcgaggagtggaaatgcgataatgataatgactgtggtgacggaagcgatgagatggagtctgtttgtgcgttgcacacatgctcccctacagccttcacttgcgccaatggacgctgcgttcagtattcttaccgctgtgattattataatgactgtggtgacggatcagatgaagctggttgcctgttcagagactgcaacgcaaccaccgagttcatgtgcaacaatagacggtgtattccacgcgaatttatatgtaacggggtggacaactgtcacgacaataacacgtctgacgagaaaaactgtcctgatagaacctgtcaatcagggtacacgaagtgccacaactctaacatttgtattccacgagtgtacctgtgcgatggcgataatgactgtggggataactctgacgaaaacccgacgtactgtaccacccacacgtgctcatctagcgagttccagtgcgcatccggccgatgtataccacaacattggtactgtgatcaggaaacagactgcttcgatgctagcgatgagccggcttcctgtggacatagcgagagaacctgtctggcggatgagtttaagtgcgatgggggtcgctgcatcccaagcgaatggatctgtgacggggacaatgattgcggggacatgtccgatgaggacaagcggcatcagtgccagaaccagaactgctctgattccgagttcctttgcgtaaacgataggcccccggatagaagatgtatccctcaatcttgggtttgcgatggggatgttgactgcacggatggttatgatgagaatcagaactgcacccggcggacatgttccgaaaacgagttcacctgcggttacgggctctgtatacctaaaatatttcgctgcgatcgccacaacgattgtggggattatagcgacgagcggggctgcttgtatcaaacatgtcaacaaaatcaattcacatgccaaaatggtaggtgtatatccaaaacattcgtgtgcgatgaagataatgattgtggagatggttctgatgaactgatgcatctgtgccatactcctgagcccacgtgtccgccgcatgagttcaaatgcgataatggtcggtgtattgagatgatgaaattgtgtaaccacctggacgactgtctggacaactctgatgaaaagggctgtgggggaggatcccatataaacgaatgcttggtaaataatggt

SEQ ID NO: 27 - DNA, 1428 bp, synthetic construct
  source 1..1428: mol_type = other DNA
atggactacaaggacgacgatgacaagggatccatgtgcagtagcacacagttcctttgtgccaacaacgagaagtgtataccaatctggtggaaatgtgacggacagaaggattgtagcgatggatctgacgaactcgctctttgccctcaacgattctgccggctcggccaattccagtgttcagacggtaattgtacgtctccacaaaccctctgcaatgcccatcaaaactgtccggatggctccgatgaggatcgactcctgtgtgagaatcaccattgcgacagcaatgaatggcaatgtgccaacaagaggtgtatccccgaatcatggcaatgtgatacgttcaacgactgcgaagacaattcagatgaggactcatcacactgtgcctctagaacctgtcgaccggggcagtttcgctgcgctaacggccgctgtatacctcaagcatggaagtgtgacgtcgacaatgattgcggtgatcatagcgatgaaccgattgaggaatgcatgtcctccgctcacctgtgtgacaatttcaccgagttcagttgcaagaccaactacaggtgtatcccgaagtgggctgtctgtaatggtgtcgatgactgccgagataactcagacgagcaagggtgtgaggaaagaacttgccatccagtaggagattttcggtgtaagaaccaccattgtatcccgctgcgatggcaatgcgatggccaaaacgattgcggggacaacagtgacgaagaaaactgcgcgcccagagagtgtacggagtctgaatttaggtgcgttaatcaacagtgcatcccttccaggtggatctgcgaccattataatgactgtggcgataatagcgatgagcgcgactgcgagatgcgcacgtgtcatccggaatatttccaatgtacttctggtcattgcgttcattctgaacttaaatgcgacggatctgctgactgtcttgacgcctccgacgaagcagattgcccaacacgattccctgatggcgcttactgtcaggcaacaatgtttgagtgcaaaaatcatgtttgcattcctccctactggaaatgcgacggagacgatgactgcggcgatgggtctgacgaagaactgcatctgtgtttggacgtgccctgtaatagtccaaatcggtttcggtgcgataataaccgatgtatttactcccatgaagtttgcaatggcgttgacgattgtggtgacggcacggatgaaacagaagagcactgtcgcaagcccactccaaaaccatgtacggagtatgaatataaatgcggaaacggtcattgcattccgcatgacaacgtatgcgatgatgcagatgactgtggagactggtcagatgagttgggttgtggaggatcccatataaacgaatgcttggtaaataatggt

SEQ ID NO: 28 - DNA, 330 bp, synthetic construct
  source 1..330: mol_type = other DNA
atggactacaaggacgacgatgacaagggatccagctgtcaggcggacgagtttcgatgtgataatggaaaatgtttgcccgggccctggcaatgtaacaccgtagacgagtgtggggacggatctgacgaaggcaactgtagtgcccctgcgtctgagcctccaggctctctttgtccgggtgggacatttccctgcagcggggctcggtccacaaggtgtctccctgtcgaacgaaggtgtgacggacttcaagactgtggggatggttcagacgaggcgggatgtccgggaggatcccatataaacgaatgcttggtaaataatggt

SEQ ID NO: 29 - DNA, 300 bp, synthetic construct
  source 1..300: mol_type = other DNA
atggactacaaggacgacgatgacaagggatccgcttgtcccccggaccagtatccatgtgagggggggtcaggactttgctatacacccgcggataggtgtaacaatcagaagtcatgccccgatggggccgatgagaagaattgcttctcctgccaaccgggaactttccactgtggtactaatctgtgtattttcgagacgtggaggtgcgacggtcaggaagactgccaggatggttcagacgagcatgggtgtcttggaggatcccatataaacgaatgcttggtaaataatggt

SEQ ID NO: 30 - DNA, 1047 bp, synthetic construct
  source 1..1047: mol_type = other DNA
atggactacaaggacgacgatgacaagggatccgcgtgcgggcggtcacattttacctgtgcggtctcagcgttgggtgagtgtacctgtattccggcccagtggcagtgcgatggcgacaacgattgcggtgatcactctgacgaagatggctgtatccttccaacatgtagcccgctcgactttcattgtgataacggtaagtgcatccgcaggtcatgggtctgcgacggggacaacgactgcgaagacgattctgatgagcaggactgtcccccgcgcgaatgtgaggaggacgaattcccttgtcaaaatggttactgcattcgcagcctttggcactgtgacggcgacaacgactgtggggataactccgatgagcaatgcgatatgagaaagtgcagcgacaaggaattccgatgctctgatggtagctgtatagctgaacattggtactgcgatggcgatacggattgtaaggacggcagcgatgaagaaaattgtccttcagcggtcccagccccgccctgcaatctggaggaattccagtgcgcttatggccggtgcattctcgacatctaccactgcgacggtgacgacgactgcggcgactggagtgacgaaagtgactgtagttcacaccaaccttgccgatccggcgaattcatgtgtgactctggattgtgtataaatgccggttggagatgcgacggtgacgctgactgcgatgatcaatccgatgaaaggaactgtactactagtatgtgcactgccgaacagtttcgatgccactctggaagatgtgtcaggctctcttggcgatgtgatggagaagacgactgtgcagacaatagtgacgaagaaaattgtgaaaatacgggttcacctcagtgcgctctcgaccaattcctgtgttggaacgggaggtgtatagggcagcgcaaactgtgtaacggcgtcaatgactgtggggacaactctgatgagtccccacagcagaattgccgaggaggatcccatataaacgaatgcttggtaaataatggt

SEQ ID NO: 31 - DNA, 414 bp, synthetic construct
  source 1..414: mol_type = other DNA
atggactacaaggacgacgatgacaagggatccacctgctcacccgatcaattcgcctgcgccactggagaaatcgactgtattccaggagcctggcgctgtgacgggttcccggagtgcgacgaccaatcagacgaggaaggatgccctgtgtgctcagcagcgcagtttccttgcgcccgaggacaatgtgtcgatttgcggctccggtgtgatggtgaggccgactgtcaagatagatccgacgaagctgactgcgatgcgatatgtcttcccaatcagttccgctgcgcctccggacaatgtgtattgataaagcagcaatgcgattcatttccggactgtattgatggatctgacgagttgatgtgtgaaggaggatcccatataaacgaatgcttggtaaataatggt

SEQ ID NO: 32 - DNA, 414 bp, synthetic construct
  source 1..414: mol_type = other DNA
atggactacaaggacgacgatgacaagggatccacgtgctcaccacaacaatttacctgtttcactggcgaaatcgactgcatcccggttgcttggcggtgtgacggcttcactgaatgcgaggatcatagtgacgaattgaactgcccggtttgtagcgaatcacaattccaatgtgccagcggtcagtgcattgacggagccttgcggtgcaatggggacgctaactgtcaggacaaatcagacgaaaaaaattgcgaagttctgtgcctgatagatcaatttaggtgtgcgaacggccagtgcattggtaaacataagaagtgcgatcacaatgttgattgctctgacaagagcgacgagttggactgctacggaggatcccatataaacgaatgcttggtaaataatggt

SEQ ID NO: 33 - DNA, 939 bp, synthetic construct
  source 1..939: mol_type = other DNA
atggactacaaggacgacgatgacaagggatccgattgtgagaaggaccaatttcagtgtcgaaacgagagatgcatcccaagcgtatggcggtgcgatgaggacgacgattgcttggaccactcagacgaggatgactgtccaaagaaaacctgtgcagactctgattttacttgtgacaatggccattgtatacacgaacggtggaagtgcgacggtgaggaggagtgtccagacggctcagatgaaagcgaagcaacgtgtacgaaacaggtatgtcctgctgagaaattgtcatgtggacccacttctcacaaatgtgtcccggctagttggaggtgtgacggtgagaaagattgcgagggcggggctgatgaggcagggtgtgccacactctgtgcaccacatgagttccaatgcgggaaccgatcttgtctcgcagccgtatttgtttgcgatggagacgacgactgcggtgatggatctgatgaacgagggtgcgcagatccggcttgcggtccgcgcgaattcagatgtggcggtgacgggggcggagcctgtattcctgagaggtgggtctgcgatagacaattcgattgtgaggatagaagcgacgaagctgcggaactctgtggaaggccaggtccgggtgcaaccagtgcccctgctgcatgtgctactgcgagtcaattcgcgtgccgaagtggtgagtgtgtgcatcttggttggagatgcgatggggatagagattgcaaggacaaaagtgatgaagcagattgtccgctcggtacctgccgaggcgatgaattccaatgcggggacggcacctgtgtcctggcaataaaacattgtaaccaggaacaagattgccctgatggttccgatgaggcgggttgtctcggaggatcccatataaacgaatgcttggtaaataatggt

SEQ ID NO: 34 - DNA, 183 bp, synthetic construct
  source 1..183: mol_type = other DNA
atggactacaaggacgacgatgacaagggatccatgtgcctccaagaggaatttcaatgtttgaatcaccgctgcgtctccgccgttcagcgctgcgacggagtagacgcctgtggggacgggagcgatgaggcgggttgtagtggaggatcccatataaacgaatgcttggtaaataatggt

SEQ ID NO: 35 - DNA, 456 bp, synthetic construct
  source 1..456: mol_type = other DNA
atggactacaaggacgacgatgacaagggatcctactgcctgccgtgggatcgaccatgtggtttgggttcaggtctgggagcgggagagggcttgggtgaacgctgttacagtgaggcacaacgatgtgatggtagctgggactgcgctgacgggacagacgaggaagactgtcctggatgtccgccgggacacttcccctgcggagctgcaggtactagcggggccaccgcgtgttatctgcccgcagatcgctgtaactatcagacgttctgcgcagacggtgccgacgagcgccgatgtcgccattgtcaaccaggcaactttagatgtagggatgaaaagtgtgtctatgaaacgtgggtctgtgatggtcagcctgactgcgctgacggttccgacgaatgggactgttccggaggatcccatataaacgaatgcttggtaaataatggt

SEQ ID NO: 36 - DNA, 423 bp, synthetic construct
  source 1..423: mol_type = other DNA
atggactacaaggacgacgatgacaagggatccacttgctcaaggtatcactttttctgtgatgacggttgttgcatcgacataacactggcttgtgatggagtacaacagtgccccgacggaagcgacgaggacttctgtcagggcgggtctacgtgttctcgctatcactttttttgcgatgatggctgctgcatagatataactttggcgtgcgatggtgttcaacagtgtccagacgggagtgatgaagatttttgccagggtggctctacctgttctcgatatcacttcttttgcgatgatgggtgctgtatcgacataacgctggcgtgcgacggagtacaacaatgccctgacgggagcgacgaagacttttgtcagggaggatcccatataaacgaatgcttggtaaataatggt

SEQ ID NO: 37 - DNA, 345 bp, synthetic construct
  source 1..345: mol_type = other DNA
atggactacaaggacgacgatgacaagggatccaattgcgcatgtgatcaattccgctgcggtaacggtaaatgtattccagaggcgtggaaatgtaataacatggatgaatgcggtgatagtagtgacgaagaaatttgcgcaaaggaagccaatcctccgacagcagcagcctttcaaccttgcgcttacaaccagtttcaatgcctttcacggttcacgaaggtatatacatgtctcccagaatcacttaagtgcgacggtaacatagactgtctcgatctcggtgacgaaatcgattgtgacggaggatcccatataaacgaatgcttggtaaataatggt

SEQ ID NO: 38 - DNA, 411 bp, synthetic construct
  source 1..411: mol_type = other DNA
atggactacaaggacgacgatgacaagggatccttttgtctcccgtgggaaattccctgtggggggaattgggggtgctacacggaacaacagaggtgtgacgggtattggcattgccctaacggccgagatgaaacgaactgtactatgtgccaaaaagaggagttcccttgttcacgaaatggagtttgttatccccgatctgatcgatgcaattatcaaaatcattgtccgaatgggagcgacgagaaaaactgctttttttgccagcctgggaatttccattgtaagaacaaccgctgtgtttttgaaagctgggtctgcgattctcaagacgattgcggggacggcagcgatgaagaaaactgcccaggaggatcccatataaacgaatgcttggtaaataatggt

SEQ ID NO: 39 - DNA, 471 bp, synthetic construct
  source 1..471: mol_type = other DNA
atggactacaaggacgacgatgacaagggatccgcctgtatcaccttgacaaaccggacaggattcttgtgtcatgatcagcgaagctgtatacctgcctctggggtctgtgatggtgtgagaacctgcacccacggtgaggacgaggacgaaagtttgtgtcgagatgtgccgcaaagcctgccacacttcctcgtagcgcactgtggtgatcctgcctcctggatttattcagatcaaaagtgtgatgggacaaataattgtggggattgcagcgacgaactttctcccgtgacggtgtgccccccatgcggtcccggttggtggcggtgtcctagcacgtttttcaaatactgcgactgcattccacgacatctctgcagggaccatgtccaacattgttcagactggtcagatgagtacgcttgccccggaggatcccatataaacgaatgcttggtaaataatggt

SEQ ID NO: 40 - DNA, 201 bp, synthetic construct
  source 1..201: mol_type = other DNA
atggactacaaggacgacgatgacaagggatccccatgtggagcctattttaggtgtcaaaacggccgctgtatcccatcaagtcttgtctgcgatccatgggggatggacaactgcggagatggcagtgaccaagggtcctggagcccggctgattgccgcggaggatcccatataaacgaatgcttggtaaataatggt

SEQ ID NO: 41 - DNA, 435 bp, synthetic construct
  source 1..435: mol_type = other DNA
atggactacaaggacgacgatgacaagggatccgagtgtaacattccaggcaatttcatgtgctccaacggcagatgtattccgggtgcttggcagtgtgatggtttgcccgattgctttgataaaagtgatgaaaaggagtgtcctaaggcaaagtcaaaatgcggtccgaccttcttcccttgtgccagcggaatccattgtatcataggacggttcaggtgtaacggatttgaggattgtccggacggttcagatgaggaaaactgtacggccaacccgttgctgtgtagtactgctcgctaccactgcaaaaacgggctctgcatagacaagagtttcatttgcgacggacagaataactgtcaggacaacagcgacgaagaatcttgtgaaggaggatcccatataaacgaatgcttggtaaataatggt

SEQ ID NO: 42 - DNA, 381 bp, synthetic construct
  source 1..381: mol_type = other DNA
atggactacaaggacgacgatgacaagggatccaagtttacttgcacgtccggcaagtgtttgtaccttggtagcctggtttgcaatcaacaaaatgattgcggggacaattcagatgaagaaaactgtcttggttctaaattcacttgtaccagtgggaagtgtttgtacttgggcagtcttgtttgtaaccagcaaaatgattgcggcgataattctgacgaagagaactgccttgggagcaaatttacctgtactagcggaaagtgtctttacttgggtagcctcgtctgtaaccaacaaaacgactgtggtgataactctgatgaagagaactgtttgggaggatcccatataaacgaatgcttggtaaataatggt

SEQ ID NO: 43 - DNA, 1047 bp, synthetic construct
  source 1..1047: mol_type = other DNA
atggactacaaggacgacgatgacaagggatccgccaaatgcgagccaagtcagttccagtgcaccaatggtcgctgtattactttgctgtggaagtgcgacggggacgaggattgtgttgacggatcagacgagaagaactgtgttaagaaaacgtgcgccgagtcagatttcgtttgcaacaatggccaatgcgttccgtctcggtggaagtgcgatggtgatcccgactgcgaagacggtagtgatgaatctcctgagcaatgccacatgcgaacctgtagaattcacgagatcagttgcggagctcactcaacacagtgcattcccgtctcctggaggtgcgacggtgagaacgattgcgattccggagaagacgaggaaaattgcggtaacattacgtgctccccagatgaattcacctgctcatccgggaggtgcatatcccgcaactttgtgtgcaacggacaggatgattgctccgacggaagtgacgagcttgattgcgccccgcctacttgcggggctcacgagtttcaatgctccacgagcagctgcatccccattagttgggtttgtgatgatgatgcggattgctctgaccagtcagacgaaagcctcgaacaatgcggcagacagcccgtgatacacaccaaatgccctgcatctgagatacagtgtggctccggggagtgtatccacaagaaatggcgctgcgatggggacccagactgtaaggacgggtccgatgaagttaattgtcccagtcgaacatgccggcctgaccaattcgaatgcgaagacggttcttgcatacatggttctagacaatgcaacgggatccgagattgtgtggatgggtccgacgaggtgaactgcaaaaatgtcaatcaatgtctcggccccgggaaatttaaatgtagatctggtgaatgtatcgatatttcaaaagtatgtaatcaagaacaggattgccgagactggtccgatgaacctttgaaggagtgtggaggatcccatataaacgaatgcttggtaaataatggt

SEQ ID NO: 44 - DNA, 75 bp, synthetic construct
  source 1..75: mol_type = other DNA
ggcaggtgctgccacaccgctctaatcgcatcgacatccgagtacacactgctgcttaacaacctggagaatgcc

SEQ ID NO: 45 - DNA, 59 bp, synthetic construct
  source 1..59: mol_type = other DNA
aatcgcatcgacatccggcaggtgctgccacactgctgcttaacaacctggagaatgcc

SEQ ID NO: 46 - DNA, 75 bp, synthetic construct
  source 1..75: mol_type = other DNA
aatcgcatcgacatccggcaggtgctgccacaccgctctgagtacacactgctgcttaacaacctggagaatgcc

SEQ ID NO: 47 - DNA, 53 bp, synthetic construct
  source 1..53: mol_type = other DNA
aatcgcatcgacatccggctctgagtacacactgctgcttaacaacctggaga

SEQ ID NO: 48 - DNA, 74 bp, synthetic construct
  source 1..74: mol_type = other DNA
aatcgcatcgacatccggcaggtgctgccacacgctctgagtacacactgctgcttaacaacctggagaatgcc

(19) United States
(12) Patent Application Publication
(10) Pub. No.: US 2026/0327303 A1
(43) Pub. Date: Oct. 22, 2026

(54) RECEPTORS FOR YELLOW FEVER AND RELATED VIRUSES

(71) Applicant: Washington University, St. Louis, MO (US)

(72) Inventors: Michael Diamond, St. Louis, MO (US); Zhenlu Chong, St. Louis, MO (US); Daved Fremont, St. Louis, MO (US); Sean Hui, St. Louis, MO (US)

(73) Assignee: Washington University, St. Louis, MO (US)

(21) Appl. No.: 19/647,576

(22) Filed: Apr. 14, 2026

Related U.S. Application Data
(60) Provisional application No. 63/788,542, filed on Apr. 14, 2025.

(51) Int. Cl. C07K 16/28; A61K 38/00; A61K 39/00; A61P 31/14; C07K 14/705

(52) U.S. Cl. CPC C07K 16/28; A61P 31/14; C07K 14/705; A61K 38/00; A61K 2039/505; C07K 2319/30

(57) ABSTRACT

Among the various aspects of the present disclosure is the provision of a method of treating a viral infection and related compositions. The present teachings include methods for treating a viral infection in a subject in need by administering a low-density lipoprotein receptor (LDLR) inhibitor, as well as LDLR inhibitor compositions.

### CROSS-REFERENCE TO RELATED APPLICATIONS

This application claims the benefit of priority to U.S. Provisional Application Ser. No. 63/788,542 filed on Apr. 14, 2025, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention (file name “021452-US-NP_Sequence-listing_04.06.26” created on 6 Apr. 2026; 65,175 bytes). The subject matter of the Sequence Listing is incorporated herein by reference in its entirety.

### FIELD OF THE INVENTION

The present disclosure generally relates to the identification of entry receptors for yellow fever virus and related members of its antigenic complex and associated inhibitors.

### BACKGROUND OF THE INVENTION

Orthoflaviviruses are a genus of arthropod-transmitted viruses in the Flaviviridae family. There are more than 70 viruses in this genus including yellow fever (YFV), dengue (DENV), Zika (ZIKV), West Nile (WNV), Japanese encephalitis (JEV), and tick-borne encephalitis (TBEV) viruses. Orthoflaviviruses have an approximately 11 kilobase single-stranded, positive-sense RNA genome that encodes three structural (C, prM, and E) and seven non-structural (NS1, NS2A, NS2B, NS3, NS4A, NS4B, and NS5) proteins. Infections in humans can cause mild to severe disease including multi-organ failure, hemorrhage, meningitis, encephalitis, and congenital malformations.

YFV, the prototype of the Orthoflavivirus genus, is transmitted to humans principally by Aedes aegypti mosquitoes and replicates in cells of the liver, kidney, bloodstream, and less frequently, the brain. Approximately 30% of YFV-infected humans develop severe acute hepatic disease, and these cases have a high (approximately 50%) fatality rate. During the 17th to 19th centuries, YFV caused epidemics that threatened millions of people. However, the introduction of vector control measures and deployment of the live-attenuated YFV-17D vaccine in 1938 resulted in markedly reduced transmission rates and disease. Nonetheless, YFV remains a global health threat, particularly in tropical and subtropical regions of Africa and South America, where it causes tens of thousands of deaths each year in part due to vaccine shortages and sporadic vaccination coverage. To date, there is no specific antiviral treatment for YFV or any other orthoflavivirus. The potential for urban epidemic outbreaks with a direct human-mosquito-human transmission cycle, the geographic expansion of its Aedes aegypti mosquito vector due to climate change, and its high case-fatality rate make yellow fever a persistent threat.

The identification of receptors for viral entry is important for understanding tropism and pathogenesis and developing targeted antiviral therapies. Compared to the distantly related positive-sense alphaviruses, for which several receptors (e.g., MXRA8, LDLRAD3, VLDLR, ApoER2/LRP8, and PCDH10) have been identified through CRISPR-Cas9-based screens, less is known about the molecules that mediate attachment and internalization of orthoflaviviruses. Although some cell surface proteins including T-cell immunoglobulin and mucin domain 1 (TIM-1), dendritic cell-specific intercellular adhesion molecule-3-grabbing non-integrin (DC-SIGN), CD300a, neural cell adhesion molecule (NCAM1) and the receptor tyrosine kinase AXL, are implicated in entry of some orthoflaviviruses (e.g., DENV, ZIKV, JEV, TBEV, and WNV), their loss in vivo in mice or cell culture has limited effects on infection and pathogenesis, suggesting functional redundancy or subordinate roles, and few have attributed functions for YFV.

### SUMMARY OF THE INVENTION

Among the various aspects of the present disclosure is the provision of a method of treating a viral infection and related compositions.

The present teachings include methods for treating a viral infection in a subject in need by administering a therapeutically effective amount of at least one low-density lipoprotein receptor (LDLR) inhibitor. In one aspect, the viral infection can be an orthoflaviviral infection. In another aspect, the orthoflaviviral infection can be caused by a virus selected from yellow fever (YFV), dengue (DENV), Zika (ZIKV), West Nile (WNV), Japanese encephalitis (JEV), tick-borne encephalitis (TBEV) viruses, Banzi (BANV), Uganda S (UGSV), and Wesselsbron (WESSV). In another aspect, the LDLR inhibitor is selected from an LDLR-related protein 1 (LRP1) inhibitor, LRP1B inhibitor, LRP2 inhibitor, LRP3 inhibitor, LRP4 inhibitor, VLRDL inhibitor, and any combination thereof. In another aspect, the LDLR inhibitor is selected from a recombinant Fc fusion decoy protein, a soluble protein, and a monoclonal antibody. In another aspect, the recombinant Fc fusion decoy protein comprises at least a portion of a ligand binding domain (LBD). In another aspect the recombinant Fc fusion decoy protein is selected from a LRP1 recombinant Fc fusion (LRP1-Fc) decoy protein, a LRP4-Fc decoy protein, a VLDLR-Fc decoy protein, a LRP4-LBD-Fc decoy protein, a VLDLR-LBD-Fc decoy protein, and any combination thereof. In another aspect, the at least a portion of the LBD comprises at least two LA domains, wherein the at least two LA domains comprise at least one LA repeat. In another aspect, the recombinant Fc fusion decoy protein comprising at least two LA domains is selected from a LRP1-CL-1-Fc decoy protein, a VLDLR-LA(1-2)-Fc decoy protein, a LRP4-LA(2-3)-Fc decoy protein, a LRP4-LA(6-7)-Fc decoy protein, a LRP4-LA(1-3)-Fc decoy protein, aLRP4-LA(2-4)-Fc decoy protein, a LRP4-LA(2-4)-3×-Fc decoy protein, a LRP4-LA(5-7)-Fc decoy protein, a LRP4-LA(6-8)-Fc decoy protein, a LRP4-LA(2,3,6,7)-Fc decoy protein, and any combination thereof. In another aspect, the LDLR inhibitor is the LRP4-LA(2,3,6,7)-Fc decoy protein, the LRP1-CL-I-Fc decoy protein, and the VLDLR-LA(1-2)-Fc decoy protein. In another aspect, administering the therapeutically effective amount of the LDLR inhibitor comprises administering 50 mg per kg of the LDLR inhibitor.

The present teachings also include a composition for treating a viral infection. The composition comprising a Fc domain, a low-density lipoprotein receptor (LDLR) family member protein, and a pharmaceutically acceptable carrier, wherein the LDLR family member protein comprises at least a portion of a ligand binding domain (LBD), wherein the LBD comprises at least two LA domains, and the LA domain comprises one or more LA repeats. In some aspects, the viral infection is an orthoflavivirus infection caused by a virus selected from yellow fever (YFV), dengue (DENV), Zika (ZIKV), West Nile (WNV), Japanese encephalitis (JEV), tick-borne encephalitis (TBEV) viruses, Banzi (BANV), Uganda S (UGSV), and Wesselsbron (WESSV). In another aspect, the LDLR family member protein is selected from an LDLR-related protein 1 (LRP1), LRP1B, LRP2, LRP3, LRP4, VLRDL, and any combination thereof. In another aspect, the composition is selected from a LRP1 recombinant Fc fusion (LRP1-Fc) decoy protein, a LRP4-Fc decoy protein, a VLDLR-Fc decoy protein, a LRP4-LBD-Fc decoy protein, a VLDLR-LBD-Fc decoy protein, a LRP1-CL-1-Fc decoy protein, a VLDLR-LA(1-2)-Fc decoy protein, a LRP4-LA(2-3)-Fc decoy protein, a LRP4-LA(6-7)-Fc decoy protein, a LRP4-LA(1-3)-Fc decoy protein, aLRP4-LA(2-4)-Fc decoy protein, a LRP4-LA(2-4)-3×-Fc decoy protein, a LRP4-LA(5-7)-Fc decoy protein, a LRP4-LA(6-8)-Fc decoy protein, a LRP4-LA(2,3,6,7)-Fc decoy protein, and any combination thereof. In another aspect, the composition is the LRP4-LA(2,3,6,7)-Fc decoy protein, the LRP1-CL-I-Fc decoy protein, and the VLDLR-LA(1-2)-Fc decoy protein.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a plot of MAGeCK analysis for a CRISPR screen with YFV-17D showing enriched genes based on top robust rank aggregation (RRA) scores or False Discovery Rate adjusted P values (FDR).

FIG. 1B is a set of graphs of relative infection of HAP1 ΔHS cells. HAP1 ΔHS (control), ΔLRP4 and LRP4-complemented ΔLRP4 HAP1 ΔHS (ΔLRP4+LRP4) cells were inoculated with YFV-17D (MOI of 1; left) for 16 h, or YFV-17D RVPs (right) expressing GFP (1×10⁴ |U) for 24 h, and infection was measured by flow cytometry. Data are mean±standard deviation; one-way ANOVA with Dunnett's post-test; ****p<0.001.

FIG. 1C is a graph showing infection of K562 cells, control K562 cells and those expressing N-terminal Flag-tagged LRP4 (Flag-LRP4), inoculated with YFV-17D (MOI of 1) for 24 h, and infection was measured by flow cytometry. Data are mean±standard deviation; unpaired t-test; ****p<0.001.

FIG. 1D is a plot of a focus-forming assay (FFA) of control K562 cells and those expressing Flag-LRP4 inoculated with YFV-17D (MOI of 0.01), and viral yield in the supernatant was measured over time. Data are ±standard error of the mean (s.e.m.) from 3 independent experiments; one-way ANOVA; ****p<0.001.

FIG. 1E is a graph of control K562 cells and those expressing Flag-LRP4 inoculated with indicated YFV strains for 24 h (Dakar, INS, INH, M185D, Kouma, MOI of 5) or 48 h (Asibi, MOI of 1), and infection was measured by flow cytometry. Data are ±s.e.m.; one-way ANOVA; ****p<0.001.

FIG. 1F is a phylogenetic tree generated from E protein sequences of indicated orthoflaviviruses with members of the YFV complex highlighted in brown. Data are mean±standard deviation; one-way ANOVA; ****p<0.001.

FIG. 1G is a graph of control K562 cells and those expressing Flag-LRP4 inoculated with indicated viruses for 24 h (Banzi, MOI of 1; Entebbe bat, MOI of 5) or 48 h (Uganda S, MOI of 1; Wesselsbron, MOI of 1), and infection was measured by flow cytometry. Data are ±s.e.m.; one-way ANOVA; ****p<0.001.

FIG. 1H is a graph of control K562 cells and those expressing Flag-LRP4 inoculated with indicated viruses for 24 h (DENV1, DENV2, DENV3, DENV4, MOI of 10; LGTV, WNV-NY99, WNV-Kunjin, ZIKV, MOI of 3), and infection was measured by flow cytometry. Data are mean±standard deviation; two-way ANOVA with Šídak's multiple comparisons test; ns, not significant.

FIG. 1I is a graph of control K562 cells and those expressing Flag-LRP4 inoculated with indicated viruses for 8 h (RRV, SINV-WEEV, SINV-VEEV, and SINV-SFV, MOI of 3), and infection was measured by flow cytometry. Data are mean±standard deviation; two-way ANOVA with Šídák's multiple comparisons test; ns.

FIG. 2A is a qRT-PCR graph of YFV-17D which was incubated with control K562 cells or those expressing Flag-LRP4 at 4 °C. Bound virions (viral RNA, vRNA) and GAPDH RNA levels were quantified. Data are mean±s.d. of 3 independent experiments; unpaired t-test; ***p<0.001.

FIG. 2B is a qRT-PCR graph of YFV-17D which was incubated with control K562 cells or those expressing Flag-LRP4 at 4 °C. After the removal of unbound virus, the temperature was increased to 37 °C to allow for internalization. Intracellular RNA (YFV-17D and GAPDH) levels were measured. Data are mean±s.d. of 3 independent experiments; unpaired t-test; ****p<0.0001.

FIG. 2C is a schematic of LRP4 ectodomain and deletion constructs. LBD LA repeats are numbered.

FIG. 2D is a graph of percent infection of K562 cells expressing the constructs shown in FIG. 2C with YFV-17D virus and measured by flow cytometry at 24 h. Data are mean±s.d. of 3 independent experiments; one-way ANOVA; ****p<0.0001.

FIG. 2E is a graph of percent infection of K562 cells expressing the constructs shown in FIG. 2C with RVPs and measured by flow cytometry at 24 h. Data are mean±s.d. of 3 independent experiments; one-way ANOVA; ****p<0.0001.

FIG. 2F is a graph of percent infection of control K562 cells or those expressing indicated LRP4 orthologs with YFV-17D RVPs and measured by flow cytometry at 24 h. Data are mean±s.d. of 3 independent experiments; one-way ANOVA; **p<0.01, ***p<0.001, ****p<0.0001.

FIG. 2G is a graph and associated schematic of the binding of LRP4-LBD-Fc or LDLRAD3-LA1-Fc proteins to YFV-17D. Plates were coated with anti-E mAb and binding was measured by ELISA. Data are mean±s.d. of 3 independent experiments.

FIG. 2H is a graph and associated schematic of the binding of LRP4-LBD-Fc or LDLRAD3-LA1-Fc proteins to YFV-17D. Plates were coated with Fc fusion proteins and binding was measured by ELISA. Data are mean±s.d. of 3 independent experiments.

FIG. 2I is a plot and associated schematic of the binding of YFV-M185D/BinJ virions to the indicated Fc fusion proteins or mAbs by biolayer interferometry (BLI). Biosensors were coated with YFV-M185D/BinJ after absorption with Fab fragments of an anti-YFV mAb and then incubated with Fc-fusion proteins or mAbs in solution. Data are representative results from 2 independent experiments.

FIG. 2J is a plot of the binding of YFV-M185D/BinJ virions to the indicated Fc fusion proteins or mAbs by BLI. Biosensors were coated with indicated Fc-fusion proteins or mAbs and then incubated with YFV-M185D/BinJ. Data are representative results from 2 independent experiments.

FIG. 2K is a plot of the binding of YFV-Asibi/BinJ virions to the indicated Fc fusion proteins or mAbs by BLI. Biosensors were coated with indicated Fc-fusion proteins or mAbs and then incubated with YFV-Asibi/BinJ. Data are representative results from 2 independent experiments.

FIG. 2L is a plot and associated schematic of the binding of YFV-ES504/BinJ virions to the indicated Fc fusion proteins or mAbs by BLI. Biosensors were coated with indicated Fc-fusion proteins or mAbs and then incubated with YFV-ES504/BinJ. Data are representative results from 2 independent experiments.

FIG. 2M is a plot and associated schematic of the binding of YFV-Asibi E-DIII (trimeric) protein to LRP4-LBD-Fc fusion protein by BLI. Biosensors were coated with LRP4-LBD-Fc fusion protein, and YFV-Asibi E-DIII trimeric protein was added in solution. Data are representative results from 2 independent experiments.

FIG. 2N is a plot of SPR sensorgrams and kinetic fits of monomeric YFV-Asibi E-DIII protein binding to LRP4-LBD-Fc. LRP4-LBD-Fc and LDLRAD3-LA1-Fc (negative control) were immobilized on the sensor surface, and YFV-Asibi E-DIII protein was injected (range of 1.3 μM to 320 μM; 1:3 dilution series). The response from the LDLRAD3-LA1-Fc channel was subtracted from the LRP4-LBD-Fc response. A custom Python script was used to fit curves and kinetic parameters for a 2-site binding model. K_{D} was calculated from ka and ka from each interaction (n=2, representative sensorgram). Data are representative results from 2 independent experiments.

FIG. 2O is a plot of equilibrium analyses of monomeric YFV-Asibi E-DIII protein binding to LRP4-LBD-Fc experiment. Response units from each channel were normalized to the maximum binding response on that channel. Hill-Langmuir and Scatchard models capturing 2 independent sites were implemented in a custom Python script to determine the steady state parameters (n=2). Data are representative results from 2 independent experiments.

FIG. 2P is a plot and associated schematic of the binding of YFV-Asibi E-DIII mutant proteins to LRP4-LBD-Fc fusion protein by BLI (top) abs a ribbon diagram of a YFV-Asibi E-DIII mutant protein. Biosensors were coated with LRP4-LBD-Fc fusion protein and YFV-Asibi E-DIII mutant proteins were added in solution. Mutations in bold indicate loss of binding (red line). Data are representative results from 2 independent experiments.

FIG. 2Q is a graph (left) of percent infection of K562 cells expressing N-terminal Flag-tagged LRP4 (Flag-LRP4) inoculated with YFV-17D RVPs containing indicated mutations in E for 24 h, and a ribbon diagram of the YFV-17D

RVPs containing the mutations in E (right). Infection was measured by flow cytometry. Data are mean±s.d. from 3 independent experiments; one-way ANOVA; ****p<0.0001.

FIG. 3A is a plot of percent infection of HAP1 cells showing the inhibition of YFV-17D infection with indicated concentrations of LRP4-LBD-Fc or LDLRAD3-LA1-Fc fusion proteins. Data shown are mean±s.d. of 3 independent experiments.

FIG. 3B is a plot of percent infection of HepG2 cells showing the inhibition of YFV-17D infection with indicated concentrations of LRP4-LBD-Fc or LDLRAD3-LA1-Fc fusion proteins. Data shown are mean±s.d. of 3 independent experiments.

FIG. 3C is a plot of percent infection of HAP1 cells showing the inhibition of YFV-Asibi infection with indicated concentrations of LRP4-LBD-Fc or LDLRAD3-LA1-Fc fusion proteins. Data shown are mean±s.d. of 3 independent experiments.

FIG. 3D is a plot of percent infection of HAP1 cells showing the inhibition of YFV-M185D infection with indicated concentrations of LRP4-LBD-Fc or LDLRAD3-LA1-Fc fusion proteins. Data shown are mean±s.d. of 3 independent experiments.

FIG. 3E is a plot showing the inhibition of YFV-17D infection in HAP1 cells with indicated concentrations of LRP4-Fc fusion proteins containing three LA domain repeats. Data shown are mean values of 3 independent experiments.

FIG. 3F is a set of qRT-PCR graphs showing PFU in the indicated tissue samples. 500 μg of LRP4-LA(2367)-Fc or LDLRAD3-LA1-Fc protein were incubated with 10⁵ PFU of YFV-17D at 37 °C for 1 h and then inoculated into anti-IFNAR1 antibody treated C57BL/6J mice via intraperitoneal injection. Mice were also given 500 μg of LRP4-LA(2367)-Fc or LDLRAD3-LA1-Fc protein at 1- and 2-days post infection (n=12 and 10, respectively). At three days post infection, viral RNA levels in indicated tissues were measured by qRT-PCR. Data shown are median values of 2 independent experiments; Mann-Whitney test; *P<0.05, **P <0.01, ***P<0.001, ****P<0.0001.

FIG. 3G is a set of qRT-PCR graphs showing PFU in the indicated tissue sample. Littermate wild-type and Lrp4^{hypo/hypo} mice were treated with 2 mg of anti-IFNAR1 antibody. One day later, animals were inoculated with 10⁵ PFU of YFV-M185D via intraperitoneal injection (n=17 and 16, respectively). At five days post-infection, tissues were collected, and viral RNA levels in indicated tissues were measured by qRT-PCR. Data shown are median values of 2 independent experiments; Mann-Whitney test; *P<0.05, n.s.

FIG. 4A is a graph showing percent infection measured by flow cytometry. Control K562 cells and those expressing N-terminal Flag-tagged LDLR family members were inoculated with YFV-17D RVP for 24 h. Data are mean±s.d. of 3 independent experiments; one-way ANOVA with Dunnett's post-test; ****p<0.0001.

FIG. 4B is a graph showing percent infection of K562 cells. Control K562 cells and those expressing LRP1-CL-I, LRP1B-CL-III and VLDLR were inoculated with YFV-Asibi for 48 h (MOI of 1), and infection was measured by flow cytometry. Data are mean±s.d. of 3 independent experiments; one-way ANOVA with Dunnett's post-test; ****p<0.0001.

FIG. 4C is a graph showing percent infection of K562 cells. Control K562 cells and those expressing LRP1-CL-I, LRP1B-CL-III and VLDLR were inoculated with YFV-M185D for 24 h (MOI of 5), and infection was measured by flow cytometry. Data are mean±s.d. of 3 independent experiments; one-way ANOVA with Dunnett's post-test; **p<0.01, ****p<0.0001.

FIG. 4D is a graph showing percent infection of K562 cells. Control K562 cells and those expressing LRP1-CL-I, LRP1B-CL-III and VLDLR were inoculated with Banzi, for 24 h (MOI of 1), and infection was measured by flow cytometry. Data are mean±s.d. of 3 independent experiments; one-way ANOVA with Dunnett's post-test; *p<0.05, ****p<0.0001.

FIG. 4E is a graph showing percent infection of K562 cells. Control K562 cells and those expressing LRP1-CL-I, LRP1B-CL-III and VLDLR were inoculated with Uganda S for 48 h (MOI of 1), and infection was measured by flow cytometry. Data are mean±s.d. of 3 independent experiments; one-way ANOVA with Dunnett's post-test; ****p<0.0001.

FIG. 4F is a graph of percent infection of control HepG2 cells which were inoculated with YFV-17D (MOI of 2) in the present of indicated proteins (10 μg/mL) for 16 h, and infection was measured by flow cytometry. Data are mean±s.d. of 3 independent experiments; one-way ANOVA with Šidák post-test; ****p<0.0001.

FIG. 4G is a graph of percent infection of WT and ΔLRP1 HepG2 cells which were inoculated with YFV-17D (MOI of 2) in the present of indicated proteins (10 μg/mL) for 16 h, and infection was measured by flow cytometry. Data are mean±s.d. of 3 independent experiments; one-way ANOVA with Šidák post-test; *p<0.05, ****p<0.0001.

FIG. 4H is a graph of percent infection of WT and ΔLRP4 HepG2 cells which were inoculated with YFV-17D (MOI of 2) in the present of indicated proteins (10 μg/mL) for 16 h, and infection was measured by flow cytometry. Data are mean±s.d. of 3 independent experiments; one-way ANOVA with Šidák post-test; ***p<0.001, ****p<0.0001.

FIG. 4I is a graph of percent infection of WT and ΔVLDLR HepG2 cells which were inoculated with YFV-17D (MOI of 2) in the present of indicated proteins (10 μg/mL) for 16 h, and infection was measured by flow cytometry.

FIG. 4J is a plot showing the binding of YFV virions to LRP1-CL-I-Fc fusion proteins by BLI. Biosensors were coated with Fc fusion proteins and then incubated with YFV-M185D/BinJ virions in solution. Data are results from 2 independent experiments.

FIG. 4K is a plot and associated schematic showing the binding of YFV virions to VLDLR-LA(1-2)-Fc fusion proteins by BLI. Biosensors were coated with Fc fusion proteins and then incubated with YFV-M185D/BinJ virions in solution. Data are results from 2 independent experiments.

FIG. 4L is a plot of percent infection showing the inhibition of YFV-17D infection with indicated concentrations of LRP1-CL-I-Fc or VLDLR-LA(1-2)-Fc fusion proteins in HepG2 cells. Data are mean±s.d. of 3 independent experiments.

FIG. 4M is a set of qPT-PCR graphs showing FFU in the indicated tissues. 500 μg of LRP1-CL-I-Fc or LDLRAD3-LA1-Fc protein were incubated with 10⁵ PFU of YFV-17D at 37 °C for 1 h and then inoculated into anti-IFNAR1 antibody treated C57BL/6J mice via intraperitoneal injection. Mice were also given 500 μg of LRP1-CL-I-Fc or LDLRAD3-LA1-Fc protein at 1- and 2-days post infection (n=5 in each group). At three days post-infection, viral RNA levels in indicated tissues were measured by qRT-PCR. Data are results from 1 independent experiment; Mann-Whitney test; **p<0.01.

FIG. 4N is a survival plot of LRP1-CL-I-Fc, LRP4-LA(2,3,6,7)-Fc, VLDLR-LA(1-2)-Fc or LDLRAD3-LA1-Fc (200 μg) was pre-incubated with 102 PFU of YFV-17D and inoculated into anti-IFNAR1-treated C57BL/6J mice through intracranial injection. n=25 (LDLRAD3-LA1-Fc) and n=10 (the other 3 groups).

FIG. 4O is a schematic diagram of the treatment protocol of AG129 mice in FIG. 4P and FIG. 4Q.

FIG. 4P is a survival plot of AG129 mice. 500 μg of LRP1-CL-I-Fc or LDLRAD3-LA1-Fc was pre-incubated with 105 PFU of YFV-17D and inoculated into AG129 mice through intraperitoneal injection. n=8 and 10.

FIG. 4Q is a survival plot of AG129 mice. 50 mg per kg of LRP1-CL-I-Fc or LDLRAD3-LA1-Fc was administered to AG129 mice 6 h before intraperitoneal inoculation with 105 PFU of YFV-17D. n=12 and 11.

FIG. 5A is a graph of percentage of initial body weight. LRP1-CL-I-Fc or LDLRAD3-LA1-Fc protein (50 mg per kg) was administered to hFRG mice (engrafted with human hepatocytes) by intraperitoneal injection at 6 h before challenge with 2×10⁵ focus-forming units (FFU) of YFV-Dakar. hFRG mice were given a second 50 mg per kg dose of LRP1-CL-I-Fc or LDLRAD3-LA1-Fc protein at 24 h.p.i. n=6.

FIG. 5B is a graph of viral RNA levels in the liver at 5 d.p.i from the experimental setup in FIG. 5A.

FIG. 5C is a graph of viral RNA levels in serum at 5 d.p.i from the experimental setup in FIG. 5A.

FIG. 5D of is a graph of prothrombin time in the blood at 5 d.p.i from the experimental setup in FIG. 5A.

FIG. 5E of is a graph of alanine aminotransferase levels in the serum at 5 d.p.i from the experimental setup in FIG. 5A.

FIG. 5F is a set of histology images of liver. Left, mock-infection. Typical liver histology observed in an hFRG mouse engrafted with human hepatocytes. The dashed lines demarcate the boundaries between human (paler) and mouse (more eosinophilic) hepatocytes. Middle, extensive liver histopathology seen in YFV-infected hFRG mice treated with LDLRAD3-LA1-Fc. A patch of human hepatocyte dropout (absence of nuclear and cytoplasmic features) is delineated by the dashed yellow line. Abundant Councilman-Rocha-Lima-like bodies are highlighted by the yellow arrows. Right, the hepatic architecture is preserved in the YFV-infected mice treated with LRP1-CL-I-Fc protein. ×200 magnification. BD, bile duct; CV, central vein; HA, hepatic artery; PV, portal vein. Scale bar, 200 μm.

FIG. 5G is a graph of control and ΔLRP1 PHHs were inoculated with YFV-M185D (MOI, 10) in the presence of ruxolitinib. At 48 h, infection was measured using flow cytometry (anti-E mAbs).

FIG. 6A is a schematic diagram showing a CRISPR-Cas9 screening methodology. HAP1 ΔHS cells were transduced with the cell surface library containing 4,630 sgRNAs, selected with puromycin, and inoculated with YFV-17D at an MOI of 10. Surviving cells were expanded, and 3 weeks later, genomic DNA from cells was harvested and sequenced for sgRNA abundance.

FIG. 6B is an image of HAP1 ΔHS sequences. Next-generation sequencing confirmation of LRP4 gene editing in HAP1 ΔHS cells.

FIG. 6C is a set of flow cytometry plots of HAP1 ΔHS (Control), HAP1 ΔHS ΔLRP4 (ΔLRP4) and LRP4-complemented HAP1 ΔHS ΔLRP4 (ΔLRP4+LRP4) cells were analyzed for surface expression of LRP4 by flow cytometry using an anti-Flag mAb.

FIG. 6D is an image of HAP1 ΔHS sequences. Next-generation sequencing confirmation of LRP4 gene editing in 293T cells.

FIG. 6E is a graph showing percent infection of 293T cells. 293T (control), ΔLRP4 and LRP4-complemented ΔLRP4 293T (ΔLRP4+LRP4) cells were inoculated with YFV-17D (MOI of 20) for 16 h, and infection was measured by flow cytometry. Data are mean±s.d. of 3 independent experiments with one-way ANOVA with Dunnett's post-test; ****P<0.0001.

FIG. 6F is a set of flow cytometry plots of control K562 cells and those expressing Flag-LRP4 were analyzed for surface expression of LRP4 by flow cytometry using an anti-Flag mAb.

FIG. 6G is a set of flow cytometry plots of control and Flag-LRP4 expressing K562 cells were analyzed for surface expression of LRP4 by flow cytometry (anti-LRP4 mAb).

FIG. 6H is a set of flow cytometry plots of control and Flag-LRP4 expressing K562 cells were analyzed for surface expression of LRP4 by flow cytometry (anti-Flag mAb)

FIG. 6I is a graph of percent infection of K562 cells. Entebbe bat virus (MOI of 5) was incubated with mE60 antibody or isotype control antibody at 200 ng/ml for 1 h at 37 °C and then infected K562 cells. Infection levels were measured by flow cytometry at 24 h post-infection. Data are mean±s.d. of 3 independent experiments; unpaired t-test; ***P<0.001, ****P<0.0001.

FIG. 7A is a set of flow cytometry plots of K562 cells ectopically expressing indicated LRP4 variants were measured by flow cytometry using an anti-Flag mAb. Data are representative of 2 independent experiments.

FIG. 7B is a set of flow cytometry plots of K562 cells ectopically expressing LRP4 orthologs were measured by flow cytometry using an anti-Flag mAb. Data are representative of 2 independent experiments.

FIG. 7B is a graph of K562 cells ectopically expressing LRP4 orthologs measured by flow cytometry (anti-Flag mAb).

FIG. 7C is a graph summarizing mean fluorescence intensity (MFI) from flow cytometry results of FIG. 7B.

FIG. 8A is an image of a DS-PAGE gel with Coomassie staining of purified human LRP4-LBD-Fc and LDLRAD3-LA1-Fc proteins.

FIG. 8B is an image of an SDS-PAGE gel with Coomassie staining of YFV-M185D/BinJ (fraction in lane 2 was used for BLI experiments).

FIG. 8C is an image of an SDS-PAGE gel with Coomassie staining of YFV-ES504/BinJ (fraction in lane 3 was used for BLI experiments) and YFV-Asibi/BinJ (fraction in lane 7 was used for BLI experiments).

FIG. 8D is a set of micrograph images of YFV-Asibi/BinJ (top) and YFV-M185D/BinJ (bottom) chimeric virions showing mature and immature particles of approximately 50 nm in diameter. Scale bar, 100 nM.

FIG. 8E is a plot showing the inhibition of YFV-17D infection with indicated concentrations of LRP4-LBD-Fc fusion protein in 293T cells (n=3).

FIG. 8F is a plot showing the inhibition of YFV-17D infection with indicated concentrations of LRP4-LBD-Fc fusion protein in Vero cells (n=3).

FIG. 8G is a plot showing the inhibition of YFV-17D infection with indicated concentrations of LRP4-Fc fusion proteins containing only one LA domain repeats in HAP1 cells (n=3).

FIG. 8H is a plot showing the inhibition of YFV-17D infection in HAP1 cells with indicated concentrations of LRP4-Fc fusion proteins containing two LA domain repeats (n=3).

FIG. 8I is a graph showing percent infection of control K562 cells or those expressing indicated LRP4 variants with YFV-17D RVPs at 24 h was measured by flow cytometry (n=3).

FIG. 8J is a graph showing percent infection of control K562 cells or those expressing indicated LRP4 variants with YFV-17D RVPs at 24 h was measured by flow cytometry (n=3).

FIG. 8K is a graph showing percent infection of control K562 cells or those expressing indicated LRP4 variants with YFV-17D RVPs at 24 h was measured by flow cytometry (n=3).

FIG. 8L is a set of flow cytometry plots of K562 cells ectopically expressing indicated LRP4 variants in FIG. 7H, FIG. 7I, and FIG. 7J and were measured by flow cytometry using an anti-Flag mAb (n=3).

FIG. 9A is a graph and associated schematic showing binding of YFV-Asibi E-DIII mutant proteins to anti-YFV Adi-49147 mAb by BLI. Biosensors were coated with Adi-49147 mAb following incubation with YFV-Asibi E-DIII mutant proteins in solution (n=2).

FIG. 9B is a graph of infection of YFV-17D RVPs with indicated mutations in E-DIll mutant was measured by flow cytometry on Raji-DC-SIGNR cells (GFP expression).

FIG. 10A is a graph of human LRP4 RNA levels shown in indicated tissues. Data were obtained from the Human Protein Atlas database.

FIG. 10B is a graph of human LRP4 protein expression levels.

FIG. 10C is a graph of mouse LRP4 RNA expression levels shown in indicated tissues. Data were obtained from the BioGPS database

FIG. 11A is a set of qRT-PCR graphs of FFU in the indicated tissue. AG129 mice were given 500 μg of LRP4-LBD-Fc (n=7), LRP4-LA(6-8)-Fc (n=7), or LDLRAD3-LA1-Fc (n=9) protein by i.p. at 4 h before infection. Mice were then inoculated with 10⁵ PFU of YFV-17D by i.p route. At 5 dpi, viral RNA levels in indicated tissues were measured by qRT-PCR. Data are pooled from 2 independent experiments; one-way ANOVA with Dunnett's post-test; ***p<0.001.

FIG. 11B is a table of serum levels at day+1 after injection for the indicated LRP4-Fc decoy neutralization activity in vitro.

FIG. 11C is a plot (top) and associated schematic (bottom) showing binding of YFV-M185D/BinJ to indicated LRP4 single LA repeat Fc fusion proteins by BLI. Biosensors were coated with indicated Fc-fusion proteins following incubation with YFV-M185D/BinJ.

FIG. 11D is a plot showing the inhibition of YFV-17D infection with indicated concentrations of LRP4-LA(234)-3×-Fc fusion protein in HAP1 cells.

FIG. 11E is a set of qRT-PCR graphs of FFU in the indicated tissue. 500 μg of LRP4-LA(2-4)-Fc or LDLRAD3-LA1-Fc protein were incubated with 10⁵ PFU of YFV-17D at 37 °C for 1 h and then inoculated into AG129 mice via intraperitoneal injection. Mice were also given 500 μg of LRP4-LA(2-4)-Fc or LDLRAD3-LA1-Fc protein at 1- and 2-days after infection (n=12 and 8, respectively). At three days post-infection, viral RNA levels in indicated tissues were measured by qRT-PCR. Data are from 2 independent experiments; Mann-Whitney test; ***P<0.001, ****P<0.0001.

FIG. 11F is a set of qPR-PCR graphs of FFU in the indicated tissues. Wild-type (n=7) and Lrp4^{hypo/hypo} (n=11) mice were inoculated in the footpad with 10² PFU of VEEV-ZPC738. At 4 dpi, tissues were collected, and viral RNA levels tissues were measured by qRT-PCR. Data are from 2 independent experiments.

FIG. 12A contains schematic diagrams of LDLR family members. The indicated LBD domains were expressed ectopically using lentiviruses.

FIG. 12B is a set of flow cytometry plots of K562 cells ectopically expressing indicated LDLR family members measured using an anti-Flag mAb.

FIG. 13A is a graph of human LRP1 RNA expression levels in the indicated tissues. Data were obtained from Human Protein Atlas database.

FIG. 13B is a graph of mouse LrP1 RNA expression levels in the indicated tissues. Data were obtained from the BioGPS database.

FIG. 13C is a graph of human VLDLR RNA expression levels in the indicated tissues. Data were obtained from Human Protein Atlas database.

FIG. 13D is a graph of mouse VIdlr RNA expression levels in the indicated tissues. Data were obtained from the BioGPS database.

FIG. 14A is a table of LRP1, LRP4 and VLDLR knockout (KO) efficiency (as indicated by sequences of wild-type allele) in HepG2 cells as analyzed by next-generation deep sequencing.

FIG. 14B is a schematic diagram of LRP1-CL-I-Fc, VLDLR-LBD-Fc and VLDLR-LA(1-2)-Fc proteins.

FIG. 14C is an image of an SDS-PAGE gel with Coomassie staining of purified human LRP1-CL-I-Fc and VLDLR-Fc proteins.

FIG. 14D is a plot (left) and associated schematic (right) showing the binding of YFV virions to VLDLR-LBD-Fc fusion protein by BLI. Biosensors were coated with VLDLR-LBD-Fc fusion protein and incubated with YFV-M185D/BinJ virions in solution.

FIG. 14E is a plot of the inhibition of YFV-17D infection with indicated concentrations of VLDLR-LBD-Fc fusion protein in HepG2 cells.

FIG. 14F is a graph of percent infection of control K562 cells or those expressing indicated VLDLR variants with YFV-17D RVPs was measured by flow cytometry at 24 h.

FIG. 14G is a set of flow cytometry plots showing expression of indicated VLDLR tandem LA domain repeats in FIG. 13E using an anti-Flag mAb.

FIG. 14H is a set of qRT-PCR graphs of FFU in the indicated tissues. 500 μg of LRP1-CL-I-Fc or LDLRAD3-LA1-Fc protein was incubated with 105 PFU of YFV-17D at 37 °C for 1 h and then inoculated into anti-IFNAR1 antibody treated C57BL/6J mice via intraperitoneal injection. Mice were also given 500 μg doses of LRP1-CL-I-Fc or LDLRAD3-LA1-Fc protein at 1 and 2 dpi (n=10 and 7, respectively). At 3 d.p.i., viral RNA levels in indicated tissues were measured.

FIG. 14I is a set of qRT-PCR graphs of FFU in the indicated tissues. 500 μg of LDLRAD3-LA1-Fc or VLDLR-LA(1-2)-Fc proteins were incubated with 105 PFU of YFV-M185D at 37 °C for 1 h and then inoculated into anti-IFNAR1 antibody treated C57BL/6J mice via intraperitoneal injection. Mice were also given 500 μg of the same Fc proteins at 1 and 2 dpi (n=10 and 8, respectively). At 3 d.p.i., viral RNA levels in indicated tissues were measured.

FIG. 14J is a set of qRT-PCR graphs of FFU in the indicated tissues. 500 μg of LDLRAD3-LA1-Fc or 1:1:1 mixture (167 μg of each) of LRP1-CL-I-Fc, LRP4-LA (2367)-Fc and VLDLR-LA(1-2)-Fc proteins were incubated with 105 PFU of YFV-M185D at 37 °C for 1 h and then inoculated into anti-IFNAR1 antibody treated C57BL/6J mice via intraperitoneal injection. Animals also were given 500 μg of the same Fc proteins at 1 and 2 dpi (n=10 and 9, respectively). At 3 d.p.i., viral RNA levels in indicated tissues were measured.

FIG. 15A is a table of serum levels of VLDLR-LA(1-2)-Fc and LRP1-CL-I-Fc proteins measured at 1 day post i.p. injection by ELISA.

FIG. 15B is a plot of individual hFRG mouse body weight changes in LDLRAD3-LA1-Fc or LRP1-CL-I-Fc treated groups after YFV infection.

FIG. 15C is a table of LRP1 KO efficiency (as indicated by sequences of wild-type allele) in PHH was analyzed by next generation deep sequencing. Data are from 2 experiments.

FIG. 15D is a set of flow cytometry contour plots showing YFV infection in control and LRP1 KO PHH.

### DETAILED DESCRIPTION OF THE INVENTION

The present disclosure is based, at least in part, on the identification of several members of the LDLR family as entry receptors for yellow fever virus and related members of its antigenic complex by performing a CRISPR genetic screen. This information was used to generate new soluble receptor decoy Fc-fusion protein inhibitors that protect against infection in animals. The identification of this family of proteins as receptors may be important for the larger Flavivirus genus (including dengue, West Nile, and Zika). Characterizing was done on the structural basis of this interaction, which lead to the development of small molecule inhibitors.

As shown herein, it is shown that multiple LDLR family members act as entry receptors for yellow fever virus, and decoy receptors inhibit infection in mice.

The LDLR family members include, but are not limited to, LRP1, LRP1B, LRP2, LRP3, LRP4, and VLDLR.

A LDLR inhibitor can inhibit at least one of an LDLR family member from binding to a ligand such as a virus. An LDLR inhibitor can be a recombinant Fc protein, a soluble protein, and a monoclonal antibody.

In some cases, the recombinant Fc protein can include a LRP1 recombinant Fc fusion (LRP1-Fc) decoy protein, a LRP4-Fc decoy protein, a VLDLR-Fc decoy protein, a LRP4-LBD-Fc decoy protein, a VLDLR-LBD-Fc decoy protein. In some aspect, a soluble protein can bind a LDLR family member. In some cases, the LDLR inhibitor may be a soluble receptor-associated protein (RAP).

In some aspect, the recombinant Fc protein comprises a portion of the ligand binding domain. In some aspect, the LBD comprises at least two or more LA domains. In some aspects, the LA domains may have multiple repeats.

For example, the LDLR inhibitor may be an LRP4-Fc protein, an LRP4-LBD-Fc protein, an LRP4-LA-Fc protein, an LRP4-LA(2-4)-Fc protein, an LRP4-LA(2-4)-3×-Fc protein.

The LDLR inhibitor may be a combination of recombinant Fc proteins. The LDLR inhibitor may be a LRP4-LA(2,3,6,7)-Fc decoy protein, a LRP1-CL-I-Fc decoy protein, and a VLDLR-LA(1-2)-Fc decoy protein combination.

### Low-Density Lipoprotein Receptor (Ldlr) Modulation Agents

As described herein, LDLR expression has been implicated in various diseases, disorders, and conditions. As such, modulation of LDLR (e.g., modulation of LRP4 to inhibit viral infection) can be used for treatment of such conditions. An LDLR modulation agent can modulate LDLR response or induce or inhibit LDLR. LDLR modulation can comprise modulating the expression of LDLR on cells, modulating the quantity of cells that express LDLR, or modulating the quality of the LDLR-expressing cells.

LDLR modulation agents can be any composition or method that can modulate LDLR expression on cells (e.g., an LRP4 inhibitor). For example, an LDLR modulation agent can be an activator, an inhibitor, an agonist, or an antagonist. As another example, the LDLR modulation can be the result of gene editing.

An LDLR modulation agent can be an anti-LDLR antibody (e.g., a monoclonal antibody to LRP4).

An LDLR modulating agent can be an agent that induces or inhibits progenitor cell differentiation into LDLR expressing cells. For example, a decoy protein can be used to block LRP4.

LDLR Signal Reduction, Elimination, or Inhibition by Small Molecule Inhibitors, shRNA, siRNA, or ASOs

As described herein, an LDLR modulation agent can be used for use in viral therapy, including but not limited to orthoflaviviral infection. An LDLR modulation agent can be used to reduce/eliminate or enhance/increase LDLR signals. For example, an LDLR modulation agent can be a small molecule inhibitor of LSR4. As another example, an LDLR modulation agent can be a short hairpin RNA (shRNA). As another example, an LDLR modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### Ldlr Inhibiting Agent

One aspect of the present disclosure provides for targeting of LDLR, its ligand, or its downstream signaling. The present disclosure provides methods of treating or preventing viral infection based on the discovery that multiple LDLR family members act as entry receptors for yellow fever virus.

As described herein, inhibitors of LDLR (e.g., antibodies, fusion proteins, small molecules) can reduce or prevent a viral infection, including but not limited to orthoflaviviral (e.g. yellow fever (YFV), dengue (DENV), Zika (ZIKV), West Nile (WNV), Japanese encephalitis (JEV), and tick-borne encephalitis (TBEV) viruses) infection. An LDLR inhibiting agent can be any agent that can inhibit LDLR, downregulate LDLR, or knockdown LDLR.

As an example, an LDLR inhibiting agent can inhibit LDLR signaling.

For example, the LDLR inhibiting agent can be an anti-LDLR antibody. As an example, the anti-LDLR antibody can be anti-LRP1 antibody, an antiLRP2 antibody, an anti-LRP3 antibody, an anti-LRP4 antibody, or an anti-LDLR antibody with activity against any combination of LRP1, LRP2, LRP3, or LRP4. Furthermore, the anti-LDLR antibody can be a murine antibody, a humanized murine antibody, or a human antibody.

As another example, the LDLR inhibiting agent can be an anti-LDLR antibody, wherein the anti-LDLR antibody prevents binding of LDLR to its receptor or prevents activation of LDLR and downstream signaling.

As another example, the LDLR inhibiting agent can be a fusion protein. For example, the fusion protein can be a decoy receptor for LDLR. Furthermore, the fusion protein can comprise a mouse or human Fc antibody domain fused to the ectodomain of LDLR.

As another example, an LDLR inhibiting agent can be a decoy receptor, which has been shown to be a potent and specific inhibitor of LDLR signaling.

As another example, an LDLR inhibiting agent can be an inhibitory protein that antagonizes LDLR. For example, the LDLR inhibiting agent can be a viral protein, which has been shown to antagonize LDLR.

As another example, an LDLR inhibiting agent can be a short hairpin RNA (shRNA) or a short interfering RNA (siRNA) targeting LDLR or LRP4.

As another example, an LDLR inhibiting agent can be an sgRNA targeting LDLR or LRP4.

Methods for preparing an LDLR inhibiting agent (e.g., an agent capable of inhibiting LDLR signaling) can comprise construction of a protein/Ab scaffold containing the natural LDLR receptor as an LDLR neutralizing agent; developing inhibitors of the LDLR receptor “down-stream”; or developing inhibitors of the LDLR production “up-stream”.

Inhibiting LDLR can be performed by genetically modifying LDLR in a subject or genetically modifying a subject to reduce or prevent expression of the LDLR gene, such as through the use of CRISPR-Cas9 or analogous technologies, wherein, such modification reduces or prevents a viral infection.

### Chemical Agent:

Examples of LDLR-modulating agents are described herein.

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include-OH. The “hydroxyl” can be optionally substituted.

The terms “halogen” and “halo”, as used herein, unless otherwise indicated, include a chlorine, chloro, CI; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to,-methyl,-ethyl,-n-propyl,-n-butyl,-n-pentyl,-n-hexyl,-n-heptyl and-n-octyl; while branched lower alkyl groups include, but are not limited to,-isopropyl,-sec-butyl,-isobutyl,-tert-butyl,-isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to,-vinyl,-allyl,-1-butenyl,-2-butenyl,-isobutylenyl,-1-pentenyl,-2-pentenyl,-3-methyl-1-butenyl,-2-methyl-2-butenyl,-2,3-dimethyl-2-butenyl, 1-hexyl, 2-hexyl, 3-hexyl,-acetylenyl,-propynyl,-1-butynyl,-2-butynyl,-1-pentynyl,-2-pentynyl, or-3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above, and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2-O-2,5-dimethylhexyl, —O-3,5-dimethylhexyl, —O-2,4dimethylpentyl, methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to,-cyclopropyl,-cyclobutyl,-cyclopentyl,-cyclopentadienyl,-cyclohexyl,-cyclohexenyl,-1,3-cyclohexadienyl,-1,4-cyclohexadienyl,-cycloheptyl,-1,3-cycloheptadienyl,-1,3,5-cycloheptatrienyl,-cyclooctyl, and-cyclooctadienyl. The term “cycloalkyl” also can include-lower alkyl-cycloalkyl, wherein lower alkyl and cycloalkyl are as defined herein. Examples of-lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or—CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65 °C in the salt conditions of a 6×SSC, then the two sequences will not hybridize. On the other hand, if the melting temperature is above 65 °C in the same salt conditions, then the sequences will hybridize. In general, the melting temperature for any hybridized DNA: DNA sequence can be determined using the following formula: Tₘ=81.5 °C+16.6 (log₁₀ [Na⁺])+0.41 (fraction G/C content)−0.63 (% formamide)−(600/l). Furthermore, the Tₘ of a DNA:DNA hybrid is decreased by 1-1.5 °C for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, LDLR signals can be modulated (e.g., reduced, eliminated, or enhanced) using genome editing. Processes for genome editing are well known; see e.g. Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of LDLR by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for viral therapy to target cells by the removal of LDLR signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0 °C and about 60 °C, for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a viral infection in a subject in need of administration of a therapeutically effective amount of an LDLR-modulating agent, so as to inhibit viral entry into the cells of a subject in need.

Methods described herein are generally performed on a subject in need thereof. A subject in need of the therapeutic methods described herein can be a subject having, diagnosed with, suspected of having, or at risk for developing a viral infection. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an LDLR-modulating agent is, for example, an amount that would cause the desired therapeutic effect in a subject while minimizing undesired side effects. In various embodiments, an effective amount of an LDLR-modulating agent described herein can substantially inhibit a viral infection, slow the progress of a viral infection, or limit the development of a viral infection.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an LDLR-modulating agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a viral infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an LDLR-modulating agent can occur as a single event or over a time course of treatment. For example, an LDLR-modulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a viral infection.

An LDLR-modulating agent can be administered simultaneously or sequentially with another agent, such as an antibiotic, an anti-inflammatory, or another agent. For example, an LDLR-modulating agent can be administered simultaneously with another agent, such as an antibiotic or an anti-inflammatory. Simultaneous administration can occur through administration of separate compositions, each containing one or more of an LDLR-modulating agent, an antibiotic, an anti-inflammatory, or another agent. Simultaneous administration can occur through administration of one composition containing two or more of an LDLR-modulating agent, an antibiotic, an anti-inflammatory, or another agent. An LDLR-modulating agent can be administered sequentially with an antibiotic, an anti-inflammatory, or another agent. For example, an LDLR-modulating agent can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about-2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to an LDLR-modulating agent, delivery vehicles, solubilizing agents, and sterile packaging. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Multiple Ldlr Family Members Act as Entry Receptors for Yellow Fever Virus

Orthoflaviviruses are insect-transmitted RNA viruses with epidemic potential that cause morbidity and mortality on a global scale. Infection by yellow fever virus (YFV), the prototype orthoflavivirus, induces a febrile syndrome in humans that can progress rapidly to liver failure, hemorrhage, and death. Despite decades of study, the entry receptors for YFV remain unknown.

Using a surface protein-targeted CRISPR-Cas9 library and a loss-of-infection screen in HAP1 cells, low-density lipoprotein receptor (LDLR)-related protein 4 (LRP4) was identified as a candidate entry receptor for YFV. Genetic ablation of LRP4 impaired YFV infection in cell culture, and reciprocally, complementation or ectopic expression of LRP4 increased infection. Related viruses in the YFV antigenic complex (e.g., Banzi, Uganda S, and Wesselsbron viruses), but not dengue serocomplex viruses, also showed LRP4-dependent infection. LRP4 promoted YFV attachment to and internalization in cells through LDLR type A (LA) domain binding to domain III of the YFV envelope (E) protein. Soluble LRP4-Fc decoy receptors neutralized YFV infection in cell culture and reduced viral burden in vivo, and mice with hypomorphic Lrp4 allele sustained less YFV infection than wild-type mice. Because residual YFV infection was observed in LRP4-deficient cells, a targeted screen was performed to determine whether other LDLR family members can promote YFV entry. This approach identified LRP1 and VLDLR as additional receptors for YFV infection in cell culture. Both LRP1-Fc and VLDLR-Fc decoys bound to and neutralized YFV in vitro, and an LRP1-Fc decoy molecule also inhibited YFV infection in mice. The findings establish a role for multiple LDLR family members in YFV entry and infection, which has implications for receptor usage and countermeasure development against multiple emerging orthoflaviviruses.

### Results

There are more than 70 arthropod-transmitted orthoflaviviruses, including YFV, Dengue (DENV), Zika (ZIKV), West Nile (WNV) and Japanese encephalitis (JEV). Orthoflaviviruses have an approximately 11-kb positive-sense RNA genome that encodes three structural (C, prM and E) and seven non-structural (NS1, NS2A, NS2B, NS3, NS4A, NS4B and NS5) proteins. Infections in humans can cause severe disease including multi-organ failure, hemorrhage, meningitis, encephalitis and congenital malformations.

YFV is transmitted to humans principally by Aedes aegypti mosquitoes and replicates in cells of the liver, kidney, bloodstream and, less frequently, the brain. Approximately 30% of humans with YFV infection develop acute hepatic disease, and these cases have a high (approximately 50%) fatality rate. Historically, YFV caused epidemics that threatened millions of people. The introduction of vector control measures and a live-attenuated YFV-17D vaccine markedly reduced transmission and disease. Nonetheless, YFV remains a global health threat, particularly in tropical and subtropical regions of Africa and South America, due to vaccine shortages and sporadic vaccination coverage. To date, there is no specific antiviral treatment for YFV or any other Orthoflavivirus. The potential for urban epidemic outbreaks with a direct human-mosquito-human transmission cycle and its high case-fatality rate make yellow fever a persistent threat.

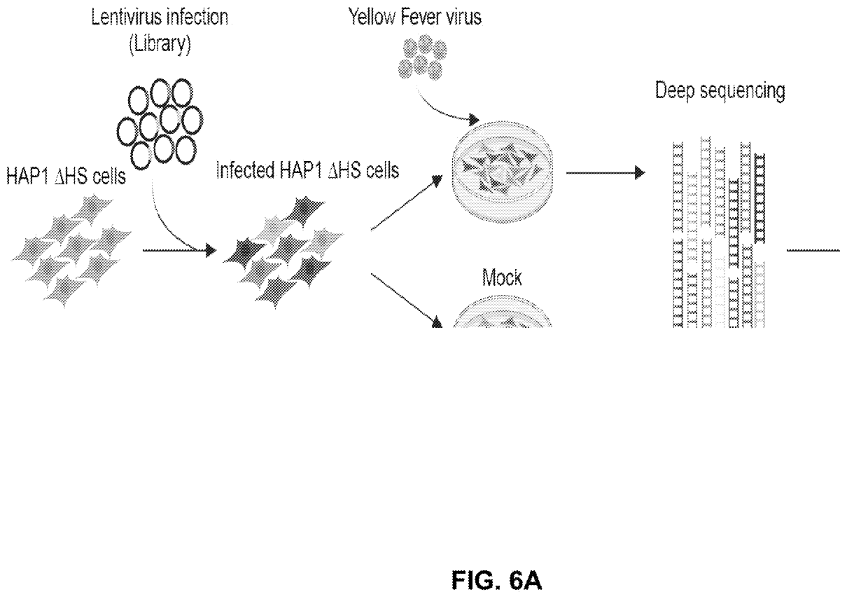

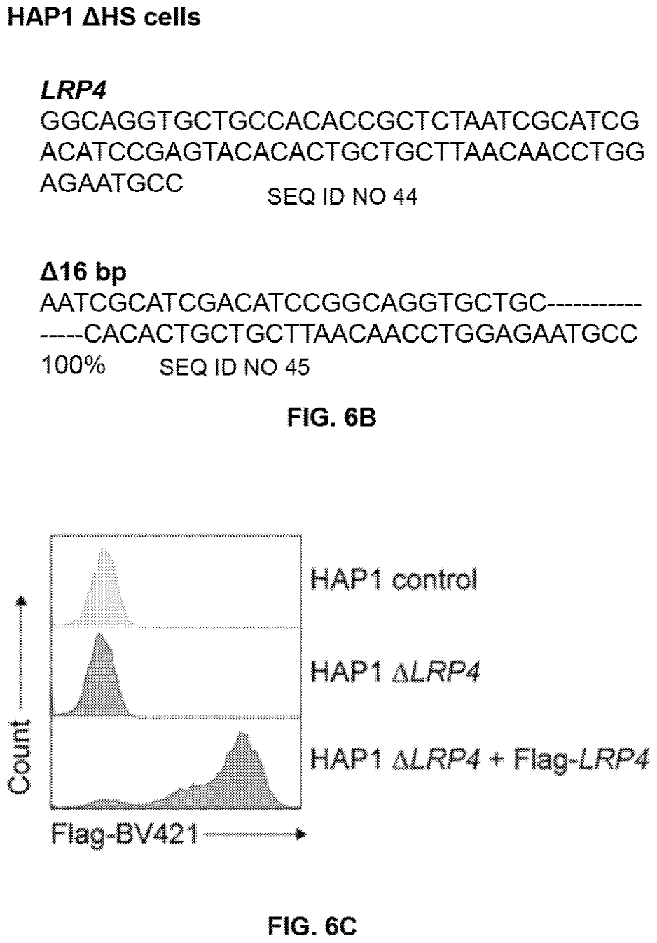

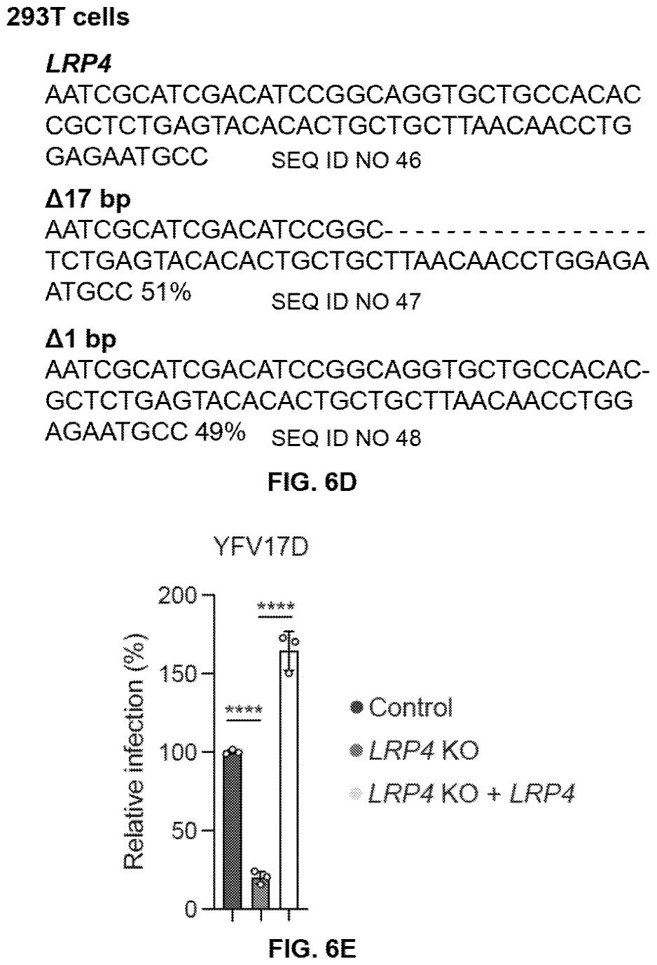

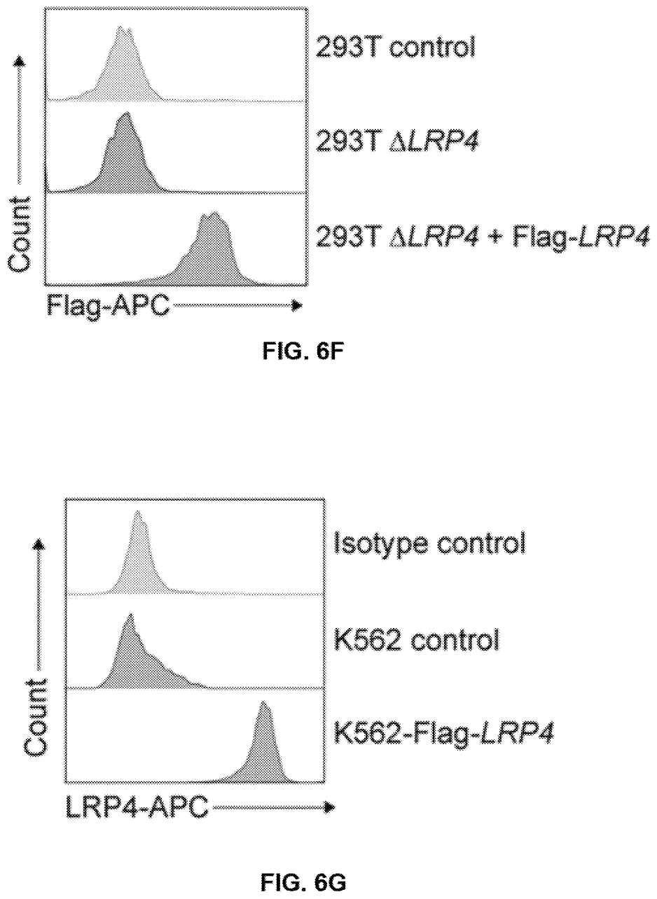

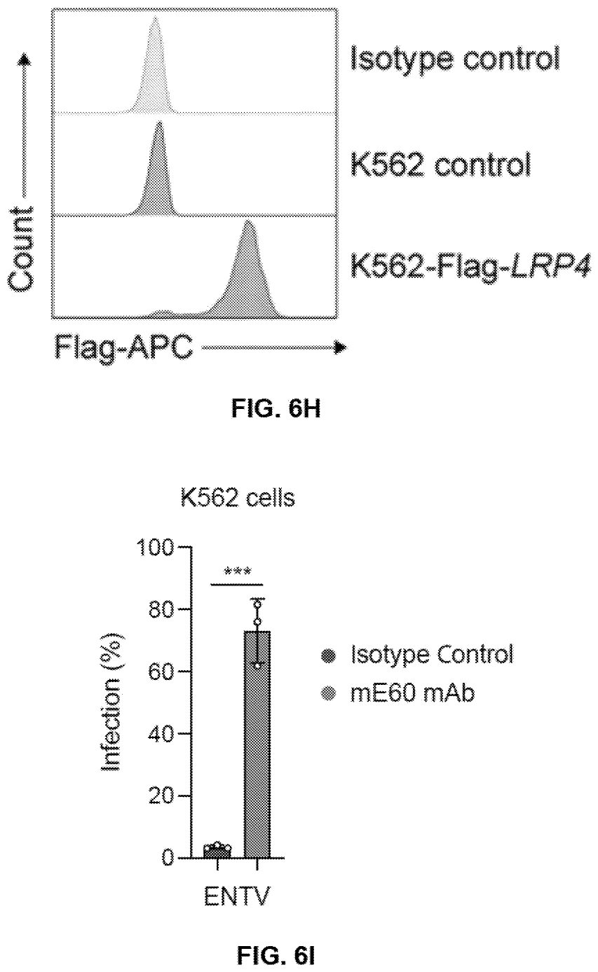

The identification of viral-entry receptors is important for understanding tropism and developing targeted antiviral therapies. Compared with the distantly related alphaviruses, for which several receptors have been identified through CRISPR-Cas9-based screens, less is known about the factors that mediate attachment and internalization of orthoflaviviruses. Some cell surface proteins including T cell immunoglobulin and mucin domain 1 (TIM-1), dendritic-cell-specific intercellular adhesion molecule-3-grabbing non-integrin (DC-SIGN), CD300A and the receptor tyrosine kinase AXL, are implicated in entry of some orthoflaviviruses (such as DENV, ZIKV, JEV and WNV). However, their loss in vivo in mice or cell culture has limited effects on infection and pathogenesis, suggesting functional redundancy or subordinate roles, and few of these proteins appear to influence YFV entry. To identify candidate cellular receptors for YFV, a lentivirus library of 4,630 single guide RNAs (sgRNAs) was generated targeting 1,146 human genes that encode proteins predicted to localize to the plasma membrane (FIG. 6A) for CRISPR-Cas9 screening. As some tissue culture passaged orthoflaviviruses can use heparan sulfate (HS) for initial cell attachment, the screen was designed in human haploid HAP1 leukemia cells lacking both SLC35B2 and XYLT2 (hereafter, HAP1 ΔHS cells). SLC35B2 is a transporter protein that is required for sulfation of HS, and XYLT2 is a key enzyme involved in HS biosynthesis.

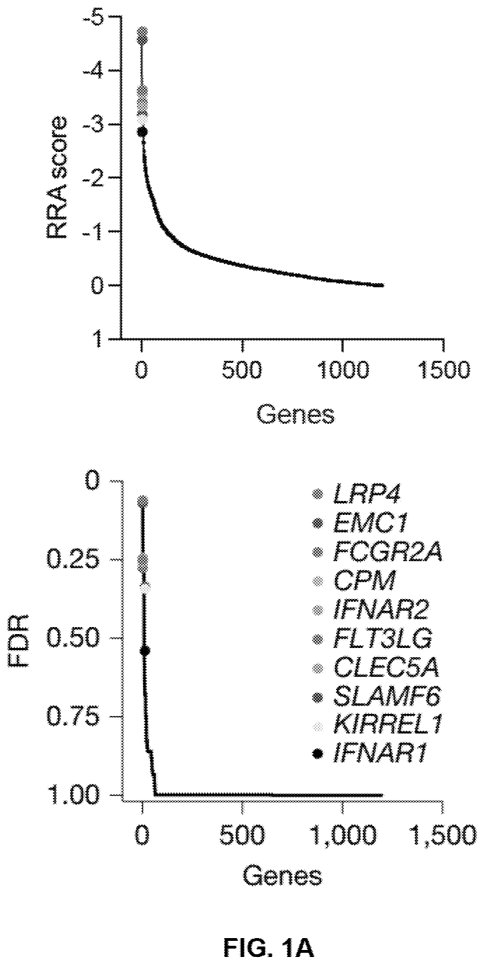

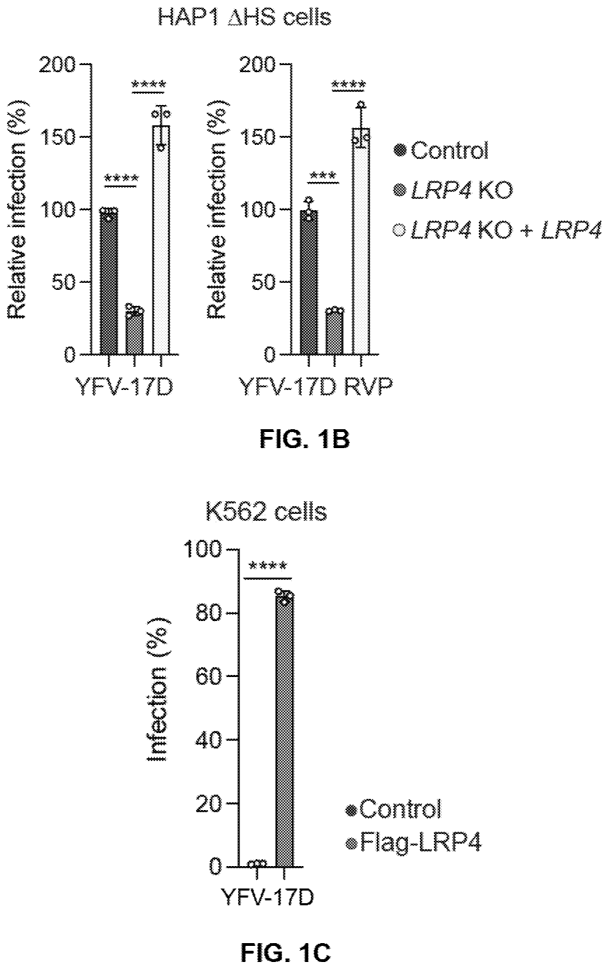

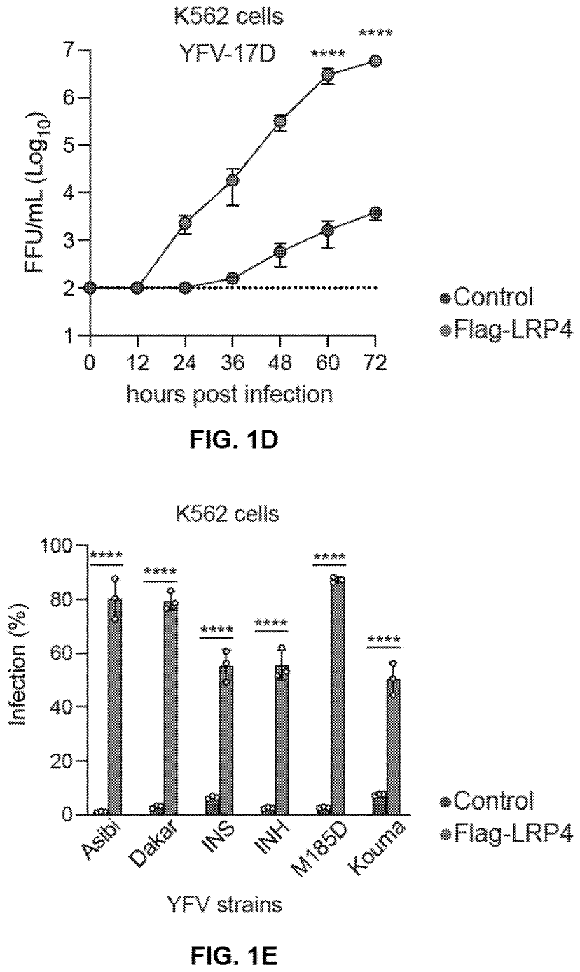

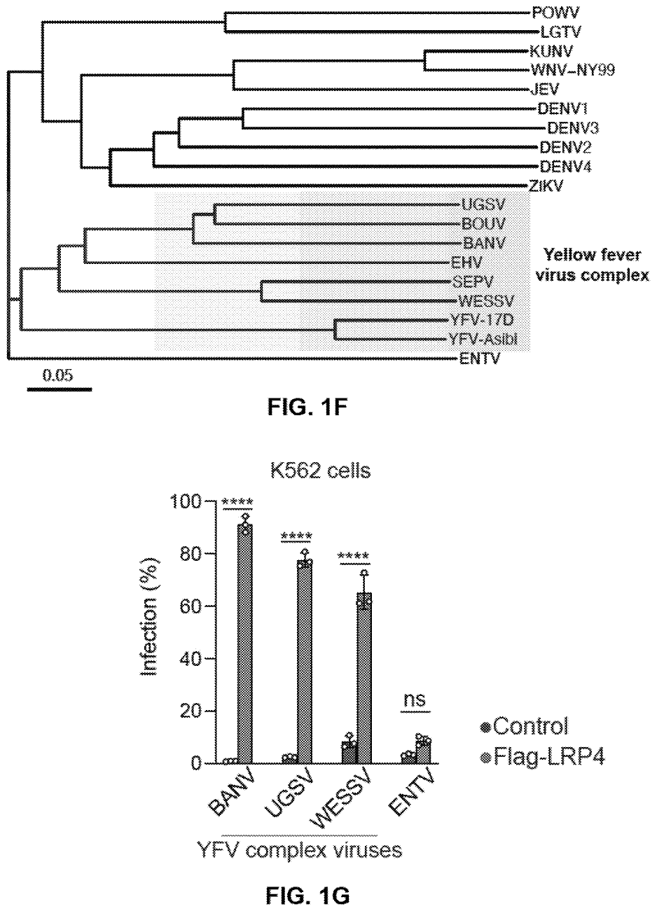

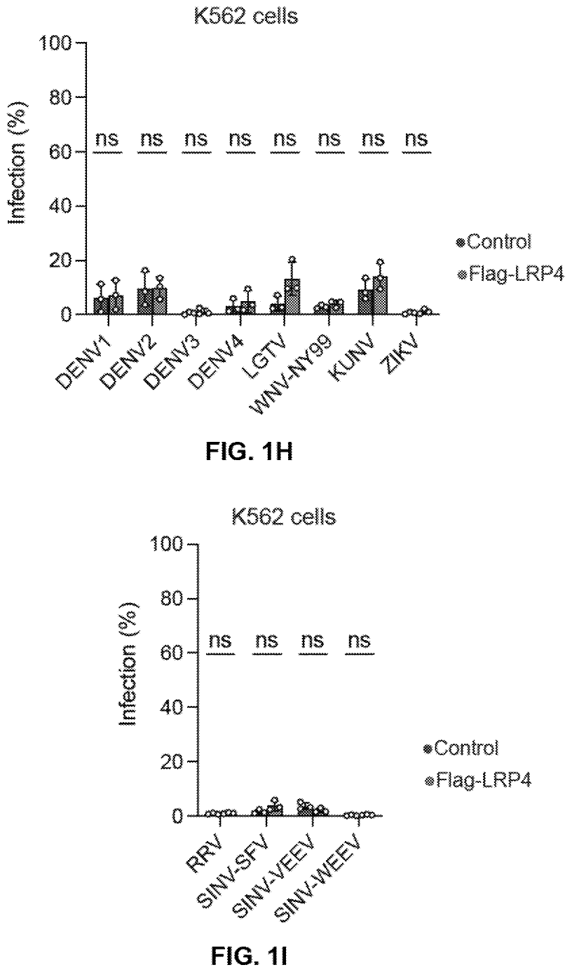

The screen was performed at biosafety containment level 2 with the attenuated YFV-17D vaccine strain. HAP1 ΔHS library cells were inoculated with YFV-17D at a multiplicity of infection (MOI) of 10 and were monitored for cell survival. After expanding the few surviving cells, genomic DNA was collected for sequencing to identify sgRNA enrichment. The screen identified LRP4 as the top hit (FIG. 1A, FIG. 6A). LRP4 is a single-pass, type I transmembrane protein and a member of the LDLR family that mediates endocytosis of lipoproteins and other ligands. EMC1 also was enriched in the screen (FIG. 1A). EMC1 is a subunit of the endoplasmic reticulum (ER) membrane complex (EMC) and has key roles in protein insertion into the ER. EMC genes were reported previously to be essential host factors for replication of other orthoflaviviruses including DENV, ZIKV, WNV and YFV.

This study focused on validating LRP4 as a key molecule required for YFV infection. Clonal ΔHS ΔLRP4 HAP1 cells showed reduced YFV-17D infection (FIG. 1B, FIG. 6B), and complementation of LRP4 restored infectivity (FIG. 1B, FIG. 6C). A similar phenotype was observed with single-cycle YFV reporter virus particles (YFV-17D RVPs) that cannot spread beyond the initial infected cell (FIG. 1B). A loss of LRP4 expression in HEK293T cells also was associated with reduced YFV-17D infection (FIG. 6D, FIG. 6E, FIG. 6F). Next, it was assessed whether ectopic expression of LRP4 could promote YFV infection. K562 erythroleukemic cells were used and showed limited permissiveness to Orthoflavivirus infection in the absence of an exogenously provided entry pathway, such as Fc γ receptors (FcγR) and antibody-dependent enhancement (ADE). A N-terminal Flag-tagged human LRP4 lentivirus construct was designed and transduced into K562 cells so expression could be monitored. Ectopic expression of LRP4 resulted in increased YFV-17D infection compared with in the control, parental K562 cells (FIG. 1C, FIG. 6G, FIG. 6H). In multi-step growth experiments, higher YFV-17D yields were measured in the supernatants of cells expressing LRP4 compared with those from control cells (FIG. 1D).

To test whether LRP4 expression could support infection of other YFV strains, K562 control and K562-LRP4 cells were inoculated with the parental, virulent YFV-Asibi (from which YFV-17D was derived) and five additional pathogenic strains isolated in Africa (Dakar, Kouma and M185D) or South America (INH and INS). Ectopic expression of LRP4 promoted infection of all of the YFV strains that were tested (FIG. 1E). Also evaluated was whether related viruses in the YFV antigenic complex could use LRP4 for infection. Infection of Banzi (BANV), Uganda S (UGSV) and Wesselsbron (WESSV) viruses was increased in K562 cells expressing LRP4 compared with the control cells, although this phenotype was not observed with the more distantly related Entebbe bat virus (ENTV) (FIG. 1F, FIG. 1G). When ENTV was allowed to infect K562 cells through an ADE pathway, viral-antigen-positive cells were observed (FIG. 6I), suggesting that its infection was limited by the lack of an effective entry pathway, which LRP4 did not provide. Infection by orthoflaviviruses from other antigenic complexes, including DENV, Langat virus (LGTV), WNV and ZIKV was not supported by LRP4 expression (FIG. 1H). Although several other LDLR family members support alphavirus entry, LRP4 expression in K562 cells did not promote infection of Ross River virus (RRV) or chimeric alphaviruses expressing the structural genes of Western equine encephalitis (WEEV), Venezuelan equine encephalitis (VEEV) and Semliki Forest (SFV) viruses (FIG. 1I).

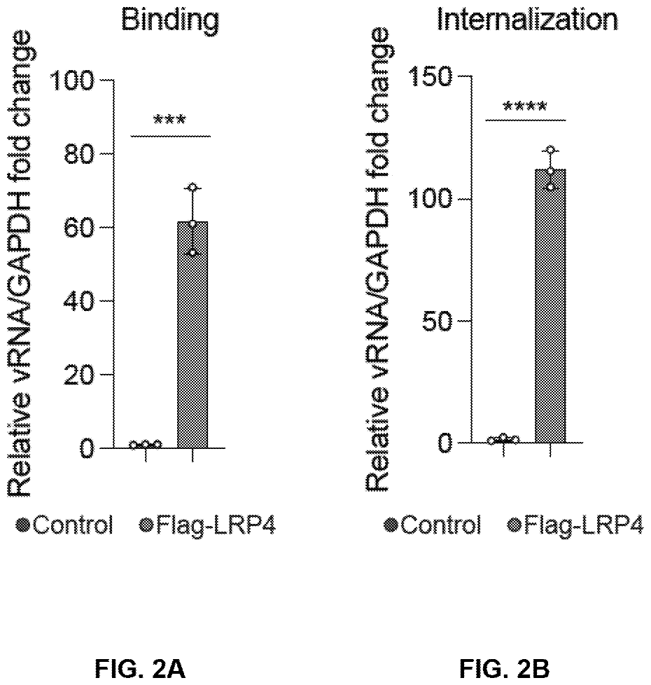

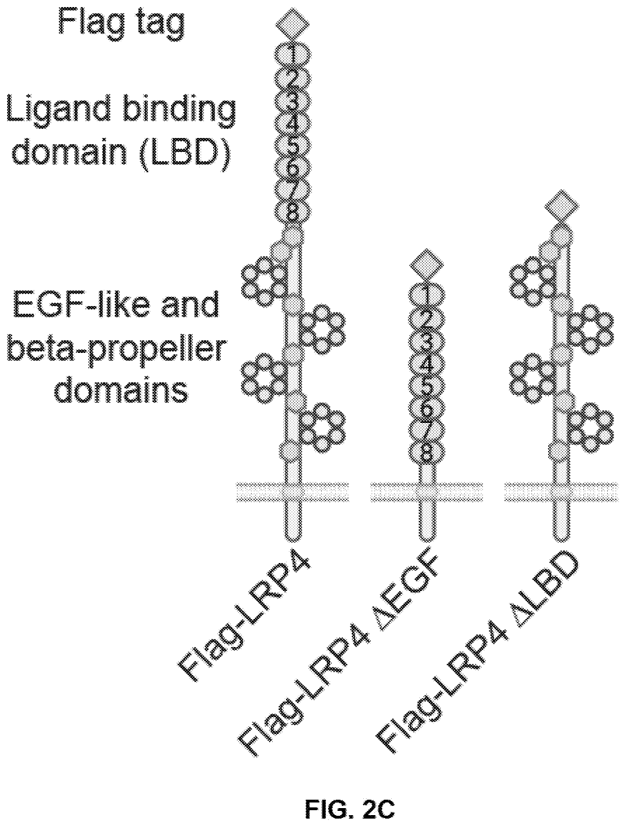

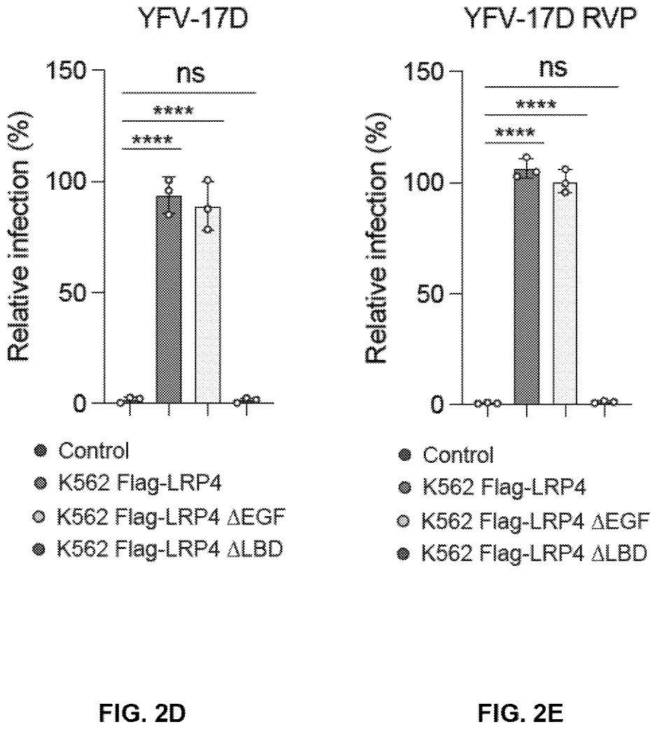

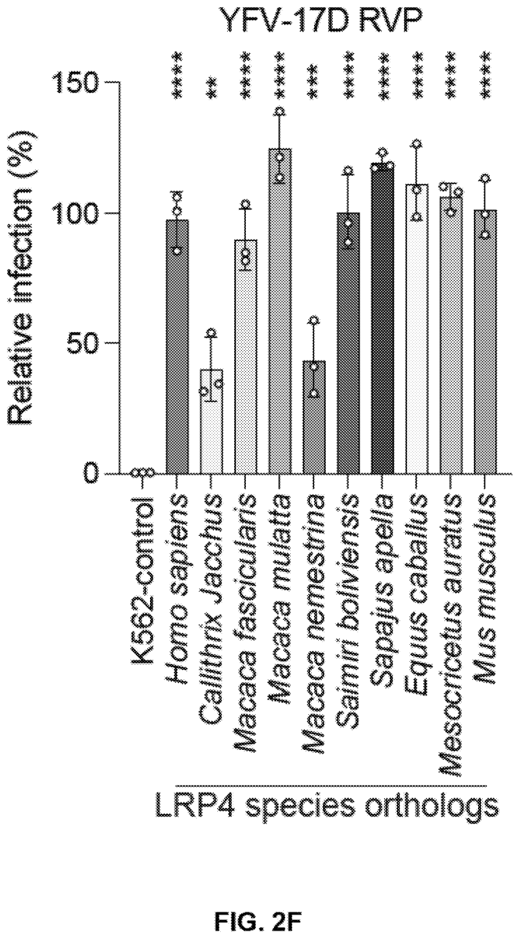

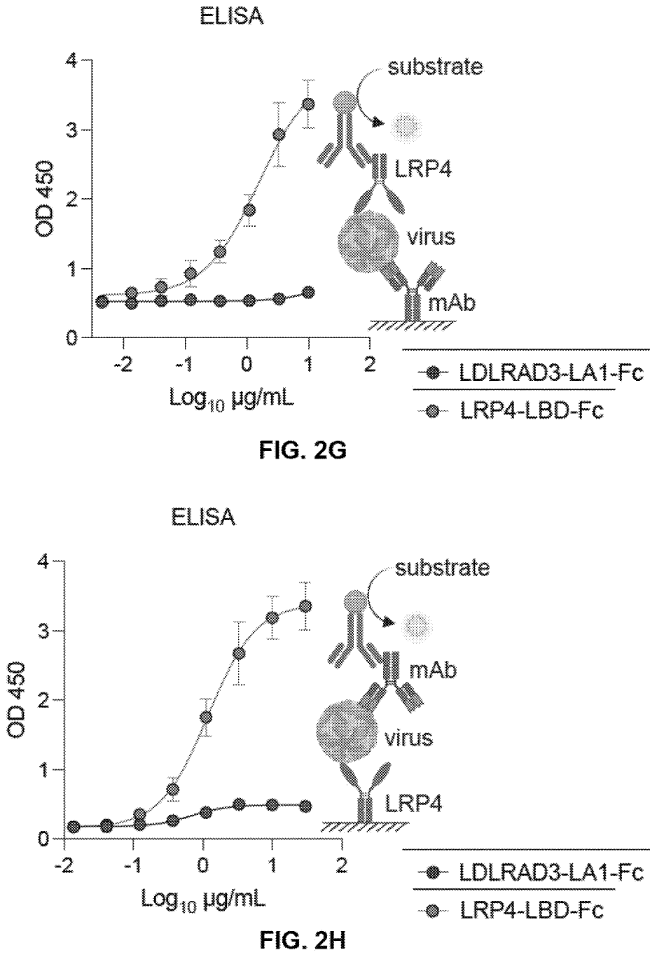

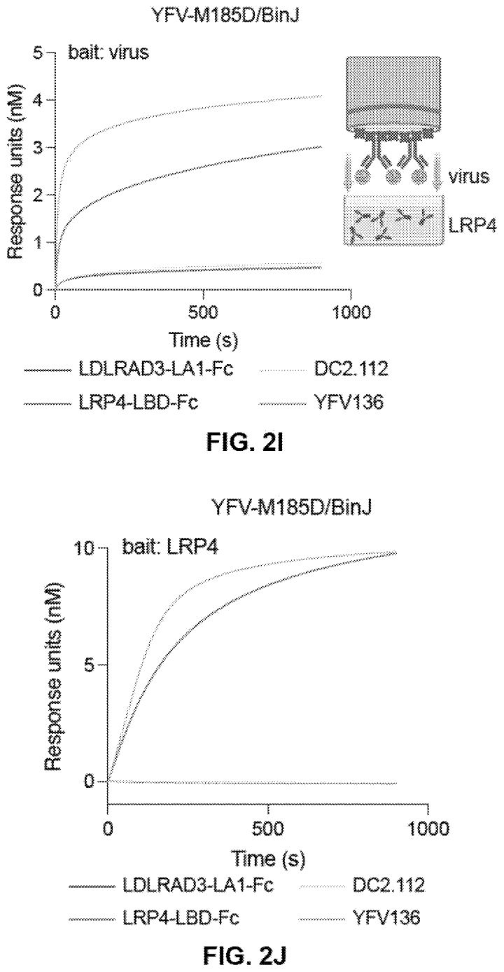

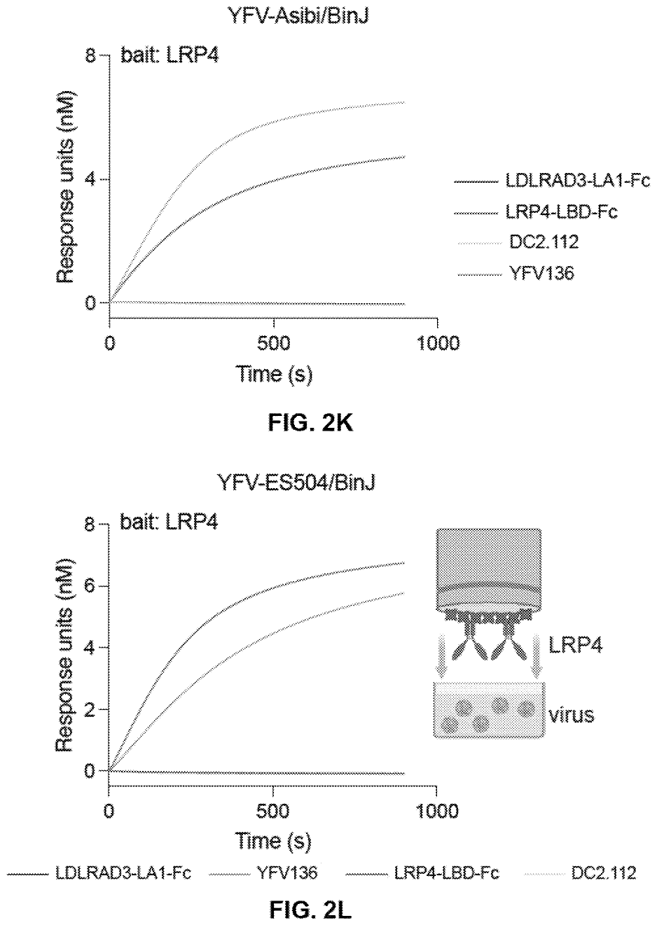

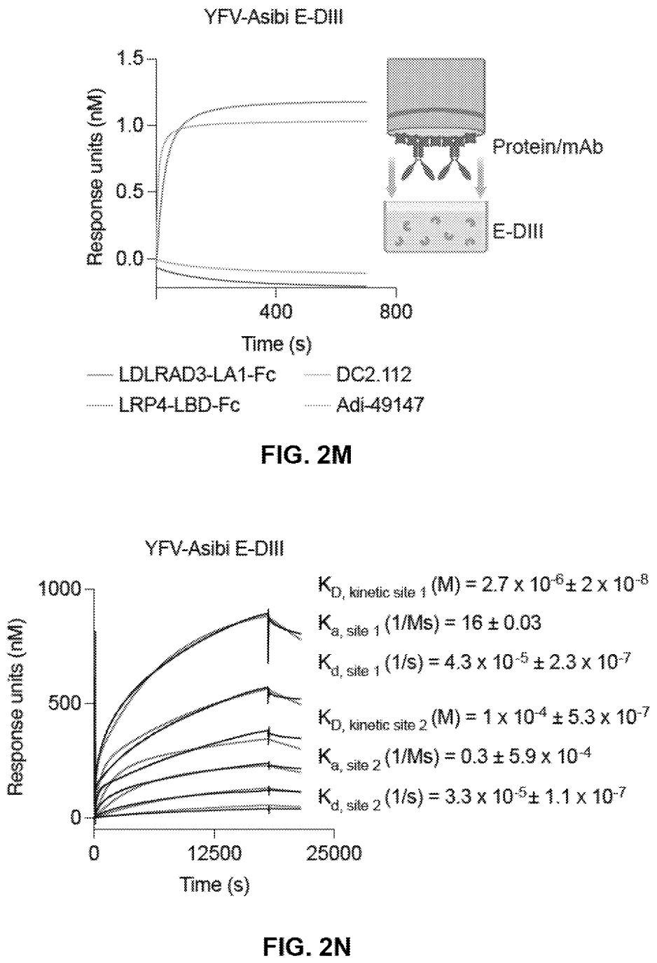

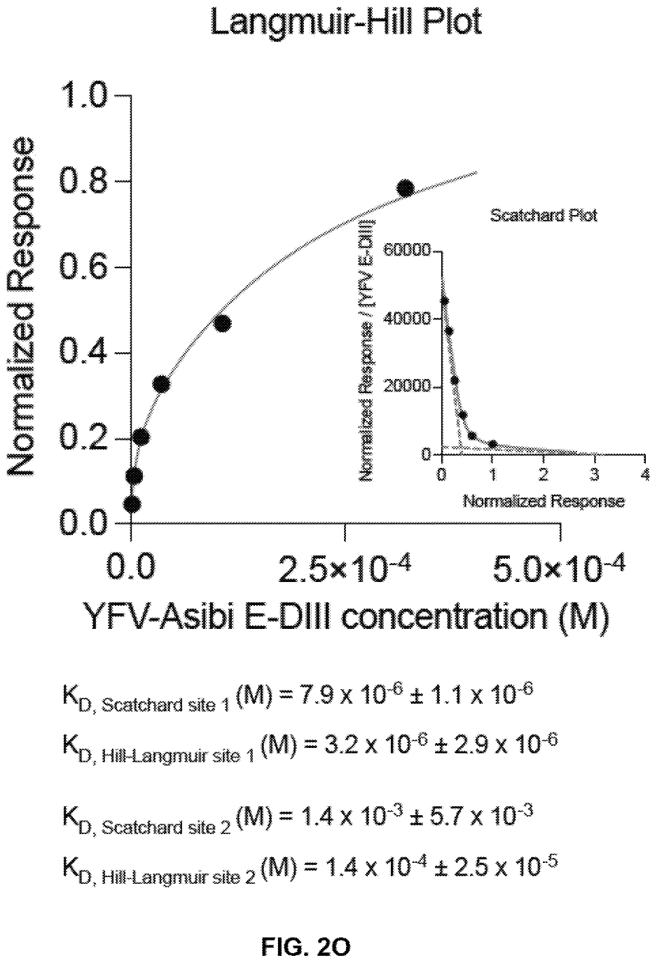

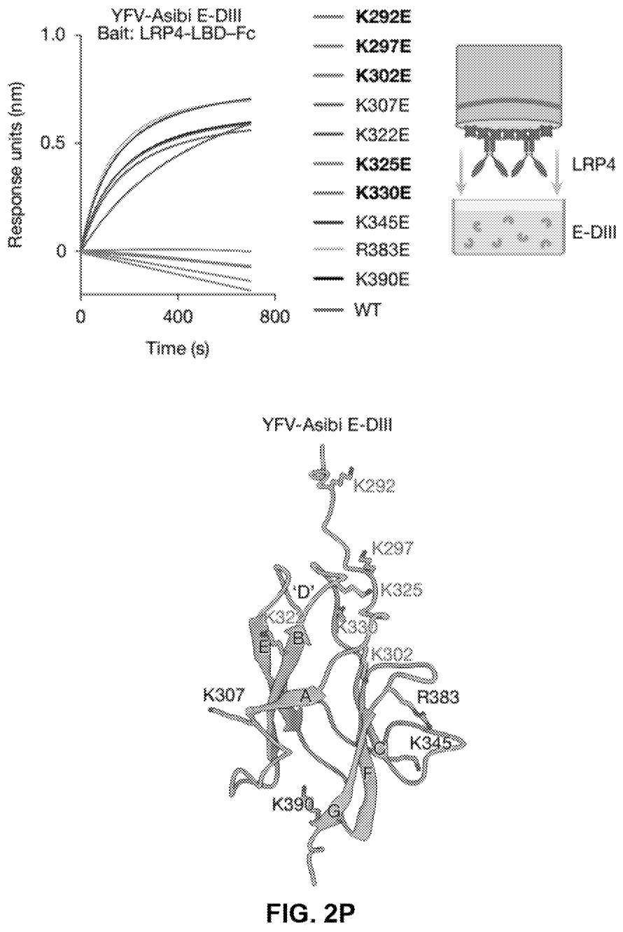

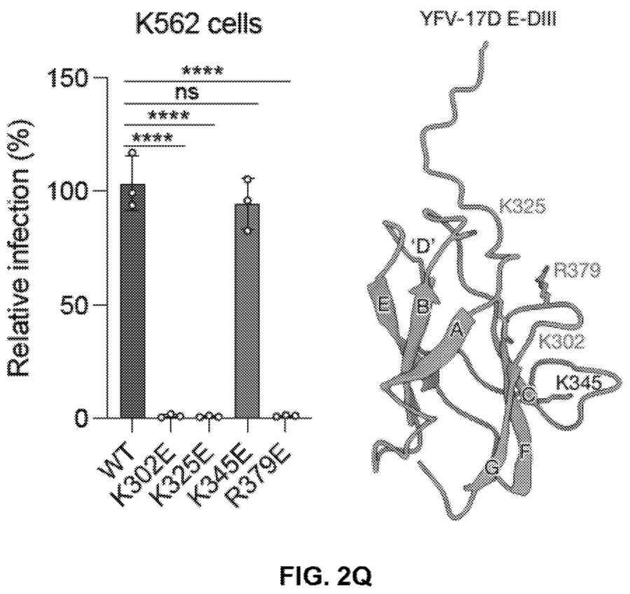

As LRP4 is expressed on the plasma membrane, it was hypothesized that it might be required for efficient YFV attachment and/or entry. To test this idea, a virus-cell binding assay was performed in which YFV-17D was incubated with K562 cells on ice. After extensive rinsing to remove unbound viruses, binding to cells was measured using quantitative PCR with reverse transcription (RT-qPCR). Compared with the control K562 cells, LRP4-expressing K562 cells supported greater binding of YFV-17D (FIG. 2A). After virus internalization was allowed to occur by transitioning the cells to 37 °C and then removing surface-adsorbed virus with proteinase K and RNase treatment, more YFV-17D RNA was detected within LRP4-expressing K562 cells than in control cells (FIG. 2B). Thus, LRP4 expression can promote YFV attachment to and internalization into host cells.

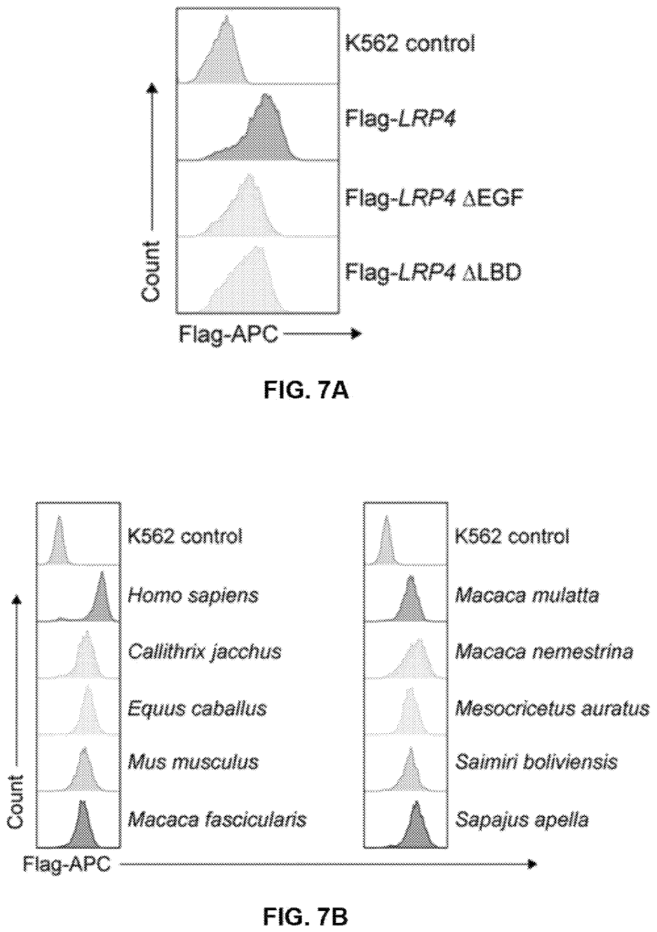

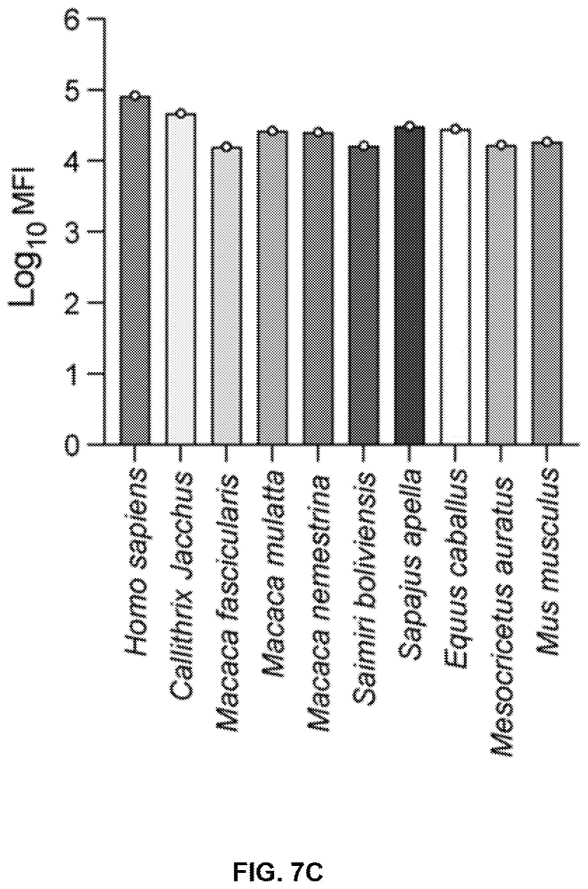

The extracellular region of LRP4 contains an N-terminal ligand-binding domain (LBD) comprising LA repeats, and a cell-proximal region comprising EGF-like and β-propeller domains (FIG. 2C). Owing to its membrane distal location, it was hypothesized that the LBD of LRP4 interacts with YFV. To test this idea, variants of LRP4 were engineered that lack the EGF-like and β-propeller domains or LBD (FIG. 2C). Although all N-terminally Flag-tagged proteins were expressed efficiently on the surface of K562 cells (FIG. 7A), only those containing the LBD supported YFV-17D virus or RVP infection (FIG. 2D, FIG. 2E). YFV also has a sylvatic transmission cycle between arboreal mosquitoes and non-human primate (NHP) reservoirs. As the LBD of LRP4 is conserved (around 83 to 99%) among mammals, it was tested whether orthologues could promote YFV infection by swapping the human LRP4 LBD with those of other species. K562 cells expressing LRP4 LBDs from different NHP species (Callithrix jacchus, Macaca fascicularis, Macaca mulatta, Macaca nemestrina, Saimiri boliviensis and Sapajus apella), horses (Equus caballus), mice (Mus musculus) or hamsters (Mesocricetus auratus) all supported YFV-17D RVP infection to varying degrees (FIG. 2F, FIG. 7B, FIG. 7C). M. fascicularis and S. boliviensis LRP4 showed slightly lower expression levels yet still supported YFV-17D RVP infection (FIG. 2F, FIG. 7B, FIG. 7C). By contrast, C. jacchus and M. nemestrina LRP4 supported less YFV-17D RVP infection, although their surface expression was equivalent to others (FIG. 2F, FIG. 7B, FIG. 7C). Although further studies are required, the lower YFV-17D infection seen with C. jacchus and M. nemestrina could be due to their few sequence polymorphisms.

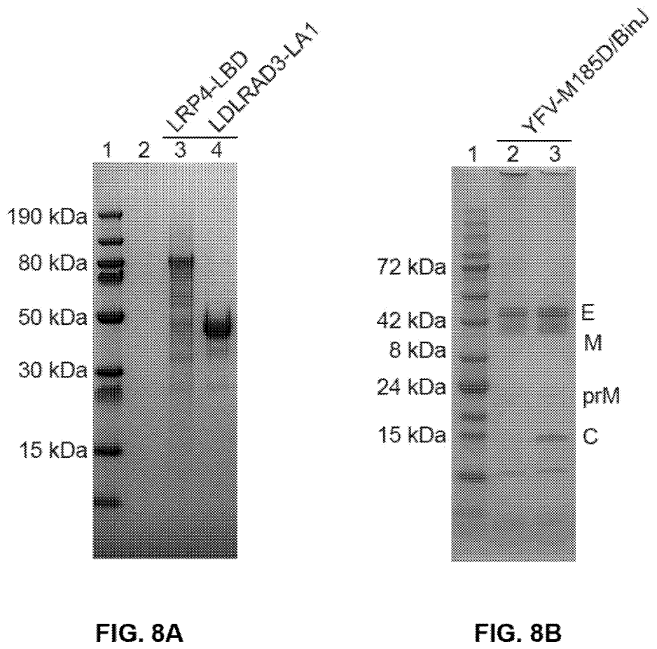

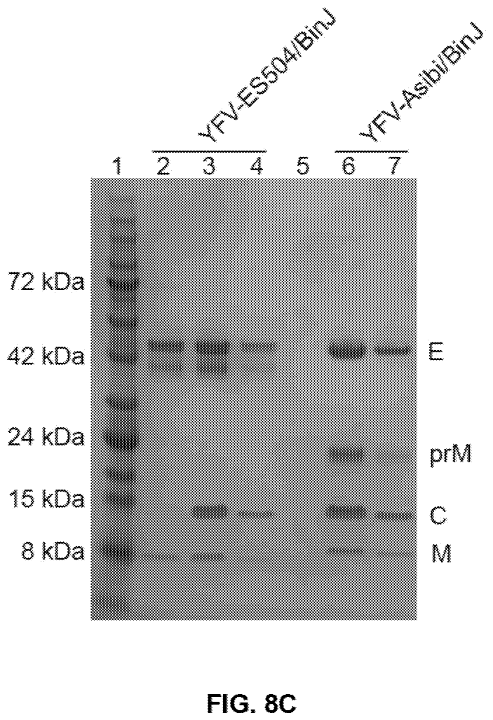

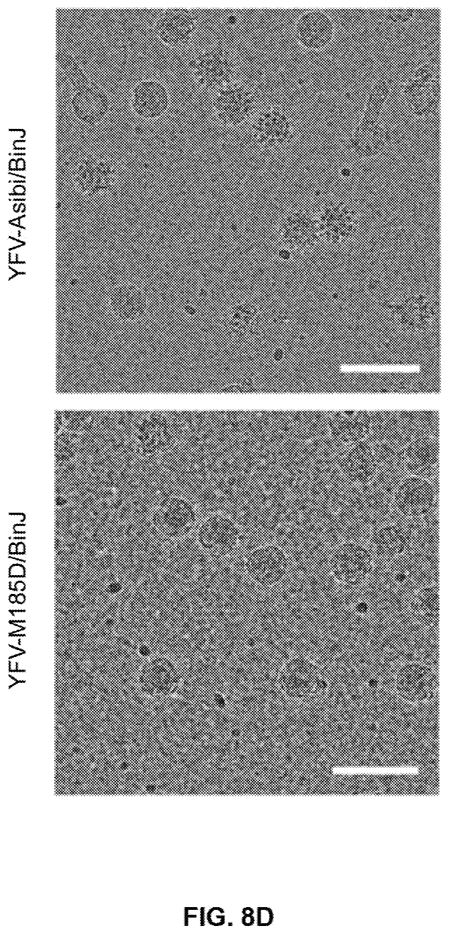

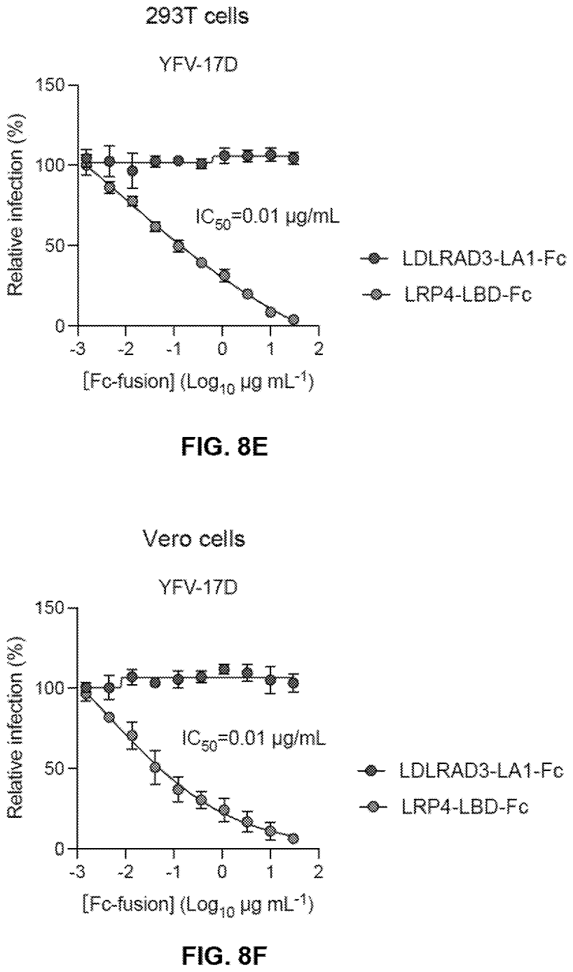

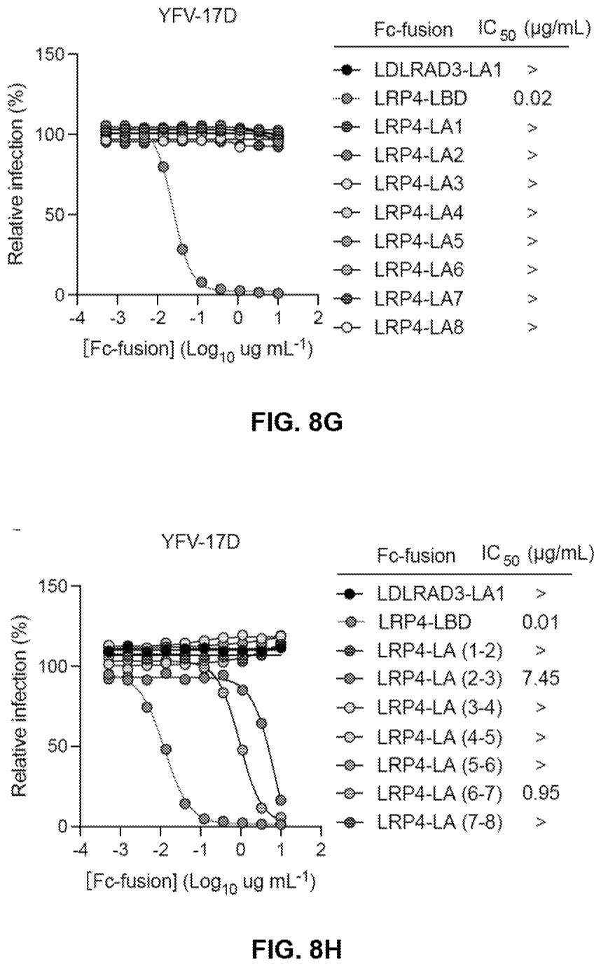

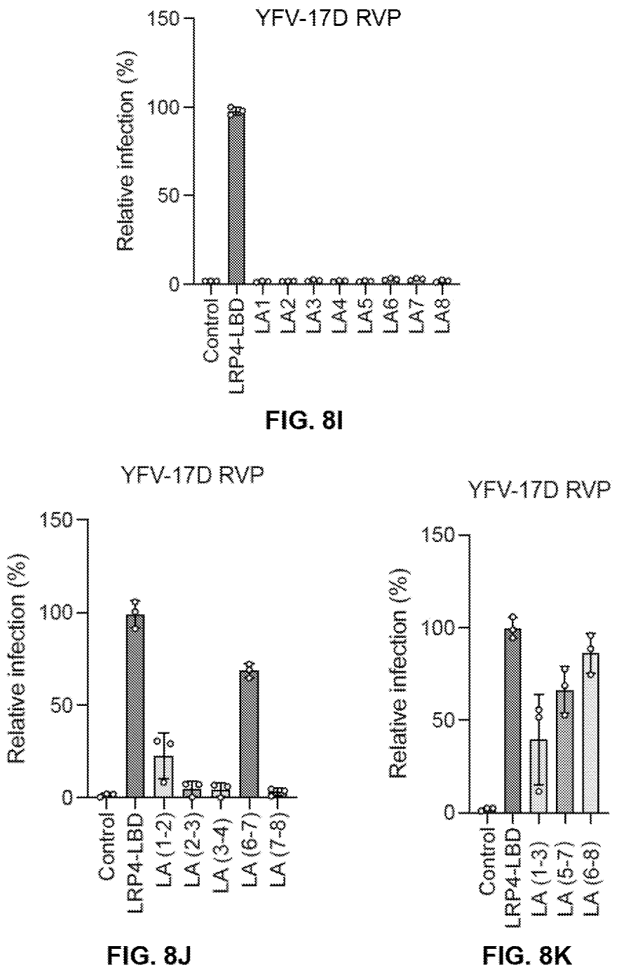

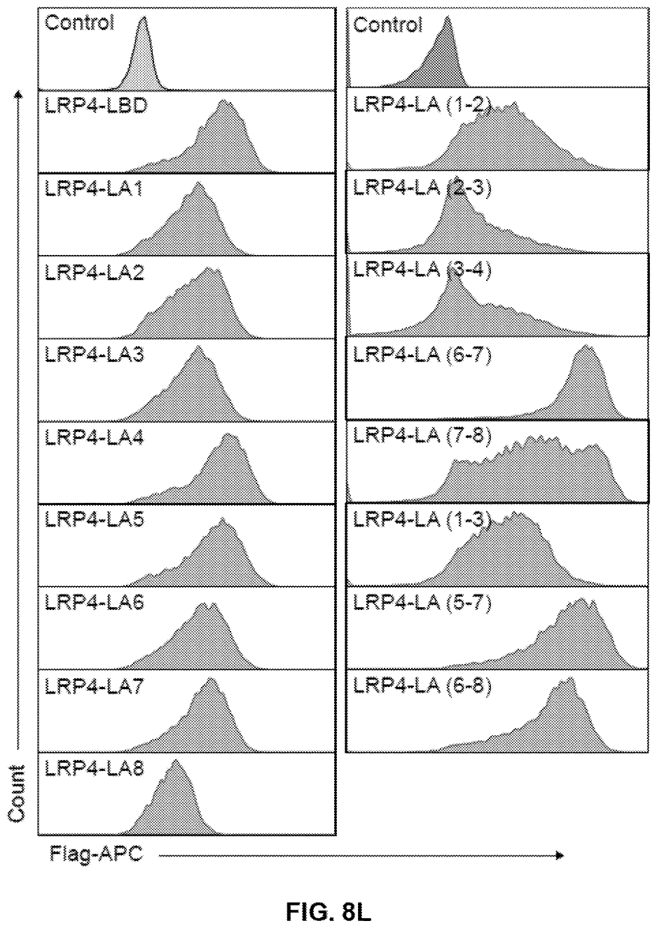

Next, it was evaluated whether the LRP4 LBD could directly interact with YFV by assessing binding of a recombinant Fc fusion protein (LRP4-LBD-Fc). In reciprocally designed enzyme-linked immunosorbent assay (ELISA) experiments, in which YFV-17D virions were captured in the solid phase or added in solution, direct binding was observed to LRP4-LBD-Fc but not to LDLRAD3-LA1-Fc protein—a fusion protein derived from a different LDLR superfamily member (FIG. 2G, FIG. 2H, FIG. 8A). To corroborate these findings, biolayer interferometry (BLI) experiments were performed. Pathogenic YFV strains require BSL3 containment, however, a chimeric biosafety containment level 2 virus platform in which the structural prM and E genes of YFV replace those of Binjari virus (BinJV), an insect-restricted orthoflavivirus. Previous cryo-electron microscopy analyses established that chimeric BinJV recapitulates the assembly of the Orthoflavivirus virions encoded by the structural genes. YFV-M185D/BinJ, YFV-Asibi/BinJ and YFV-ES504/BinJ chimeric viruses were generated for BLI binding experiments (FIG. 8B, FIG. 8C, FIG. 8D). Using a biotinylated anti-YFV E protein monoclonal antibody (mAb YFV-136), YFV virions were immobilized purified on biosensors and evaluated binding of LRP4-LBD-Fc protein. Whereas LRP4-LBD-Fc protein and YFV-136 bound to YFV virions, LDLRAD3-LA1-Fc protein and DC2.112 mAb, a negative control anti-alphavirus E1 mAb, did not (FIG. 2I). In a reciprocally designed experiment, immobilized LRP4-LBD-Fc or YFV-136 mAb bound to YFV-M185D/BinJ, YFV-Asibi/BinJ and YFV-ES504/BinJ virions in solution, but LDLRAD3-LA1-Fc protein and DC2.112 mAb did not (FIG. 2J, FIG. 2K, FIG. 2L).

Domain III of the E protein (E-DIII) has been hypothesized to interact with cell surface proteins to facilitate Orthoflavivirus entry. To investigate whether LRP4 interacts with YFV E-DIII, in a first set of BLI binding experiments, trimeric YFV E-DIII fused to T4 fibritin was used to mimic the icosahedral arrangement of DIll on the virion. Both LRP4-LBD-Fc and the YFV-E-DIII-specific mAb Adi-49147 bound to YFV E-DIII, whereas LDLRAD3-LA1-Fc protein or the negative control mAb DC2.112 did not (FIG. 2M). Next, using surface plasmon resonance, the binding affinity of monomeric YFV E-DIII was determined for immobilized LRP4-LBD-Fc. The binding data were best fit by a two-site model with K_{D} values at sites 1 and 2 of 2.7 UM and 0.1 mM, respectively, by non-equilibrium, kinetic binding analysis (FIG. 2N). The two-site model was corroborated by equilibrium binding analyses, as Scatchard analysis showed a nonlinear response with K_{D} values at sites 1 and 2 of 7.9 UM and 1.4 mM, respectively. By contrast, Hill-Langmuir analysis measured the K_{D} values at sites 1 and 2 as 3.2 UM and 0.14 mM, respectively (FIG. 2O).

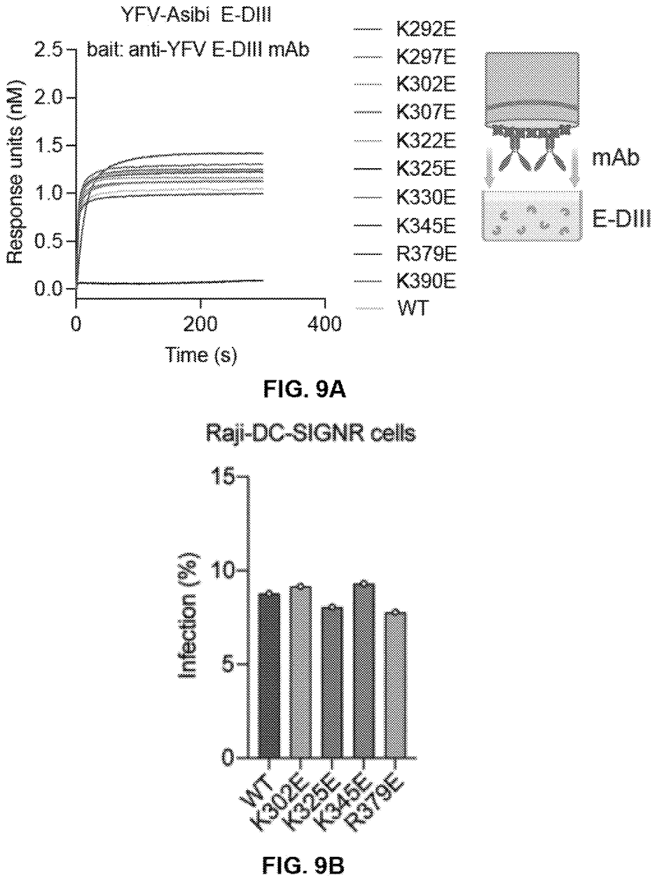

As previous experiments established that positively charged residues in alphavirus envelope proteins interact with VLDLR, a related LDLR family member, it was investigated whether mutation of positively charged residues in E-DIll of YFV would affect binding to LRP4. Indeed, mutation of several solvent-accessible positively charged residues in YFV-Asibi E-DIII (such as K292E, K297E, K302E, K325E or K330E) resulted in a loss of binding to LRP4-LBD-Fc protein, but not to the mAb Adi-49147 (except for K325E) (FIG. 2P, FIG. 9A). To corroborate these results, YFV-17D RVPs were generated in which positively charged (arginine (R) or lysine (K)) residues in E-DIII were changed to negatively charged (glutamic acid (E)) residues. Notably, substitutions in E-DIII at K302E, K325E and R379E, but not K345E, abolished YFV-17D RVP infectivity in LRP4-expressing K562 cells (FIG. 2Q). However, the loss of infectivity conferred by these mutations was not observed in Raji cells expressing DC-SIGNR, of which interaction relies on N-linked glycans in the prM or E glycoproteins (FIG. 9B).

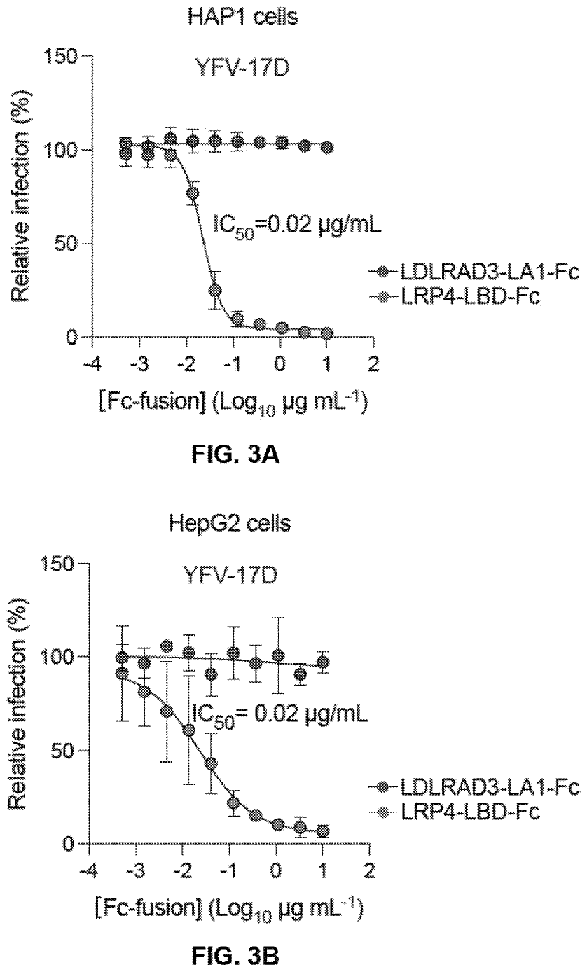

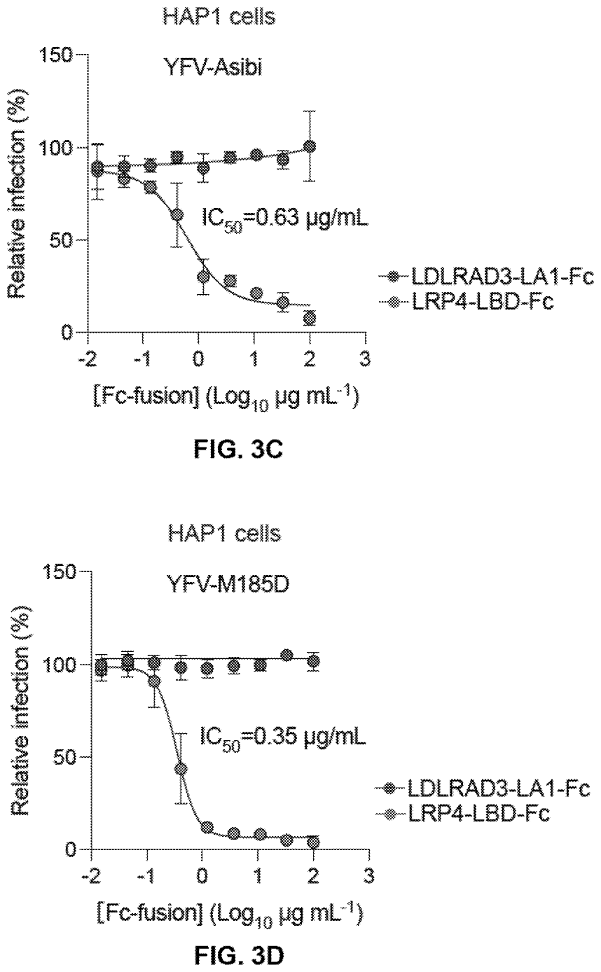

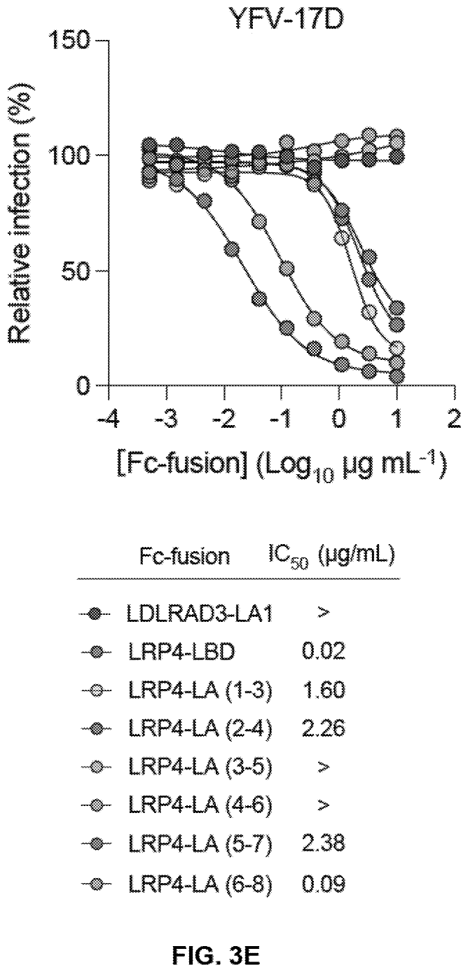

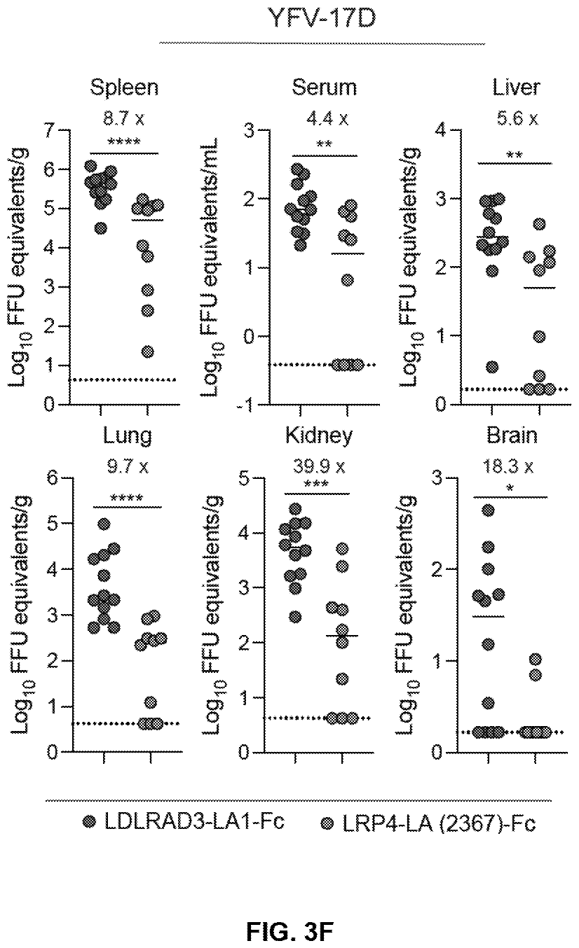

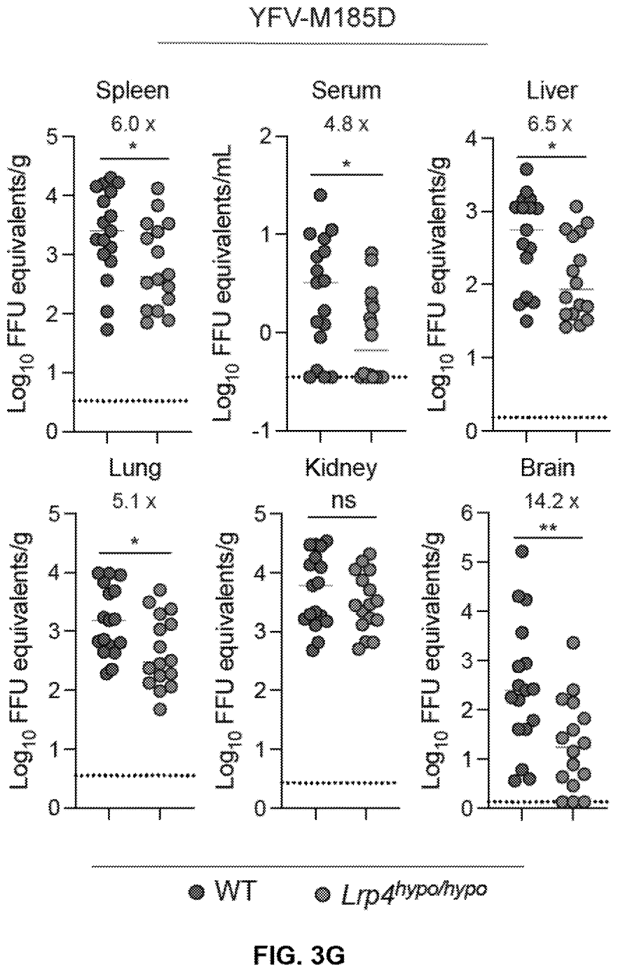

Next, it was assessed whether LRP4-LBD-Fc proteins could inhibit YFV infection. Pre-incubation of LRP4-LBD-Fc but not LDLRAD3-LA1-Fc with YFV-17D dose-dependently reduced infection in HAP1, HEK293T and Vero cells (FIG. 3A, FIG. 8E, FIG. 8F). As YFV targets the liver in humans, it was confirmed that LRP4-LBD-Fc protein inhibited YFV-17D infection in human HepG2 cells of hepatic origin (FIG. 3B). Infection by YFV-Asibi and YFV-M185D viruses in HAP1 cells also was blocked by LRP4-LBD-Fc protein (FIG. 3C, FIG. 3D). To determine which of the 8 LA domain(s) (LA1-8) is important for interacting with YFV during infection, LRP4 proteins composed of different LA domains were produced and performed neutralization assays. Pre-incubation of YFV-17D with Fc fusion proteins containing single LA domains did not block infection in HAP1 cells (FIG. 8G); consistent with these results, expression of single LRP4 LA domains did not support YFV-17D infection in K562 cells (FIG. 8I, FIG. 8L). Most two-LA-domain Fc fusion proteins also did not neutralize YFV-17D infection, with the exception LRP4-LA(2-3)-Fc and LRP4-LA(6-7)-Fc proteins, which inhibited infection, albeit less efficiently than LRP4-LBD-Fc (FIG. 8H). However, multiple three-LA-domain-containing Fc fusion proteins neutralized YFV-17D infection in HAP1 cells, including LRP4-LA(1-3)-Fc, LRP4-LA(2-4)-Fc, LRP4-LA(5-7)-Fc and LRP4-LA(6-8)-Fc proteins. Among these proteins, LRP4-LA(6-8)-Fc protein blocked YFV infection almost as well as the full-length LRP4-LBD-Fc (half-maximum inhibitory concentration (IC₅₀) values of 0.09 and 0.02 μg ml-1, respectively; FIG. 3E). Similarly, the corresponding LA domains promoted YFV-17D infection in K562 cells (FIG. 8J, FIG. 8K, FIG. 8L). These results suggest that engagement of multiple LA domains may be required for avid LRP4 binding to YFV.

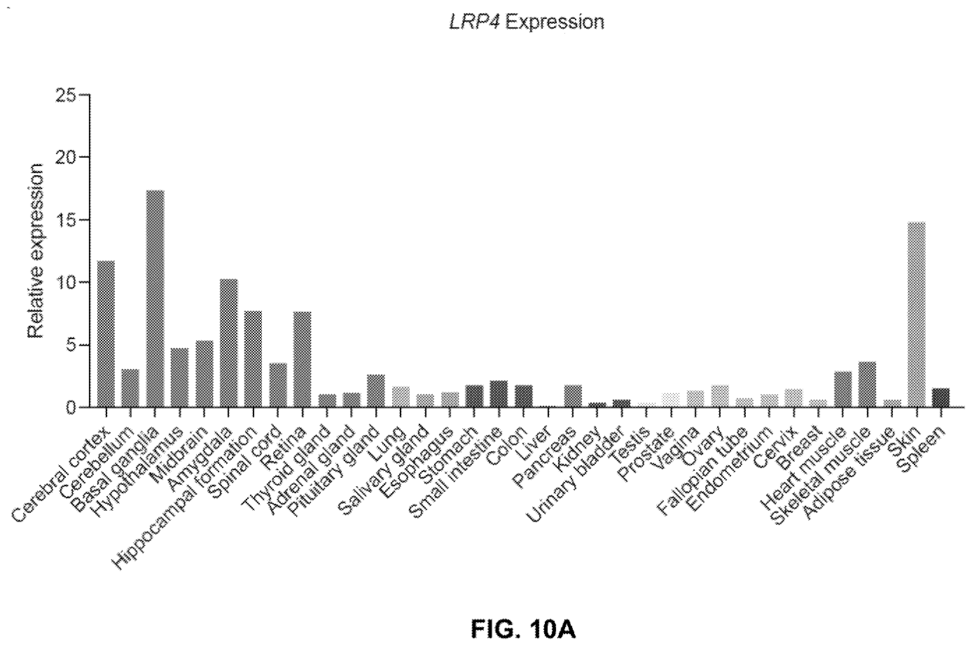

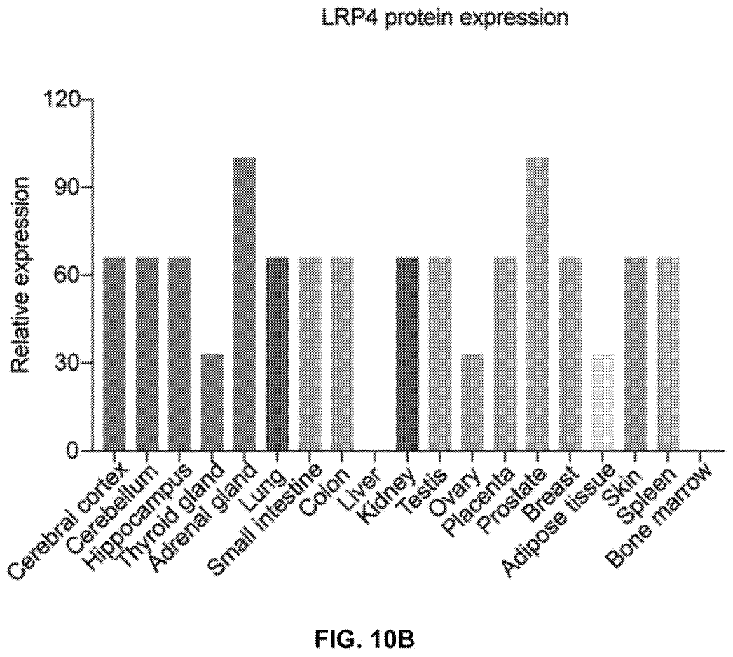

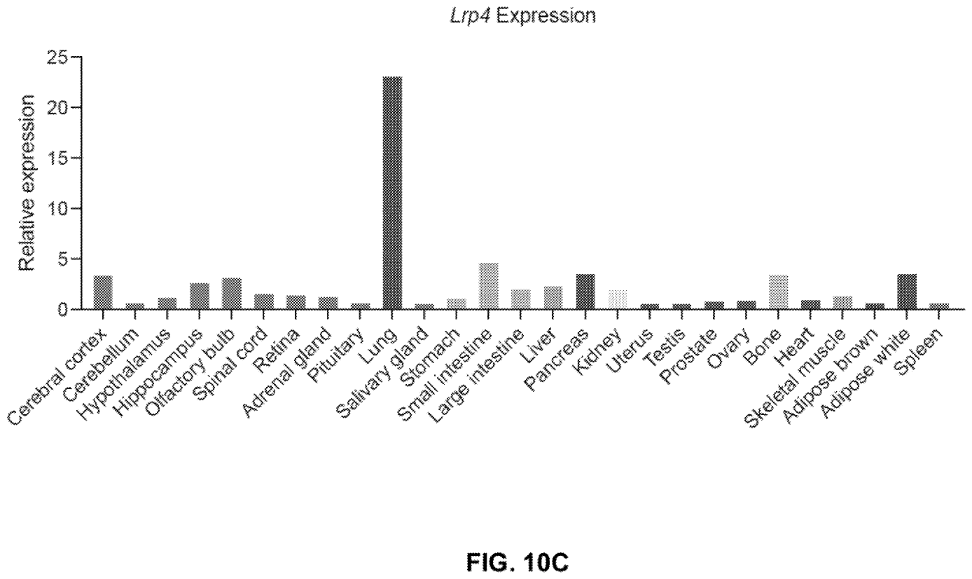

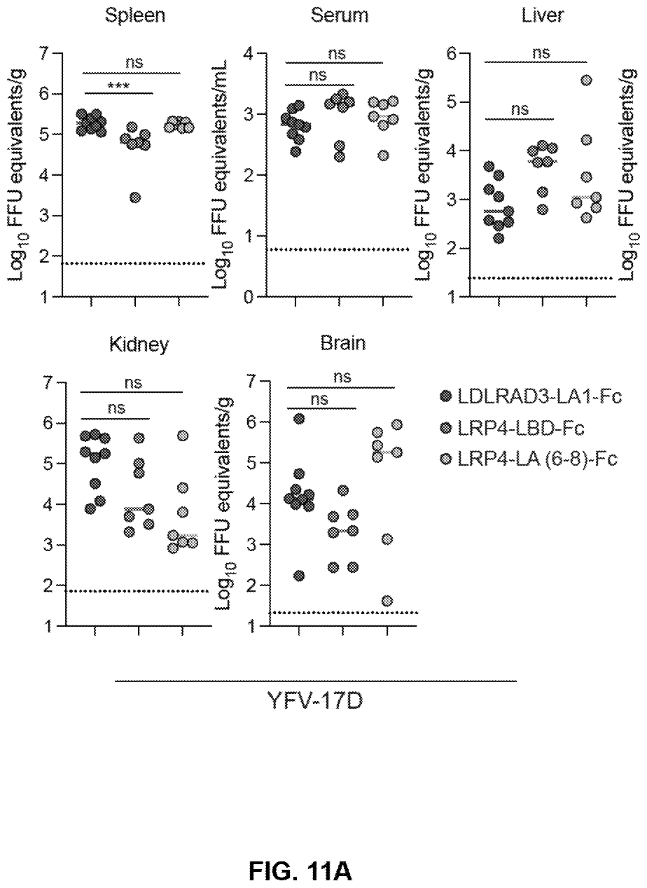

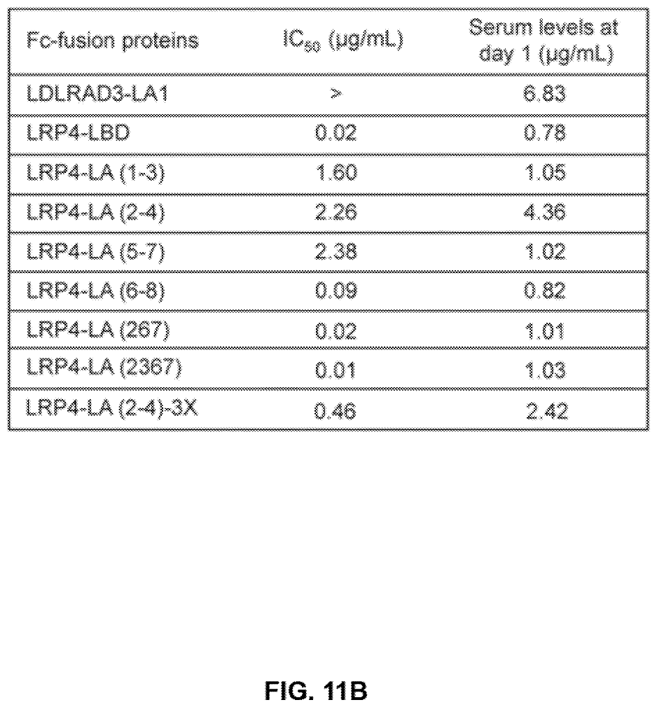

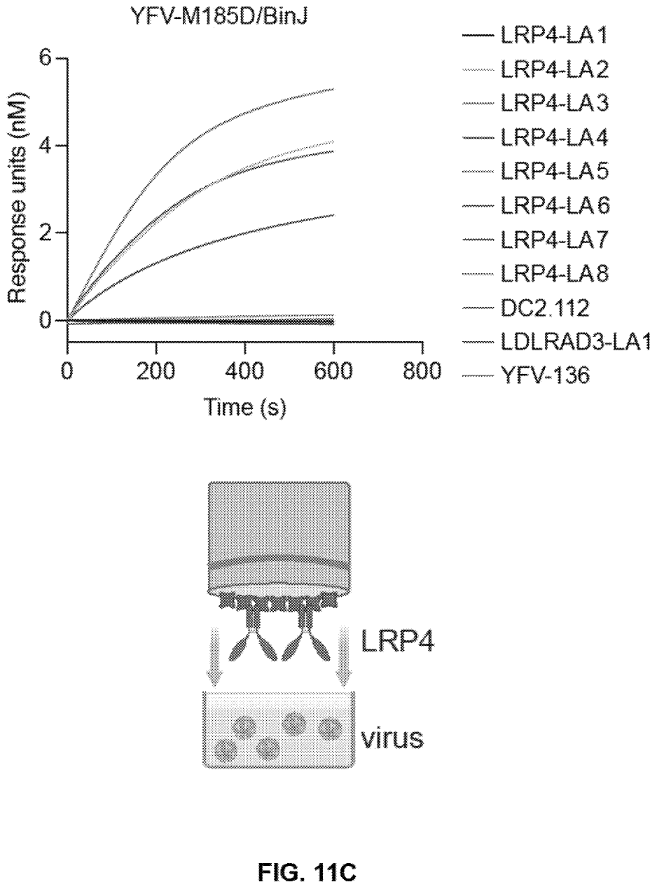

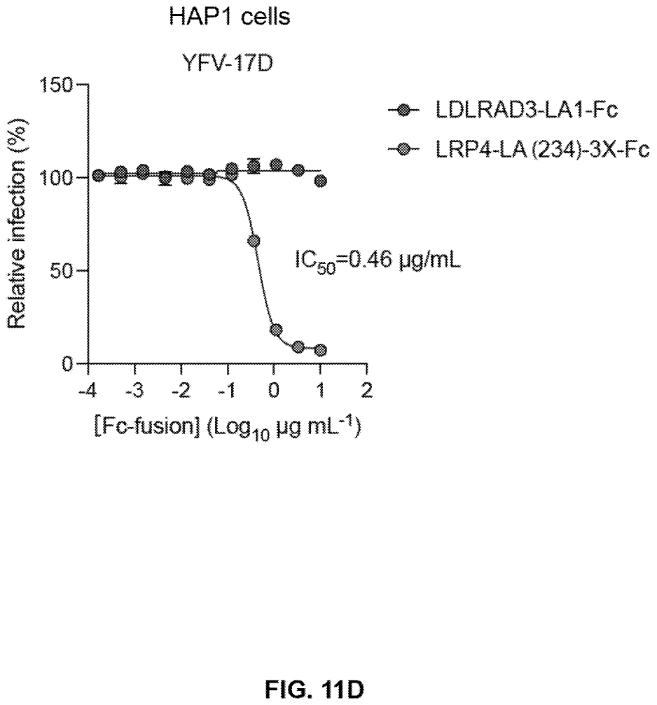

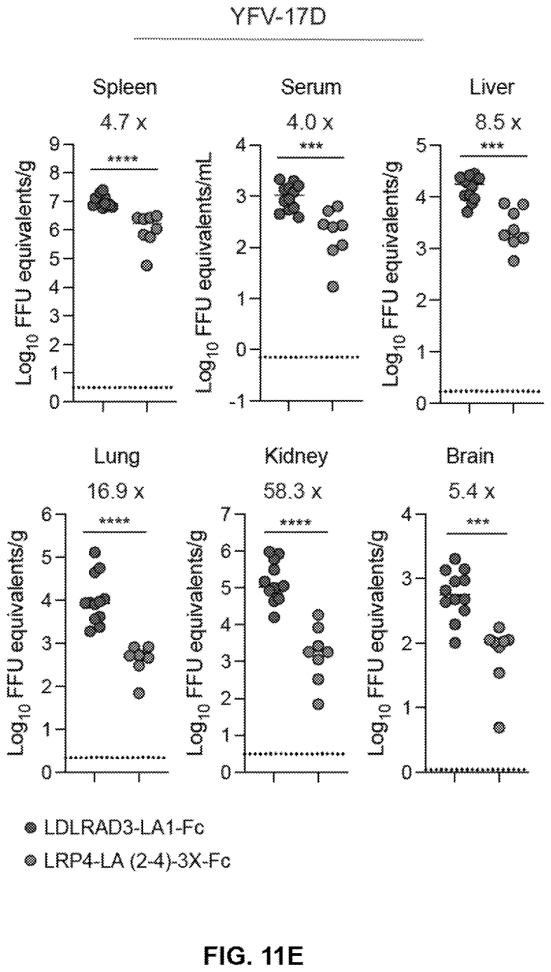

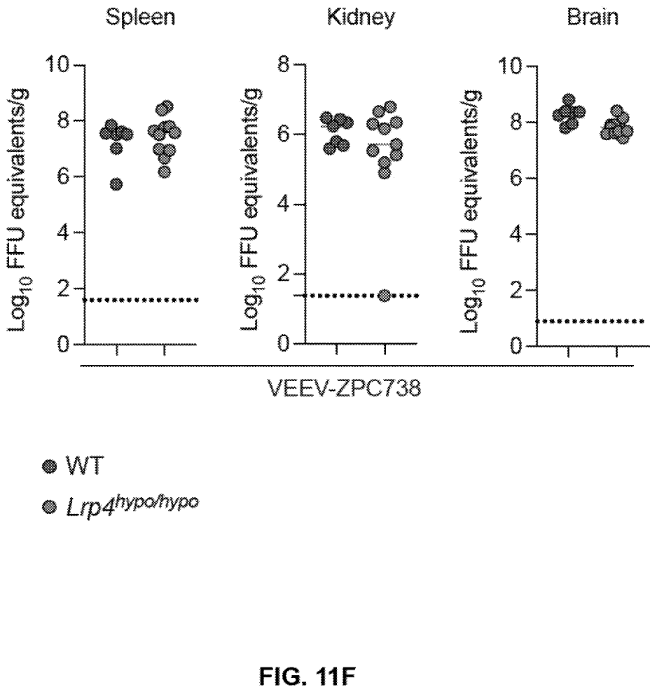

To begin to explore a physiological role of LRP4 in YFV infection, its expression pattern was assessed using deposited RNA sequencing (RNA-seq) and proteomics data. Human LRP4 was more highly expressed in the brain and skin, whereas the highest levels of mouse Lrp4 were present in the lung (FIG. 10A, FIG. 10B, FIG. 10C); however, LRP4 and Lrp4 mRNA or protein were expressed at lower levels in the liver, a key target of YFV infection. Next, it was evaluated whether LRP4-Fc decoy proteins could block YFV infection in mice. A caveat of mouse models of YFV is that hepatocytes generally are resistant to infection, even in immunocompromised mice. This study used 5- to 6-week-old AG129 mice, which lack both interferon-α (IFNα) and IFNγ receptors, as these animals support high levels of YFV infection in the bloodstream and central nervous system. First, mice were administered an LRP4-LBD-Fc decoy (500 μg; approximately 20 mg per kg) 4 h before YFV-17D infection. However, compared to LDLRAD3-LA1-Fc protein, LRP4-LBD-Fc did not inhibit YFV infection (FIG. 11A), and similar results were observed with an LRP4-LA(6-8)-Fc decoy (FIG. 11A). As this result was unexpected, serum levels of the decoy molecules were measured. In comparison to the LDLRA3-LA1-Fc decoy molecule, which inhibits VEEV infection in mice, the levels of LRP4-LBD-Fc and LRP4-LA(6-8)-Fc in the serum were lower (FIG. 11B), suggesting poor bioavailability. To overcome this limitation, additional BLI assays were performed to determine which single LRP4 LA domains could engage YFV virions; these experiments showed that YFV virions can bind to LA2, LA6 or LA7 domains when the protein is placed in the solid phase and multivalent interactions can occur (FIG. 11C). On the basis of these data, additional domain truncated LRP4-based decoy molecules were generated and assessed neutralizing activity; for those proteins with substantial inhibitory activity, serum levels were measured after administration to naive mice. Among these, LRP4-LA(2-4)-Fc showed the highest levels in the serum (FIG. 11B). Next, an LRP4-LA(2-4)-3×-Fc decoy protein was generated, which contains three LA(2-4) repeats and shows increased neutralizing activity (FIG. 11D). Administration of LRP4-LA(2-4)-3×-Fc protein resulted in reduced YFV-17D viral RNA levels in the spleen, serum, liver, lung, kidney and brain tissues of AG129 mice compared with LDLRAD3-LA1-Fc protein (FIG. 11E). Also tested was an LRP4-LA(2,3,6,7)-Fc decoy, which showed the highest inhibitory activity against YFV-17D in cell culture and slightly improved serum levels in C57BL/6J mice. To facilitate infection in C57BL/6J mice, animals were pretreated with an anti-IFNAR1 antibody (MAR1-5A3) to block type I IFN signaling. Mice administered with LRP4-LA(2,3,6,7)-Fc protein sustained lower viral RNA levels in the spleen, serum, liver, lung, kidney and brain tissues compared with mice treated with LDLRAD3-LA1-Fc protein (FIG. 3F). To extend these findings, YFV infection was assessed in commercially available Lrp4^{tm1Her}/J (Lrp4^{hypo/hypo}) mice, which encode a truncated LRP4 protein; in these mice with hypomorphic alleles, LRP4 expression is diminished but not absent, as a complete gene knockout is lethal at the embryonic stage. Littermate wild-type and Lrp 4^{hypo/hypo} mice were treated with anti-IFNAR1 antibody and inoculated them with the pathogenic YFV-M185D strain. At 5 days post-infection (d.p.i.), moderately lower viral RNA levels were observed in the spleen, serum, liver, lung and brain tissues (2.9-65-fold, P<0.05) in Lrp4^{hypo/hypo} mice compared with in wild-type mice (FIG. 3G). By contrast, differences in infection by VEEV, an unrelated alphavirus, in wild-type and Lrp4^{hypo/hypo} mice were not apparent (FIG. 11F).

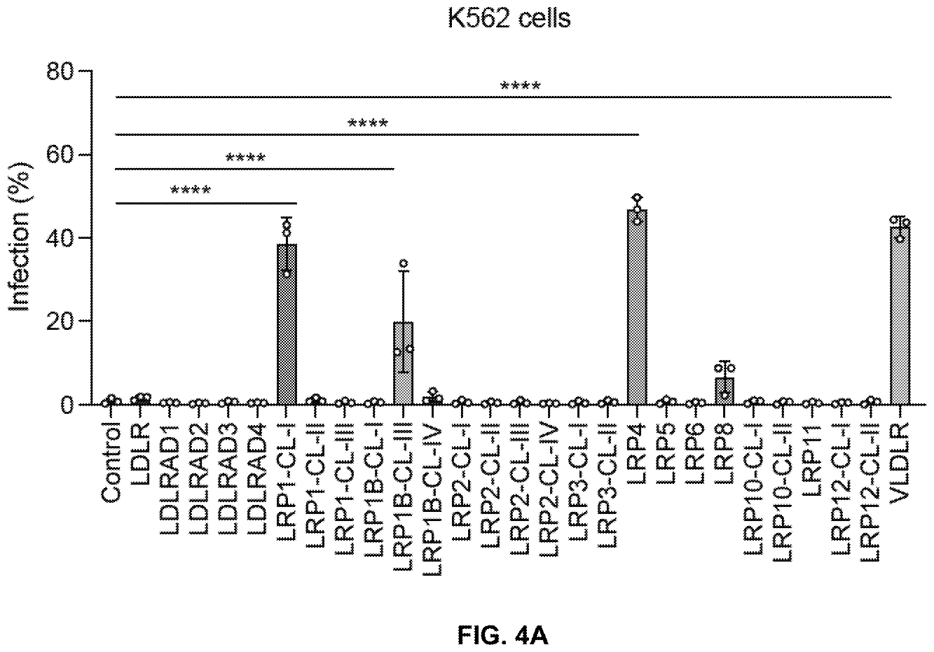

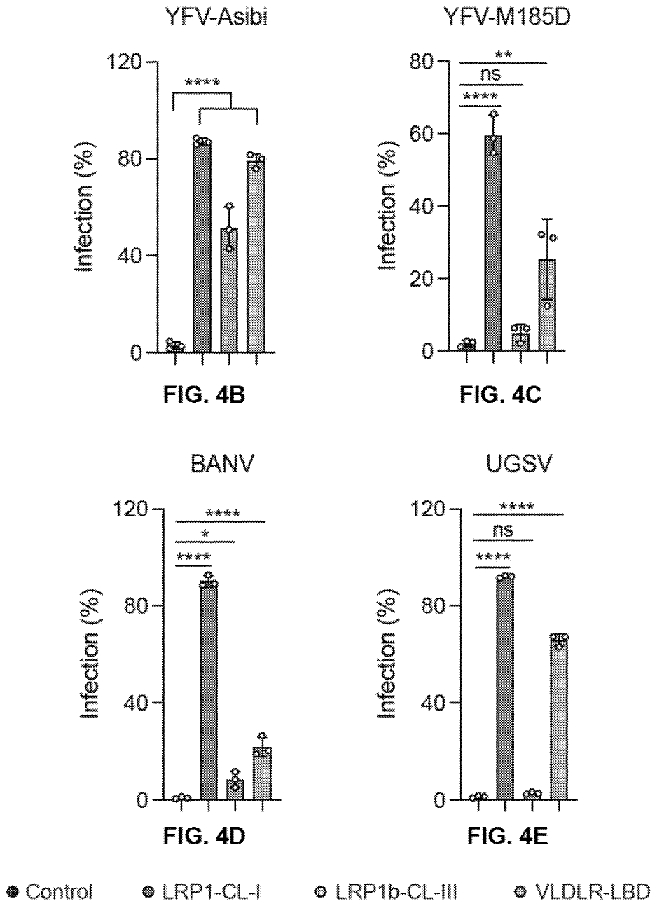

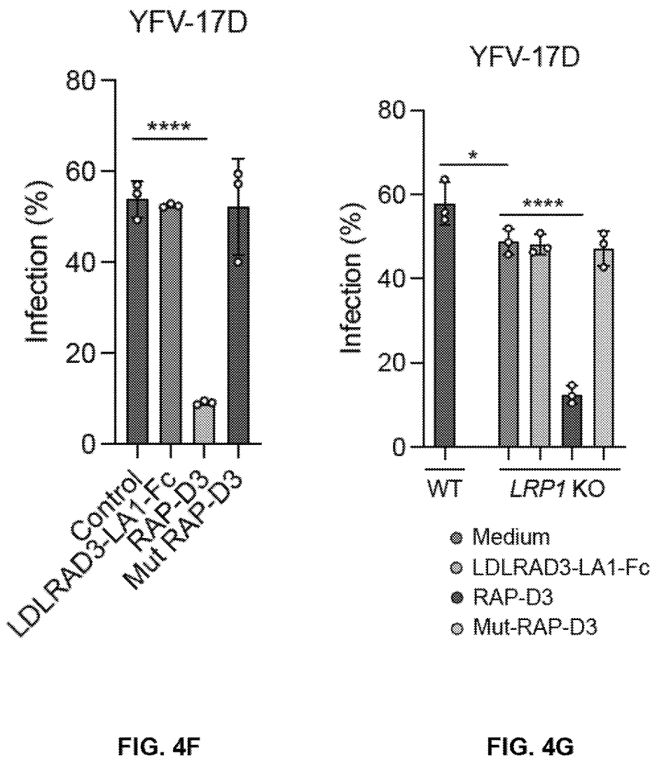

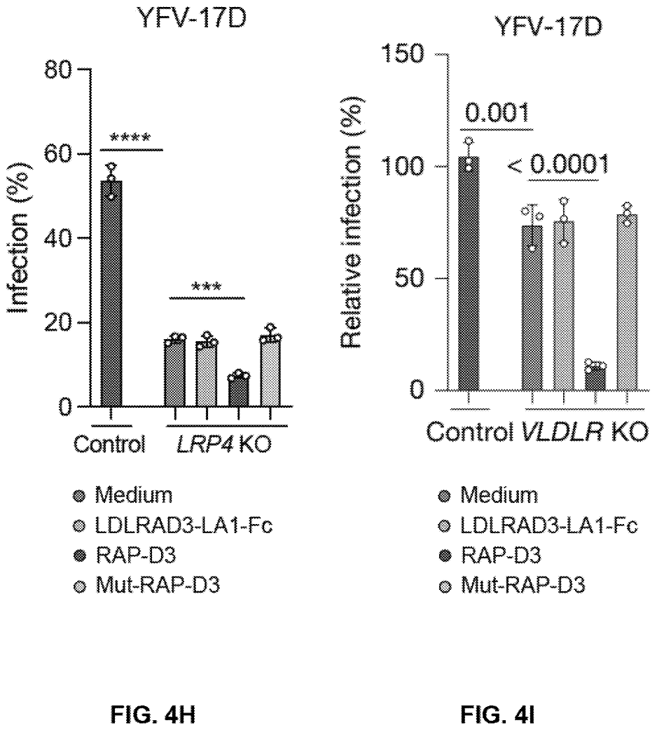

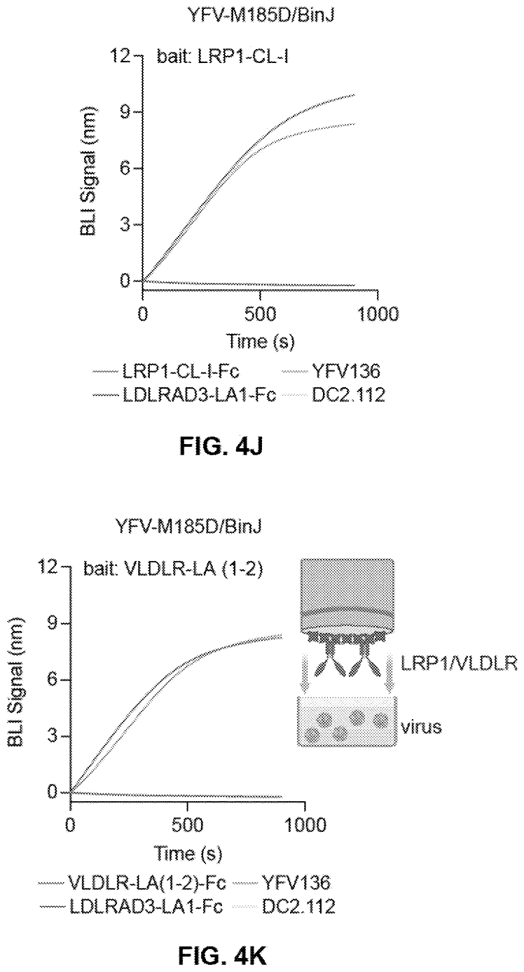

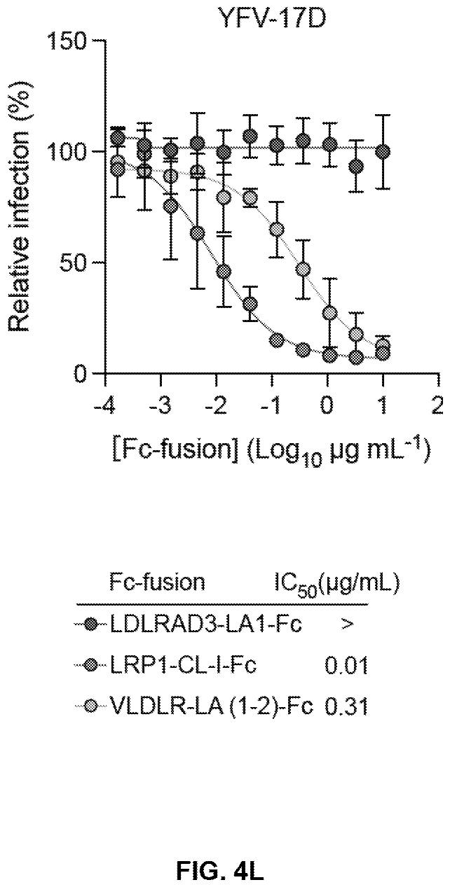

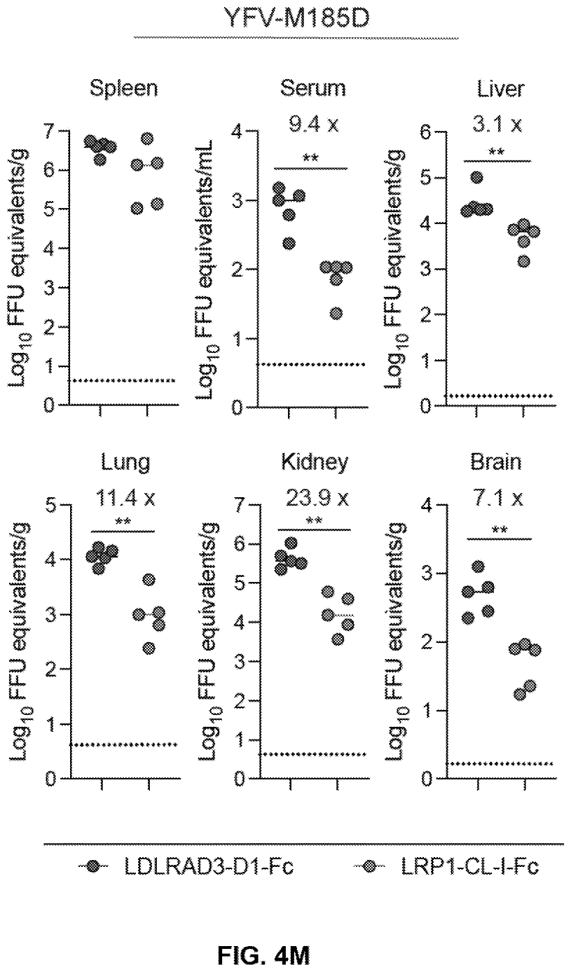

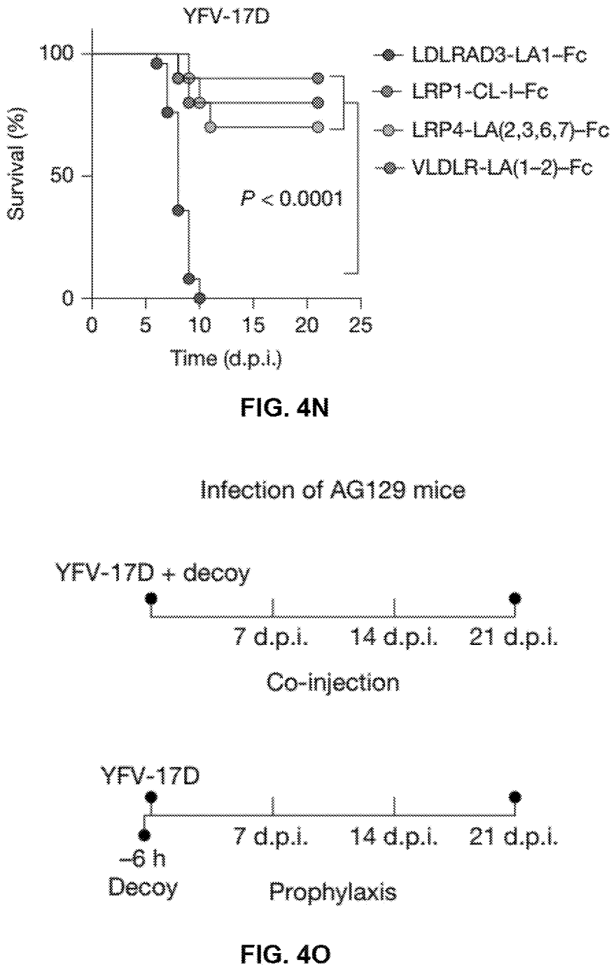

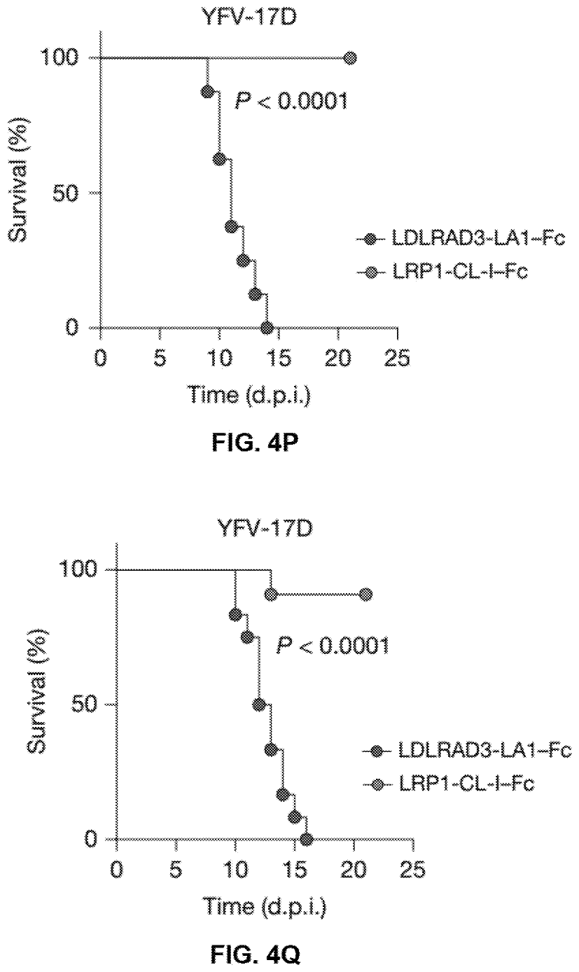

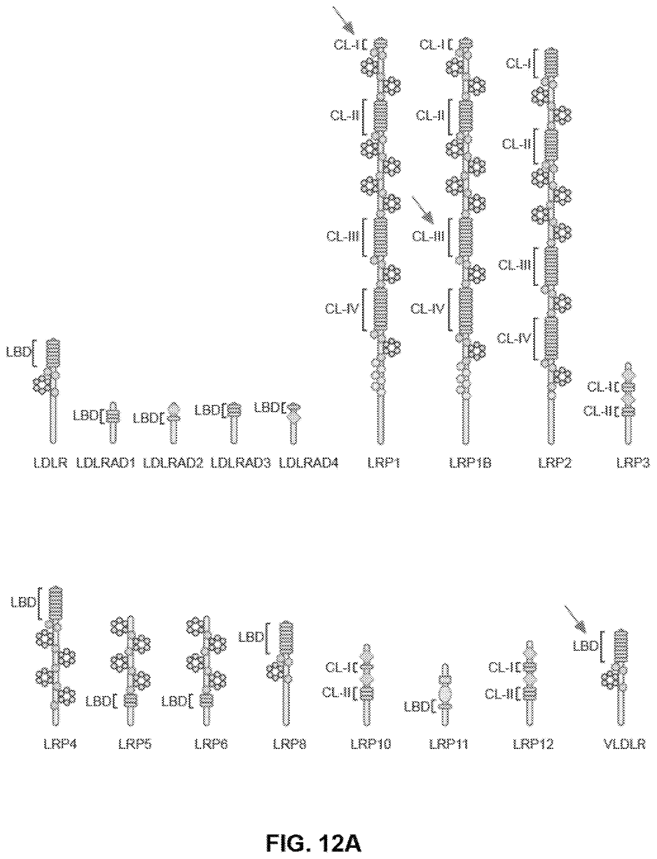

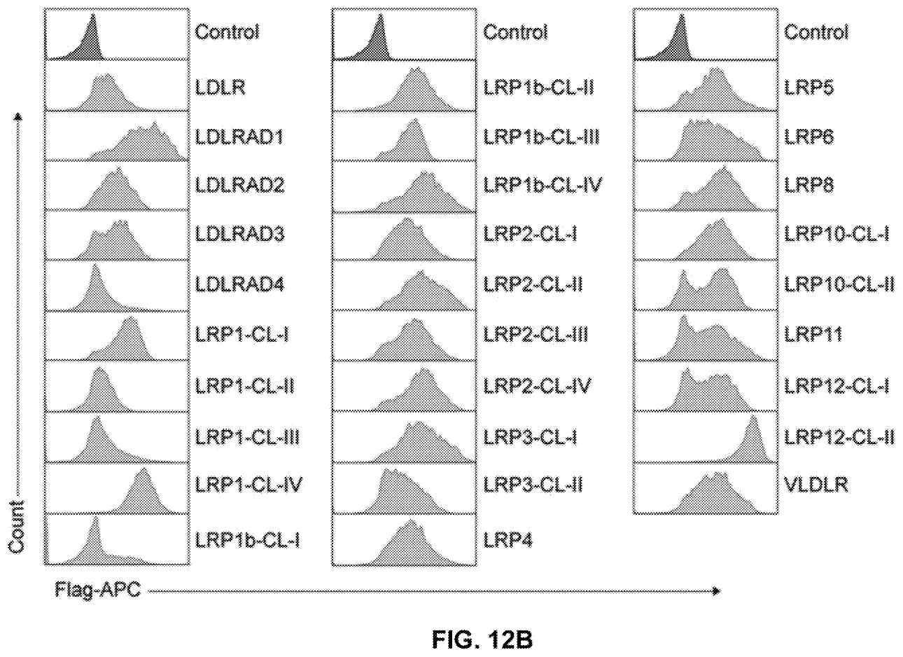

As residual YFV infection was evident in ΔLRP4 cells and tissues of Lrp4^{hypo/hypo} mice, additional entry receptors might exist. As recent studies suggested that other LDLR family members might act as entry factors for JEV and WNV (LDLR) or DENV (LRP1), the LBD of 17 different LDLR family members was ectopically expressed in K562 cells and tested them for YFV infection. As some of the LDLR family member proteins (such as LRP1, LRP1B, LRP2, LRP3, LRP10 and LRP12) are too large for the lentivirus vector, their LBDs were divided for expression (FIG. 12A). Using this approach, it was observed that LRP1 cluster I (LRP1-CL-I (LA1-2)), LRP1B-CL-III (LA11-20) and VLDLR also could support infection of YFV-17D RVPs in K562 cells (FIG. 4A, FIG. 12B). Next, it was tested whether these proteins promoted infection of two other strains of YFV or other members of the YFV antigenic complex. Ectopic expression of LRP1-CL-I in K562 cells enhanced infection of YFV-Asibi, YFV-M185D, BANV and UGSV (FIG. 4B, FIG. 4C, FIG. 4D, FIG. 4E). VLDLR also promoted infection of YFV and related viruses, albeit with variation in magnitude. By contrast, expression of LRP1B-CL-III robustly enhanced infection only of YFV-Asibi virus, but not YFV-M185D, BANV or UGSV (FIG. 4B, FIG. 4C, FIG. 4D, FIG. 4E).

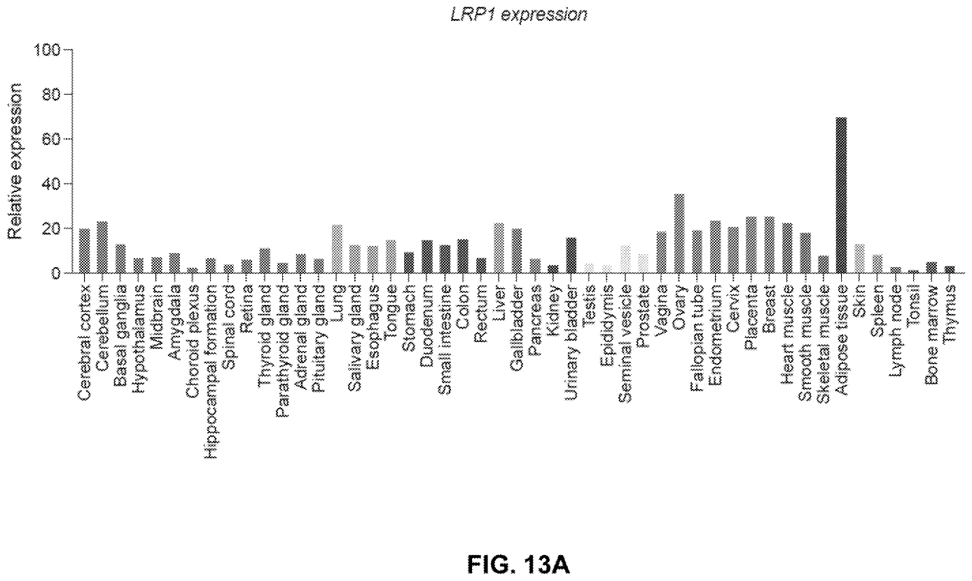

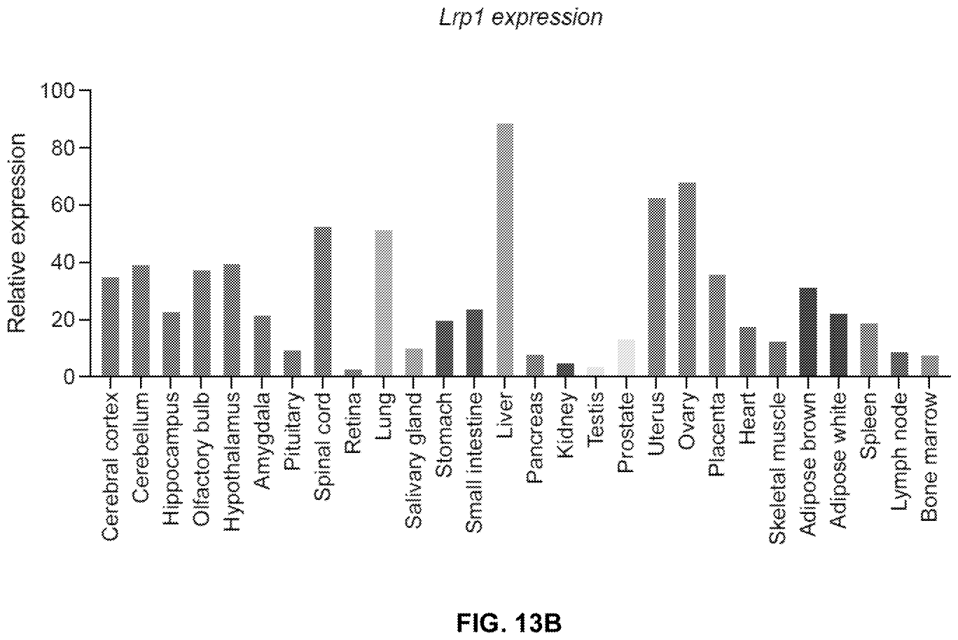

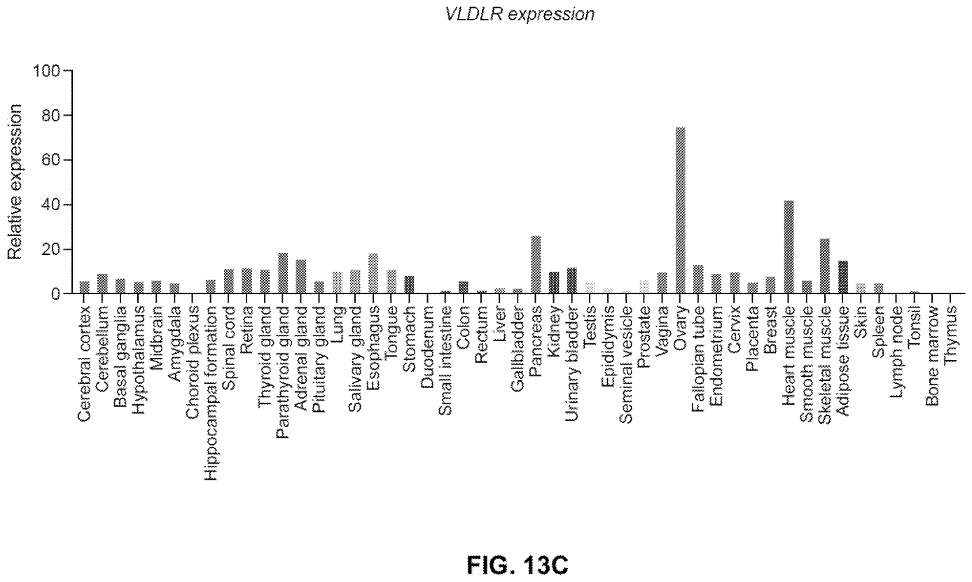

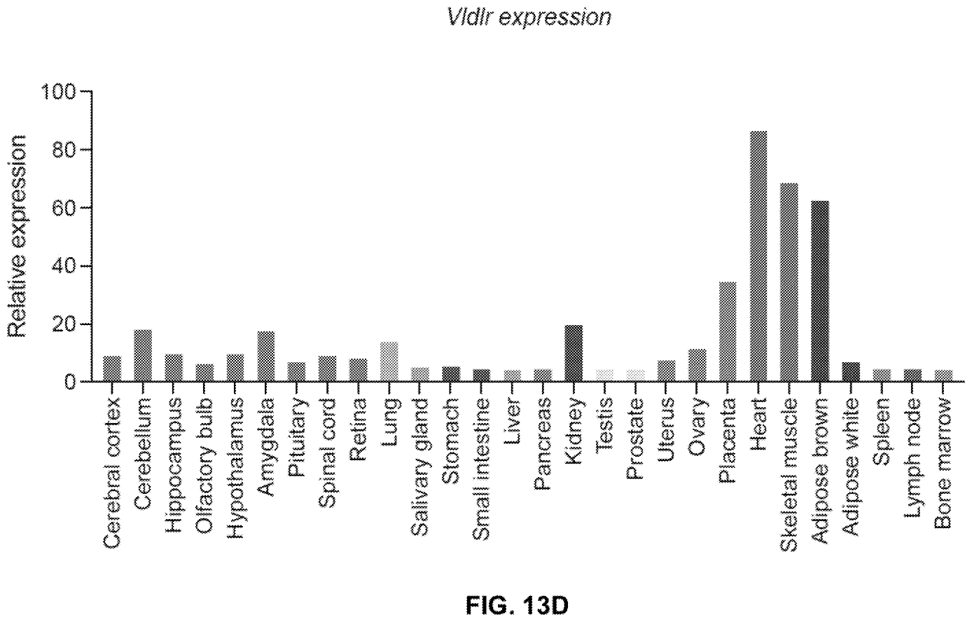

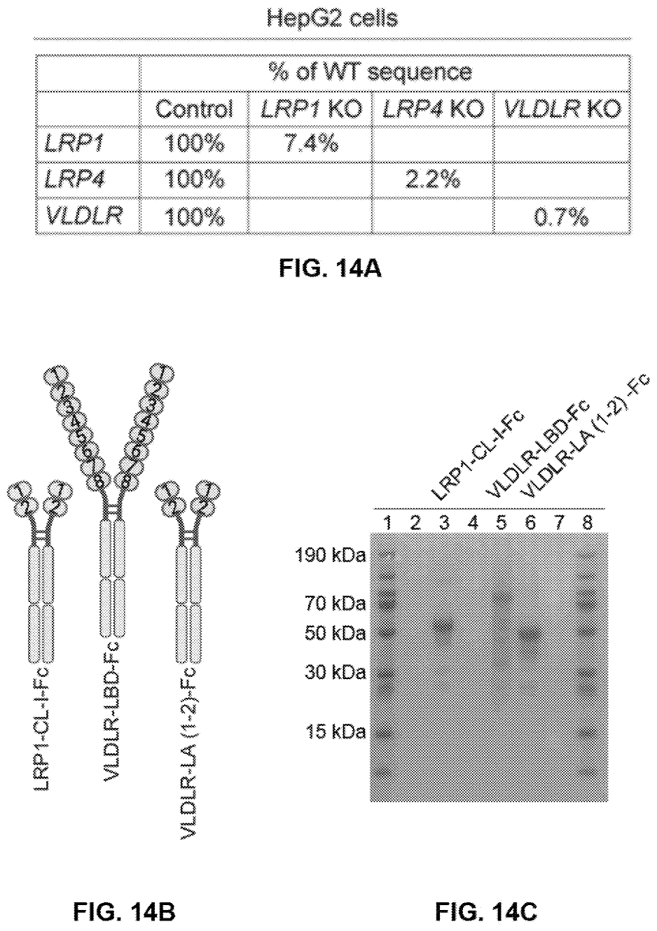

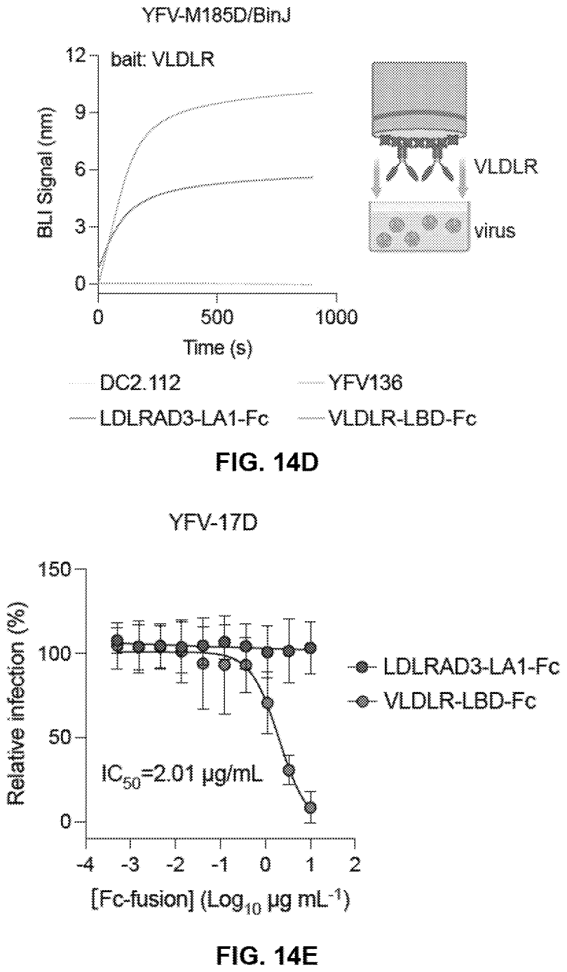

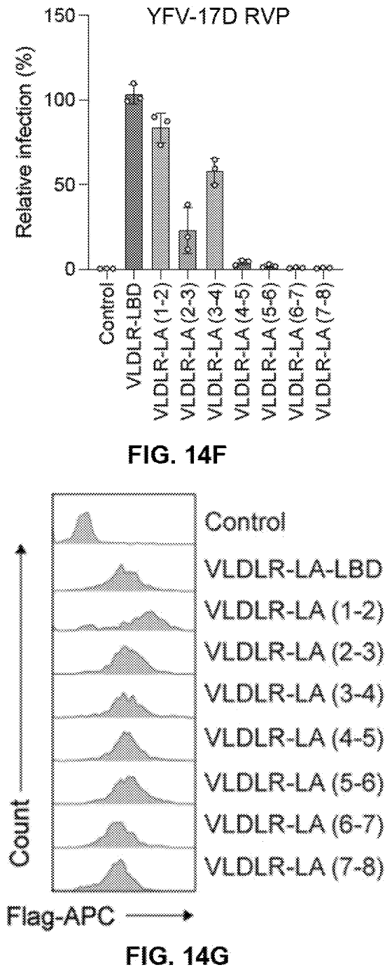

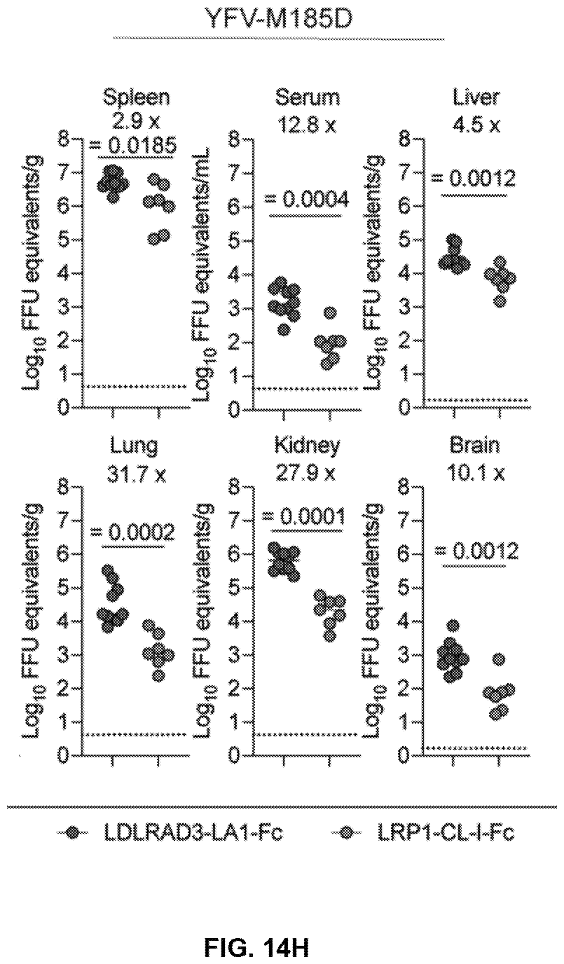

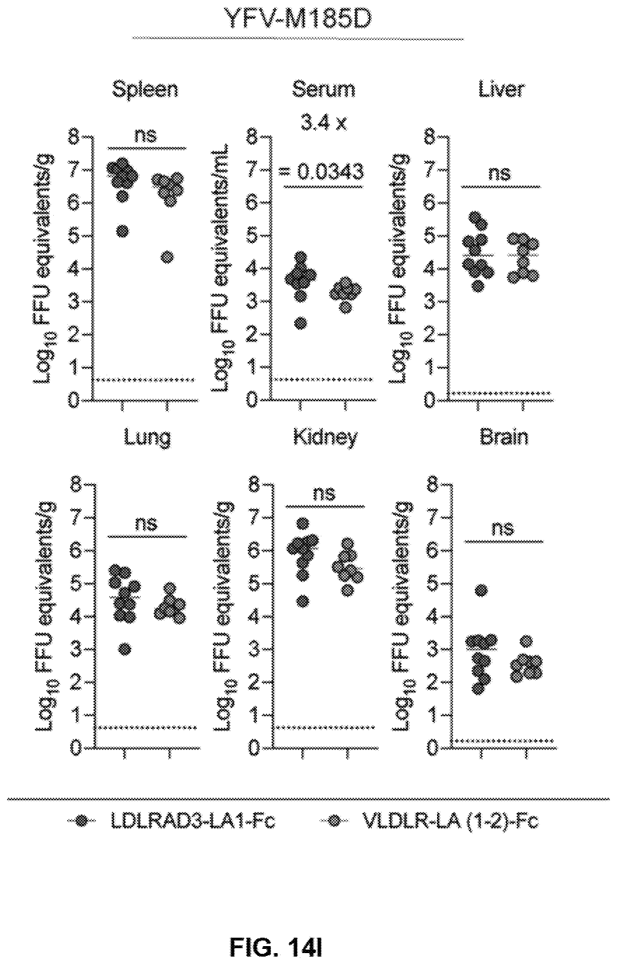

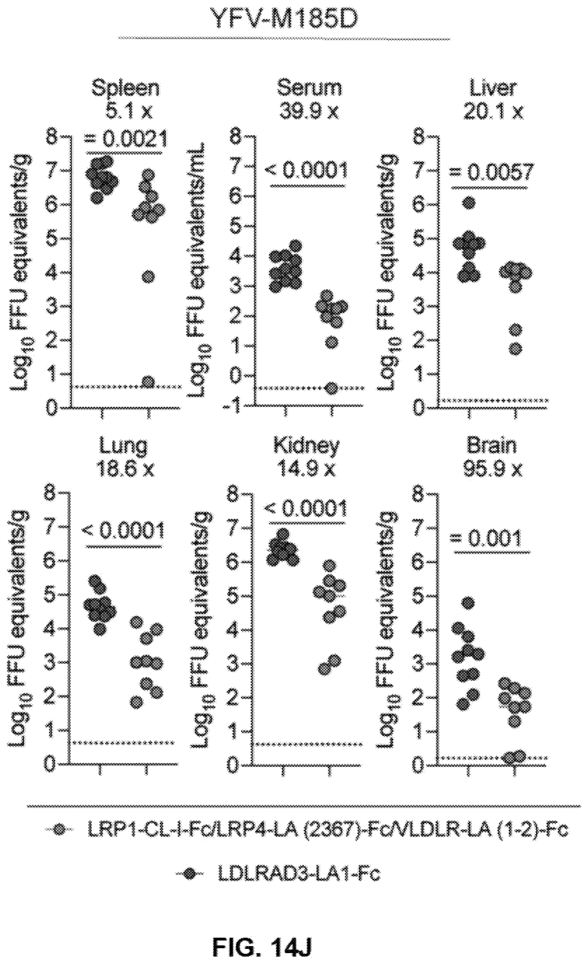

On the basis of these results, the role of LRP1 and VLDLR in YFV infection was explored. Published RNA-seq data showed differences in expression patterns of LRP1 and VLDLR: LRP1, but not VLDLR, is expressed in the liver, a principal target tissue for YFV infection (FIG. 13A, FIG. 13B, FIG. 13C, FIG. 13D). Accordingly, it was evaluated whether blocking of LRP1 and VLDLR affected YFV infection. Receptor-associated protein (RAP) is a chaperone that binds to many LDLR-related receptors; soluble forms of RAP can block LRP1 and VLDLR engagement with endogenous or viral ligands. Pre-incubation of HepG2 cells with domain 3 of mouse RAP (mRAP-D3) inhibited YFV-17D virus infection, whereas an mRAP-D3 mutant with two lysine substitutions that abrogate LA domain binding (K256A/K270E) did not (FIG. 4F, FIG. 4G, FIG. 4H, FIG. 4I). Consistent with these results, editing of LRP1, LRP4 or VLDLR expression in HepG2 cells each partially reduced YFV-17D infection (FIG. 4G, FIG. 4H, FIG. 4I, FIG. 14A).

To evaluate whether LRP1 and VLDLR directly interact with YFV, binding and neutralization assays were performed. LRP1-CL-1-Fc and VLDLR-LBD-Fc fusion proteins bound to YFV virions (FIG. 4J, FIG. 4K, FIG. 14B, FIG. 14C, FIG. 14D) and inhibited YFV-17D infection in HepG2 cells (FIG. 4L, FIG. 14E). While LRP1-CL-I has only two LA domain repeats, VLDLR LBD has eight LA domain repeats. To evaluate which VLDLR LA domains could optimally support YFV infection, tandem LA domains of VLDLR was expressed in K562 cells. Expression of VLDLR-LA(1-2) conferred similar levels of YFV infection compared with VLDLR-LBD (FIG. 14F, FIG. 14G). Moreover, VLDLR-LA(1-2)-Fc bound to and dose-dependently inhibited YFV infection in cell culture (FIG. 4K, FIG. 4L, FIG. 14B, FIG. 14C).

To assess a functional role of LRP1 and VLDLR in vivo, this study administered LRP1-CL-I-Fc or VLDLR-LA(1-2)-Fc decoy molecule to anti-IFNAR1-antibody-treated C57BL/6J mice before or concurrent with YFV-M185D infection. While viral RNA levels were reduced in all of the samples tested of mice treated with LRP1-CL-I-Fc decoy compared with LDLRAD3-LA1-Fc (FIG. 4M, FIG. 14H), this phenotype was not observed in mice treated with VLDLR-LA(1-2)-Fc, except for a small reduction in the serum (FIG. 14I). When all three decoy proteins (LRP4-LA(2,3,6,7)-Fc, LRP1-CL-I-Fc and VLDLR-LA(1-2)-Fc) were concurrently administered, YFV RNA levels were greatly reduced in multiple tissues compared with in mice treated with LDLRAD3-LA1-Fc protein (FIG. 14J).

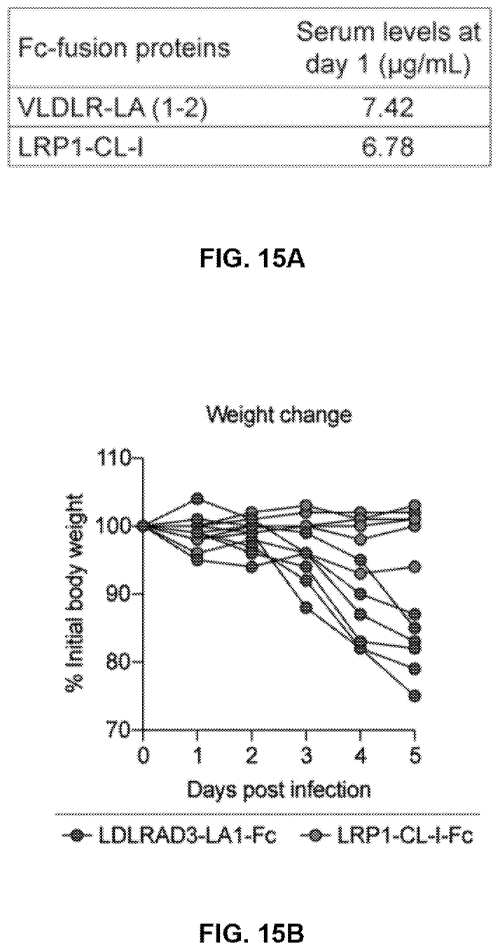

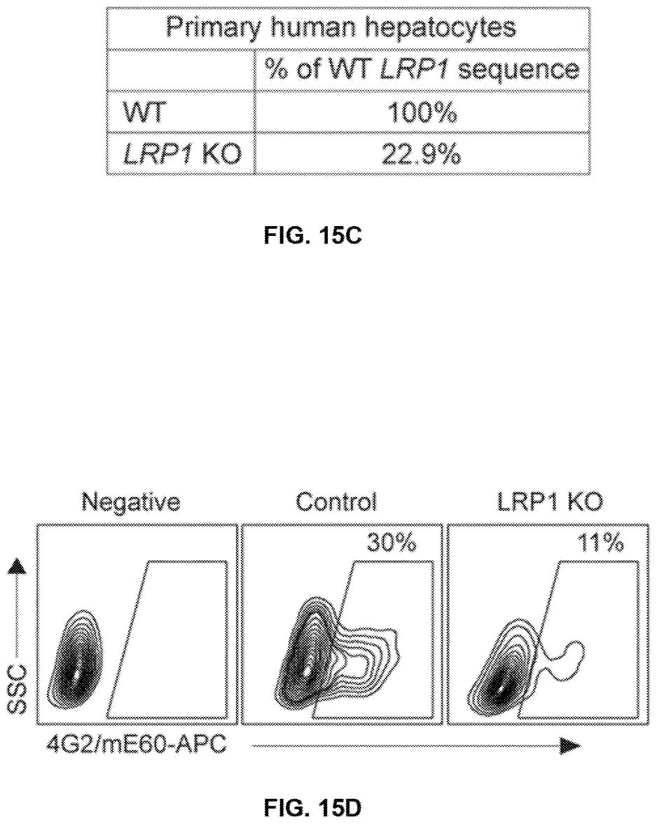

Next, it was investigated whether LRP1-Fc, LRP4-Fc and VLDLR-Fc decoys can protect mice from lethal YFV infection. C57BL/6J mice were treated with anti-IFNAR1 antibody and inoculated intracranially with 10² plaque-forming units (PFU) of YFV-17D. Pre-incubation of YFV-17D with LRP4-LA(2,3,6,7)-Fc, LRP1-CL-I-Fc or VLDLR-LA(1-2)-Fc improved survival compared with in mice receiving the LDLRAD3-LA1-Fc decoy (FIG. 4N). To extend these results to peripheral YFV challenge, the LRP1-CL-I-Fc decoy was used due to its favorable neutralizing activity and serum level profiles (FIG. 4L, FIG. 15A). Pre-incubation of virus or prophylaxis of AG129 mice with LRP1-CL-I-Fc protected from YFV-induced mortality (FIG. 4O, FIG. 4P, FIG. 4Q).

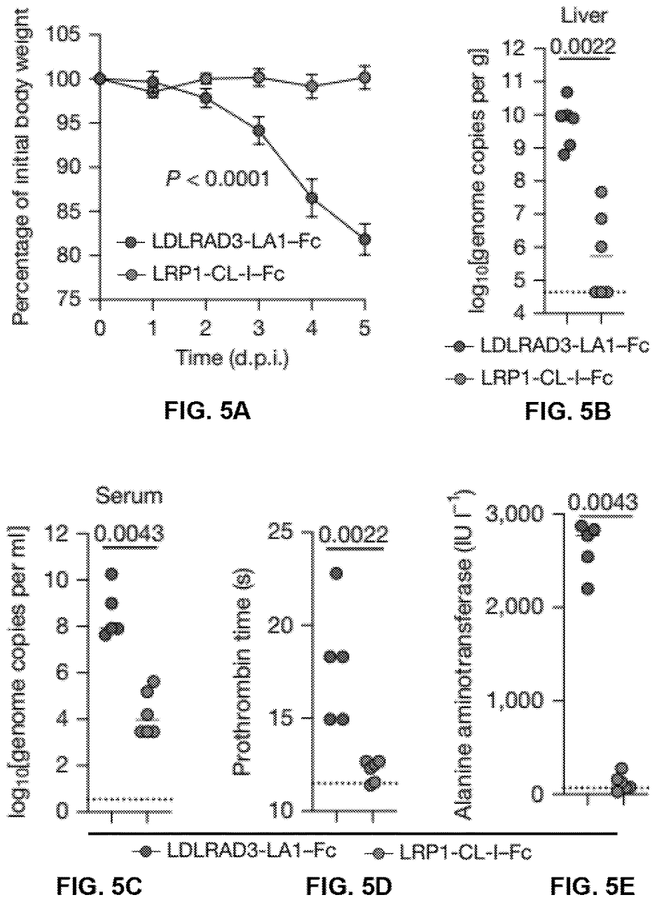

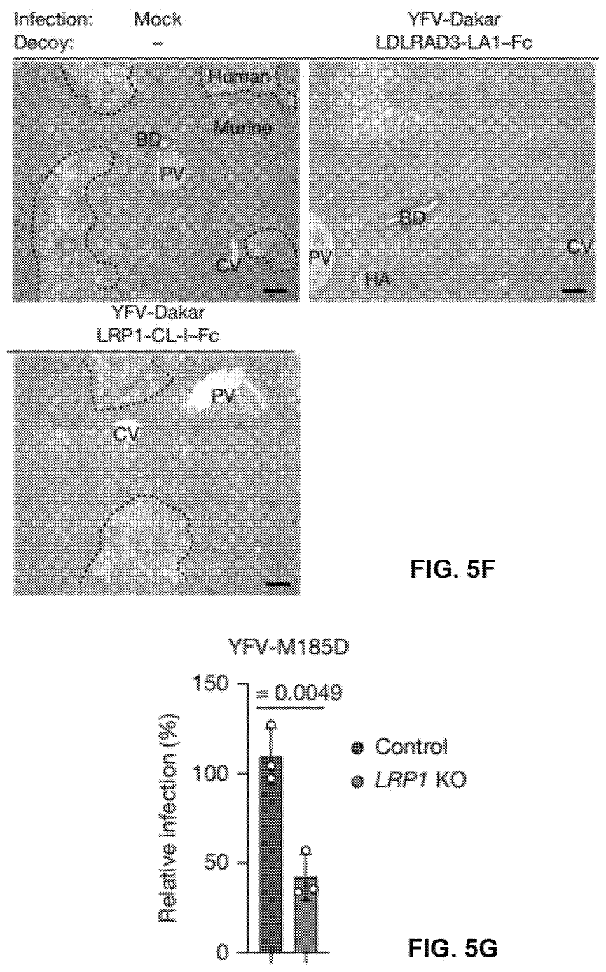

To examine the use of LRP1 in an animal model that displays a viscerotropic pattern of YFV infection, studies were performed in Fah^{-/-} (encoding fumarylacetoacetate hydrolase), Rag2^{-/-} and Il2rg^{-/-} triple-knockout mice (hereafter FRG mice) engrafted with human hepatocytes (hFRG), which sustain massive hepatic infection and injury by wild-type YFV strains. hFRG mice were treated with LRP1-CL-I-Fc or LDLRAD3-LA1-Fc decoy at 6 h before and 24 h after YFV-Dakar infection. At 5 d.p.i., hFRG mice administered LRP1_CL-I-Fc had reduced YFV viral loads in liver and serum and were protected from weight loss, coagulopathy (prothrombin time (PT)) and liver injury (serum alanine transaminase (ALT) and pathology) (FIG. 5A, FIG. 5B, FIG. 5C, FIG. 5D, FIG. 5E, FIG. 5F, FIG. 15B). Finally, because LRP1 is more highly expressed in human liver tissue than LRP4 or VLDLR (FIG. 10A, FIG. 13A, FIG. 13C), LRP1-deficient primary human hepatocytes (PHHs) were generated. Gene editing of LRP1 reduced YFV infection compared with in the control PHHs (FIG. 5G, FIG. 15C, FIG. 15D).

### DISCUSSION

The CRISPR screen identified LRP4 as an entry receptor that promotes infection of YFV and other related viruses in the YF antigenic complex. Infection studies in ΔLRP4 HAP1 cells and Lrp4^{hypo/hypo} mice also indicated that additional host factors probably contribute to YFV entry. Indeed, subsequent screening of LDLR family members suggested that LRP1, VLDLR and possibly LPR1B can promote YFV infection. The use of multiple LDLR family members as receptors for a given virus is reminiscent of a paradigm established for the distantly related alphaviruses. Moreover, LRP1 was recently characterized as a receptor for several bunyaviruses, including Oropouche and Rift Valley fever viruses, and has been implicated in DENV infection. The differential expression of LDLR family receptors in target tissues could in part explain YFV tropism. In human mRNA-seq datasets, LRP4 and VLDLR are expressed at low levels in hepatocytes, whereas LRP1 is expressed at higher levels. Indeed, loss of LRP1 expression in PHH reduced YFV infection. On the basis of these results and the soluble decoy experiments in vivo, LRP1 may be a dominant receptor on hepatocytes for YFV. LRP4 and VLDLR may have roles in YFV infection of other permissive cell types.

The analysis of LRP4 orthologues in other mammalian species suggests that the interaction with YFV is evolutionarily conserved. Whether this extends to more distantly related LRP4 orthologues in vector hosts (such as A. aegypti, 38% identical) warrants further study. Although future experiments also are needed to assess how avidly LRP1 and VLDLR orthologues interact with YFV, it was noted that both receptors are highly conserved in mammals (81-100%, depending on the species). Moreover, other factors beyond receptor expression, such as evasion of innate immunity, probably contribute to the species tropism of YFV, as wild-type laboratory mice do not develop severe YFV infection or disease. Breeding of engineered human LRP1, LRP4 or VLDLR transgenic mice with innate-immune-deficient mice could enable the generation of more useful small-animal models of YFV pathogenesis.

The LBD of LRP4 contains eight LA domain repeats. Some viruses appear to have different patterns of LA domain use for entry. Whereas the alphavirus VEEV binds exclusively to the LA1 domain of LDLRAD3, SFV engages one of several single LA domains of VLDLR, and Eastern equine encephalitis virus binds to multiple domains of VLDLR. As the neutralization data showed that no single or tandem LA domain(s) of LRP4 could effectively inhibit virus infection even though YFV virions could bind to single LA (LA2, LA6 or LA7) domains, YFV may need multiple LA domains to engage LRP4 avidly. Indeed, some multidomain LRP4-Fc fusion proteins neutralized YFV infection almost to the level of the LRP4-LBD-Fc protein. The subsequent analysis of LRP1 and VLDLR interactions showed that the first two LA domains of each (LRP1_CL-1 (LA1-2) and VLDLR LA1-2) bound to YFV, supported infection and, when expressed as soluble Fc decoy molecules, neutralized infection.

In contrast to some other described attachment factors of orthoflaviviruses (such as AXL, CD300A or TIM-1), which engage phospholipids on the virion, LRP4 binds directly to YFV E-DIII—a domain speculated to interact with cell surface receptors. Indeed, this result was confirmed using mutagenesis studies. The introduction of opposing charges (Lys/Arg to Glu) at residues 292, 297, 302, 325 or 330 of YFV E-DIII abolished the binding of LRP4-LBD-Fc fusion protein. These residues are highly but not completely conserved among multiple YFV strains and other members of the YFV antigenic complex. The precise structural basis for how YFV engages LRP4, LRP1 or VLDLR, and whether there are additional sites of engagement apart from E-DIII await future study.

Although an LRP4-LBD-Fc decoy blocked YFV infection efficiently in cell culture, it showed little inhibitory effect in vivo. LRP4-LBD-Fc protein had low serum levels in mice, possibly due to its sequestration by endogenous ligands. The generation of LRP4-LA(2-4)-3×-Fc and LRP4-LA(2,3,6,7)-Fc decoy proteins, which had higher serum levels, enabled us to demonstrate inhibitory activity in vivo as a proof of principle-results that were supported by infection experiments in Lrp4^{hypo/hypo} mice. An LRP1_CL-I-Fc decoy protein also inhibited YFV infection in mice, whereas VLDLR-LA(1-2) decoy protein had less activity, possibly due to its lower neutralizing activity. LRP1-Fc, LRP4-Fc and VLDLR-Fc decoy proteins all protected against lethal YFV infection in C57BL/6 mice, and LRP1-Fc prevented YFV-induced hepatic infection and injury in the more relevant hFRG mouse model. The experiments in cell culture and mice suggest that LRP4 and LRP1 might interact more avidly with YFV than does VLDLR. The soluble decoy experiments in mice have inherent limitations, as they do not definitively establish a role for specific receptors in YFV infection in vivo. The generation of animals with complete or tissue-specific knockouts of individual or multiple LDLR family members will be needed to determine their functions in YFV pathogenesis. Nonetheless, the experiments in LRP1-KO PHHs support a role for LRP1 in YFV pathogenesis.

In summary, the study identifies multiple LDLR family members as candidate receptors for YFV and other related viruses. These investigations enhance understanding of the tropism of YFV but also highlight how analogous approaches might be used to define receptors for other orthoflaviviruses. Insights into virus-receptor interactions may allow for the generation of small-molecule, soluble-decoy-receptor or antibody-based countermeasures that inhibit the entry step in infection.

### METHODS

### Cells

HEK293T (ATCC, CRL-3216), Vero (ATCC, CCL-81) and HepG2 (ATCC, HB-8065) cells were propagated in high-glucose Dulbecco's modified Eagle medium (DMEM) supplemented with 10% FBS, GlutaMax, 10 mM HEPES, non-essential amino acids and penicillin-streptomycin. K562 cells (ATCC, CCL-243) and Raji-DC-SIGNR cells were maintained in RPMI-1640 (Thermo Fisher Scientific) supplemented with 10% FBS, GlutaMax, 10 mM HEPES and penicillin-streptomycin. HAP1 (Horizon Discovery, C631) and ΔSLC35B2/XYLT2 HAP1 cells were maintained in Iscove's modified Dulbecco medium (IMDM) supplemented with 10% FBS, GlutaMax, 10 mM HEPES, non-essential amino acids and penicillin-streptomycin. All cell lines were confirmed to be free of mycoplasma contamination.

### Mice

AG129 mice lacking type I and II IFN receptors. Wild-type C57BL/6J (000664) and Lrp4^{tm1Her}/J (013157) mice were purchased from Jackson Laboratories and maintained in a specific pathogen-free facility. The Lrp4^{tm1Her}/J mice were bred as heterozygotes, and littermate Lrp4^{hypo/hypo} and wild-type mice were used in experiments. For all YFV infection experiments, male and female mice aged 5-6 weeks were used. Male and female aged 7-11 weeks were used in VEEV experiments. Experimental procedures were approved by the Washington University School of Medicine Institutional Animal Care and Use Committee (A3381-01) and followed the guidelines of the Guide for the Care and Use of Laboratory Animals.

hFRG mice were generated at Yecuris Corporation. Engrafted mice shipped at 24 weeks of age were maintained on the recommended diet of PicoLab high energy mouse diet with water containing 3.25% dextrose, 80 mg ml⁻¹ of sulfamethoxazole, 16 mg ml⁻¹ of trimethoprim, and 8 mg l⁻¹ of nitisinone, given ad libitum.

### Viruses

YFV (17D, Asibi, Dakar, INH, INS, M185D and Kouma), WESSV, BANV, UGSV and ENTV viruses were obtained from World Reference Center for Emerging Viruses and Arboviruses (WRCEVA) at the University of Texas Medical Branch. DENV1 (Western Pacific 74), DENV2 (D2S20), DENV3 (16652), DENV4 (TVP 376), ZIKV (Dakar 41519), WNV (New York 1999 and Kunjin) and LGTV (TP21) viruses have been described previously. SINV-WEEV-McMillan GFP, SINV-VEEV-TrD GFP, SINV-SFV4 GFP, RRV T48-GFP and VEEV ZPC738 also were described previously. All viruses were propagated in Vero cells, and the MOI values used for target cells were calculated based on titration of infectivity in Vero cells.

### Gene Validation and Ectopic Expression

sgRNA against human LRP4 (5′-AGTGTGTACTCAGAGCGGTG-3′) (SEQ ID NO 1) or a non-targeting control were cloned into the LentiCRISPR-v2-puro backbone. Lentiviruses were generated by transfecting HEK293T cells with the transfer vector, psPAX2 and pMD2.G at a 2:2:1 ratio and collecting the supernatant after 48 h. HAP1 or HEK293T cells were transduced with lentiviruses and subjected to puromycin selection (2 μg ml⁻¹) for 7 days. Selected cells were cloned by limiting dilution. Single cloned cells were expanded and subjected to next-generation sequencing to confirm out-of-frame mutations and/or deletions of LRP4. ΔLRP1, ΔLRP4 or ΔVLDLR HepG2 or PHH cells were generated similarly as above, sgRNAs targeting each gene were cloned into the LentiCRISPR-v2-puro backbone. Lentiviruses were generated by transfecting HEK293T cells with the transfer vector, psPAX2 and pMD2.G at a 2:2:1 ratio and collecting the supernatant after 48 h. Cells were then selected with puromycin (1 μg ml⁻¹) for 7 days for HepG2 cells and 2-3 weeks for PHH. Surviving bulk cells were then collected and subjected to next-generation sequencing to confirm out-of-frame mutations and/or deletions. sgRNA sequences were as follows:

human LRP4
(SEQ ID NO 2)
(5′-GAGAACTGCAATGTTAACAA-3′);
human LRP1
(SEQ ID NO 3)
(5′-GCCGGCCCTTGCCATACACG-3′);
and
human VLDLR
(SEQ ID NO 4)
(5′-TCCTCACTCACCGGTTCCGG-3′).

cDNA encoding LRP4 (Homo sapiens, GenBank: NP_002325.2) residues 21-1905 was cloned with an N-terminal Flag tag into the pLKO-P2A puromycin lentiviral vector. The LRP4 ALBD construct containing residues 351-1905 of LRP4, the LRP4 AEGF Db-propeller construct lacking 354-1610 and the LRP4 LBD orthologues (C. jacchus, GenBank: XP_035118447, residues 26-350; E. caballus, GenBank: XP_023509751, residues 26-350; M. musculus, GenBank: NP_766256, residues 26-350; M. fascicularis, GenBank: XP_005578072, residues 26-350; M. mulatta, GenBank: XP_014969841, residues 26-350; M. nemestrina, GenBank: XP_011722772, residues 29-302; M. auratus, GenBank: XP_040601762, residues 26-350; S. boliviensis, GenBank: XP_010335713, residues 26-350; S. apella, GenBank: XP_032138026, residues 26-350) were ordered from Twist Biosciences and cloned into pTwist Lenti-SFFV vector. Lentiviruses were produced as described above and transduced into K562 cells. Transduced cells were selected for 7 days with puromycin (1 μg ml⁻¹, Invivogen) and Flag-positive cells were sorted for equivalent expression on a MACSQuant Tyto instrument (Miltenyi Biotec).

### LDLR Family Plasmid Generation

cDNA encoding LBD domains of LDLR family members (Table 1) were ordered from IDT and cloned into the pTwist-Lenti-SFFV-Puro-VLDLR vector, resulting in chimeric molecules in which the LBD of VLDLR was replaced with those of other LDLR family members. Expression was monitored by flow cytometry with an N-terminal Flag tag. Lentiviruses were produced as described above and transduced into K562 cells. Transduced cells were selected for 7 days with puromycin (1 μg ml⁻¹, Invivogen) and Flag-positive cells were sorted for equivalent expression on a MACSQuant Tyto instrument (Miltenyi Biotec).

### Reporter Virus Particle Generation

RVPs were produced by complementation of a GFP-expressing WNV replicon with plasmids encoding the structural genes of YFV-17D, as described previously with slight modifications. In brief, HEK293T cells were preplated in a low-glucose formulation of DMEM containing 25 mM HEPES (Invitrogen), 7% FBS and 100 U ml⁻¹ penicillin-streptomycin, transfected with plasmids encoding the replicon and structural genes at a 1:3 ratio with Lipofectamine 3000 (Invitrogen). For each microgram of DNA, 2 μl of Lipofectamine 3000 was used. Then, 6 h after transfection, fresh medium was changed, and the cells were maintained at 37 °C. The supernatant was collected at 48 h after transfection, passed through a 0.22-μm filter (Millipore), concentrated with an Amicon 100 kDa centrifugal filter (Millipore) and stored at −80 °C.

To titre the RVP infectious units (IU), the supernatants were diluted twofold and used to infect 10⁴ Raji-DC-SIGNR cells at 37 °C. Cells were fixed in 4% paraformaldehyde at 24 h after RVP infection, and GFP-positive cells were measured by flow cytometry. The virus titre was calculated using the linear portion of the virus-dose infectivity curve using the following formula: IU/sample volume=(percentage of GFP-positive cells)×(number of cells)× (dilution factor).

### Phylogenetic Analysis

The E protein sequences of DENV1 (NCBI: XDT81569.1), DENV2 (NCBI: XDT57287.1), DENV3 (NCBI: XDT57290.1), DENV4 (NCBI: XDT57288.1), ZIKV (NCBI: XDO24423.1), KUNV (NCBI: AOS89787.1), WNV-NY99 (NCBI: QXU69896.1), YFV-17D (NCBI: QLC45897.1), YFV-Asibi (NCBI: QWJ89576.1), Powassan virus (POWV) (NCBI: QRX38950.1), LGTV (NCBI: WEF36051.1), Sepik virus (SEPV) (NCBI: ABI54479.1), JEV (NCBI: BDH75798.1), WESSV (NCBI: UUB88388.1), BANV (NCBI: YP_009222007.1), UGSV (NCBI: YP_009344968.1), ENTV (NCBI: YP_950477.1), Bouboui virus (BOUV) (NCBI: YP_009344961.1) and Edge Hill virus (EHV) (NCBI: YP_009256192.1) were obtained from NCBI GenBank. Clustal Omega was used to align the sequences with simple phylogeny inferred through neighbor-joining. Results were visualized in R using the ggtree package.

### Alignment of LRP4 Orthologues

The LBD domain sequences of LRP4 from human (NCBI: NP_002325.2), C. jacchus (NCBI: XP_014969841.2), E. caballus (XP_023509751.1), M. musculus (NCBI: NP_766256.3), M. fascicularis (NCBI: XP_005578072.1), M. mulatta (NCBI: XP_014969841.2), M. nemestrina (NCBI: XP_011722772.1), M. auratus (NCBI: XP_040601762.1), S. boliviensis (NCBI: XP_010335713.2), S. apella (NCBI: XP_032138026.1) were obtained from NCBI GenBank. Sequences were aligned with the ClustalW algorithm using the msa R package.

CRISPR-Cas9 sgRNA Library Design, Screening and Analysis

A list of 1,146 surface proteins was obtained based on datasets for plasma membrane proteins. Four sgRNAs targeting each gene were selected to produce a genome-wide Brunello CRISPR-KO library, and 50 non-targeting control sgRNAs were included. sgRNA guide sequences were cloned into lentiCRISPRv2-puro (Addgene, 52961) by the Genome Engineering and iPSC core (GEiC) at Washington University. The sgRNA plasmid library was packaged in HEK293T cells with psPAX2 (provided by D. Trono, Addgene, 12260) and pMD2.G (provided by D. Trono, Addgene, 12259) using TurboFectin 8.0 transfection reagent according to the manufacturer's instructions. The supernatants were collected 2 days after transfection, pooled, clarified by centrifugation (1,200 rpm for 5 min), filtered through a 0.45 μm membrane and stored at −80 °C.

Here, 5×10⁷ HAP1 ΔHS cells were transduced at an MOI of 0.1 to 0.3 with the packaged lentiviral library and selected cells with puromycin (2 μg ml⁻¹) for 7-10 days. Then 2×10⁷ cells were inoculated with YFV-17D at an MOI of 10. The surviving cells were expanded, and cell pellets frozen at −80 °C. Genomic DNA from the control and selected cell pellets were prepared using the Monarch Genomic DNA Purification Kit (NEB). The guide sequences were amplified from genomic DNA using a pooled forward primer set (5′-AATGATACGGCGACCACCGAGATCTACACCTGATGACACTCTTTCCCTACA CGACGCTCTTCCGATCTN₁₋₆TTGTGGAAAGGACGAAACACCG-3′) (SEQ ID NO 5) and a barcoded reverse primer (5′-CAAGCAGAAGACGGCATACGAGATN₈GTGACTGGAGTTCAGACGTGTGCTC TTCCGATCTCCAATTCCCACTCCTTTCAAGACCT-3′) (SEQ ID NO 6). The amplified product was purified by gel extraction and subject to (1×150 bp) sequencing on an Illumina MiSeq instrument. sgRNA sequences were extracted from the demultiplexed FASTQ files, and enriched genes were identified compared to control library cells with the MAGeCK package.

### In Vitro Infection Experiments

HAP1 ΔHS cells and HEK293T cells were inoculated with YFV-17D at an MOI of 1 (HAP1 ΔHS) or 20 (HEK293T) for 16 h before analysis. K562 cells were inoculated with YFV-17D at an MOI of 1 for 24 h. Wild-type HAP1, HEK293T and Vero cells were infected with YFV-17D at an MOI of 20 (HEK293T) or 1 (HAP1 or Vero) for 24 h. HepG2 cells were inoculated with YFV-17D at an MOI of 2 for 24 h or MOI of 5 for 16 h. Cells were stained with a 1:1 mixture of Orthoflavivirus cross-reactive 4G2 and E60 mAbs at 1 μg ml⁻¹ for analysis on an iQue flow cytometer. Data were analyzed and normalized in the figures.

### Multi-Step Viral Growth Analysis

Control or transduced K562 cells were incubated with YFV-17D at an MOI of 0.01 for 2 h at 37 °C. Subsequently, unbound virus was removed after three washes with K562 maintaining medium, and the cells were resuspended in fresh K562 maintaining medium. Culture supernatants were collected at 12, 24, 36, 48, 60 and 72 h. The supernatant was immediately frozen and subsequently, after thawing, titred on Vero cells by a focus-forming assay.

### PHH Infection Experiments

PHHs were generated. The human patient was a 35-year-old male individual with hepatocellular carcinoma, and the liver tissue was collected from a clearly defined, unaffected region. To establish organoid cultures, biopsy tissue was placed in Liver Isolation medium on ice and then plated into wells of a 24-well plate and fed with 50% L-WRN-cell-conditioned medium+liver isolation reagents. Organoid cultures were passaged approximately 7 days later, at which point they were switched to liver establishment medium (LE). Organoids were passaged approximately every 7 days for the remainder of culture, throughout expansion for experimental needs. Cells were then transferred to Transwell 24-well plates and cultured in LE medium until they were switched into differentiation medium 4 days later. Organoids were kept in differentiation medium until day 11. Cells were then inoculated with YFV-M185D at an MOI of 10 for 48 h in the presence of ruxolitinib (10 μM). Cells were analysed for YFV infection using flow cytometry.

### Virus Attachment and Internalization Assays

To assess virus attachment to the cell surface, YFV-17D (MOI of 0.1) was incubated with control or LRP4-expressing K562 cells on ice for 1 h in K562 maintaining medium. Unbound virus was removed by washing six times with ice-cold binding buffer (Hank's balanced salt solution supplemented with 2 mM CaCl₂) and 2% BSA). RNA was extracted using the Zymo Quick-RNA MicroPrep Kit (R1050). Viral (F: 5′-AGGTGCATTGGTCTGCAAAT-3′ (SEQ ID NO 7), R: 5′-TCTCTGCTAATCGCTCAACG-3′) (SEQ ID NO 8) and GAPDH (Hs.PT.39a.22214836) RNA levels were determined using a TaqMan RNA-to-Ct 1-Step Kit on the QuantStudio 6 system.

To assess virus internalization, after removing unbound virus, cells were incubated at 37 °C for 1 h to allow for internalization. Cells were resuspended in PBS supplemented with 0.5 mg ml⁻¹ of proteinase K (Sigma-Aldrich, P2308) and 100 μg ml⁻¹ RNase A (Thermo Scientific, EN0531) for 1 h on ice to digest bound but uninternalized virus. After three washes with PBS, RNA was then extracted using the Zymo Quick-RNA MicroPrep Kit (R1050), vRNA and GAPDH RNA levels were determined using a TaqMan RNA-to-Ct 1-Step Kit on the QuantStudio 6 (Thermo Fisher Scientific) system.

### Protein Production

LDLRAD3-LA1-Fc, YFV-136 IgG and DC2.112 IgG were produced. The LBD (LA1-8) of human LRP4 (residues 21-350), LRP4 LA truncations (single LA domains (LA1-8), tandem LA domains (LA(1-2, 3-4, 5-6 or 7-8), triple LA domains (LA(1-3) to LA(6-8)), LA(2,6,7), LA(2,3,6,7), LA(2-4)-3×, LRP1_CL-I (GenBank: NP_002323.2, residues 25-110), VLDLR-LBD (GenBank: NP_003374.3, residues 31-355) and VLDLR-LA(1-2) all were subcloned into a pTwist CMV β-globin expression vector with an upstream mouse IgH secretion signal and a downstream HRV 3C cleavage site linked to a human lgG Fc sequence by a GGGSGGS linker. To express recombinant proteins, constructs were co-transfected with human LRPAP1 (RAP) chaperone gene cDNA (residues 1-353, which removes the C-terminal ER retention signal, HENL) at a 1:1 ratio with Expifectamine 293 reagent into Expi293 cells (Thermo Fisher Scientific) according to the manufacturer's instructions. The supernatants were collected 5 days after transfection, centrifuged (6,000 g) and passed through a 0.22-mm filter. Fc-fusion proteins were bound to PrismA protein A resin (Cytiva) using a gravity-flow column. The column was washed with 25 column volumes of 1×TBS-EDTA (50 mM Tris pH 8.0, 150 mM NaCl, 10 mM EDTA), 50 column volumes of high-salt buffer (50 mM Tris pH 8.0, 500 mM NaCl, 10 mM EDTA) to strip RAP, followed by 25 column volumes of 1×TBS+10 mM CaCl₂). Proteins were eluted with 1 M L-arginine pH 4.0 and dialyzed with a Thermo Fisher Scientific Slide-A-Lyzer 10 kDa MWCO into 1×HBS-C(25 mM HEPES pH 8.0, 150 mM NaCl, 2 mM CaCl₂). After dialysis, protein was concentrated with an Amicon 10 kDa MWCO centrifugal filter (Millipore). The protein purity was assessed by reducing and non-reducing SDS-PAGE followed by staining with SimpliSafe Coomassie reagent (Thermo Fisher Scientific). Gels were imaged with an iBright 1500 instrument (Thermo Fisher Scientific). Biotinylation of proteins and antibodies was performed using the EZ-Link NHS-PEG4-Biotinylation kit (Thermo Fisher Scientific) according to the manufacturer's protocol.

Wild-type or mutant trimeric E-DIII (YFV-Asibi; GenBank: KF769016.1) was subcloned into a pTwist CMV β-globin expression with an engineered C-terminal fibritin trimerization domain and a TwinStrep tag for purification. To express recombinant proteins, constructs were transfected with Expifectamine 293 reagent into Expi293 cells according to the manufacturer's instructions. Supernatants were collected 5 days after transfection, centrifuged (6,000 g) and passed through a 0.22-mm filter. The supernatants were applied to StrepTactin XT (IBA Lifesciences) resin and purified on a gravity flow column. The column was washed with 20 column volumes of 1×buffer W (100 mM Tris pH 8.0, 150 mM NaCl, 1 mM EDTA). Proteins were eluted with 10 column volumes of 1× buffer BXT (100 mM Tris/HCl, pH 8.0, 150 mM NaCl, 1 mM EDTA, 50 mM biotin). Protein was then dialyzed and buffer exchanged with concentrated with an Amicon 10 kDa MWCO centrifugal filter.

A cDNA encoding E-DIII (YFV-Asibi; GenBank: KF769016.1) was inserted into a pET-21 vector. The protein was expressed in BL21 (DE3) cells at 37 °C as insoluble inclusion bodies. Protein expression was induced with 1.0 mM IPTG for 6 h after the cells reached an OD600 of 0.8. The cells were collected, and the inclusion bodies were isolated and washed. The inclusion bodies were denatured and then refolded by rapid dilution in a buffer that promoted formation of disulfide bonds. The refolding buffer consisted of 0.4M non-detergent sulfobetaine (NDSB)-201, 100 mM Tris-HCl (pH 8.0), 2.0 mM EDTA (pH 8.0), 5.0 mM L-glutathione reduced, 0.5 mM L-glutathione oxidized, and 0.5 mM 4-(2-aminoethyl)benzenesulfonyl fluoride hydrochloride (AEBSF) inhibitor. The refolded protein was concentrated, and aggregates were removed using a Superdex S200 Increase column. Correct folding was confirmed by reactivity against the anti-YFV mAb Adi-49147.

### ELISA

Nunc MaxiSorp ELISA plates (Thermo Fisher Scientific) were coated with E60 mAb (2 μg ml⁻¹) or LDLRAD3 LA1-Fc (negative control) and LRP4-Fc proteins (start at 30 μg ml⁻¹ with threefold dilution) overnight in sodium bicarbonate buffer, pH 9.3. Plates were washed with PBS and blocked with PBS containing 2% (w/v) BSA for 1 h at room temperature. YFV-17D (106 FFU) was diluted in PBS containing 1% BSA and added to wells for 2 h at room temperature. LDLRAD3-Fc (negative control) and LRP4-Fc proteins (start at 10 μg ml⁻¹ with threefold dilution) or E60 anti-E protein mAb (5 μg ml⁻¹) were diluted in 1% BSA and incubated for 2 h at room temperature. The plates were washed with PBST and incubated with horseradish-peroxide-conjugated goat anti-mouse IgG or horseradish-peroxide-conjugated goat anti-human IgG (1:2,000, Jackson ImmunoResearch) for 1 h at room temperature. The plates were developed 3,3′-5,5′-tetramethylbenzidine substrate (Thermo Fisher Scientific), stopped with 2 N H₂SO₄ and read at 450 nM using the TriStar Microplate Reader (Berthold).

To measure Fc fusion proteins levels in vivo, Nunc MaxiSorp ELISA plates (Thermo Fisher Scientific) were coated with serum from injected mice in coating buffer (BioLegend, 421701) (starting at 5 μl with serial fivefold dilutions). The same amount of serum from naive animals was added to coating buffer as a comparison to exclude non-specific signal. LDLRAD3-LA1-Fc, LRP4-LBD-Fc, VLDLR-LA(1-2)-Fc, LRP1_CL-I-Fc and other Fc decoy proteins were run separately on the same plate as standards with known concentrations (starting at 10 μg ml⁻¹ with threefold serial dilutions in naive serum). The plates were washed with PBS and blocked with PBS containing 2% (w/v) BSA for 1 h at room temperature. After additional washing with PBST, plates were incubated with horseradish-peroxide-conjugated goat anti-human IgG (1:2,000, Jackson ImmunoResearch) for 1 h at room temperature. Subsequently, after washing with PBST, the plates were developed 3,3′-5,5′-tetramethylbenzidine substrate (Thermo Fisher Scientific), and the reaction was stopped with 2 N H₂SO₄. Signals were read at 450 nM using a TriStar Microplate Reader (Berthold).

### BinJV-YFV-prME Chimeric Virus Generation

YFV-BinJV (prM-E) chimeric viruses were generated with a modified circular polymerase extension reaction (CPER) protocol. Overlapping fragments of the BinJV cDNA genome (GenBank: MG587038.1) and the YFV prM-E genes (YFV M185D (GenBank: KU978764.1), Asibi (GenBank: KF769016.1)) and ES504 (GenBank: KY885000.2) were ordered from Twist Biosciences in the pTwistAmp High Copy vector. Each fragment was amplified from a carrier plasmid using the Q5 High-Fidelity DNA polymerase (New England Biolabs), followed by Dpnl digestion and gel extraction of the PCR products. The fragments were assembled to form the circular dsDNA with a modified CPER protocol. The circular CPER product was then transfected into C6/36 Aedes albopictus cells using FlyFectIN (OZ Biosciences) according to the manufacturer's instructions.

For virion purification, YFV-BinJV chimeric viruses were collected at 7 and 10 days after infection. The supernatants were clarified by centrifugation and filtered through a 0.2-μm filter. The particles were precipitated overnight with PEG 8000 (final concentration of 8%) while stirring, pelleted by centrifugation (6,000 g for 45 min) resuspended in TNE buffer (25 mM Tris-HCl pH 8, 150 mM NaCl, 2 mM EDTA), and then ultracentrifuged through a 25% sucrose cushion (SW32Ti rotor, 32,000 RPM for 2 h). The resulting pellet was resuspended overnight in 1 ml of TNE, and then virions were purified by gradient ultracentrifugation using a 10-40% potassium tartrate gradient (SW32Ti rotor at 32,000×RPM for 2 h). The visible virus band was extracted, and buffer-exchanged into TNE by ultrafiltration.

### Imaging of Chimeric YFV-M185D/BinJ and YFV-Asibi/BinJ Virions

YFV-M185D/BinJ and YFV-Asibi/BinJ samples (absorbance at 280 nm, 3.0) were applied to glow-discharged lacey carbon grids (Ted Pella, 01895-F) and flash-frozen in liquid ethane using a Vitrobot Mark IV (Thermo Fisher Scientific). Grids were loaded into a Cs-corrected FEI Titan Krios 300 kV microscope or an FEI Glacios 200 kV microscope, each equipped with a Falcon 4 direct electron detector, and then imaged at a magnification of ×59,000 (Krios) or ×120,000 (Glacios), resulting in a calibrated pixel size of 1.1 Å (Krios) or 1.184 Å (Glacios). Videos were collected in EER format with a total dose of around 50-57 e⁻ Å⁻² per video (around 7 e⁻ Å² s⁻¹ over 8-9 s acquisition).

### BLI Analysis

Binding experiments were performed on a GatorPlus BLI and analysed using GatorOne (v.2.15) software. All experiments were performed with 1×HBS (25 mM HEPES pH 8.0, 150 mM NaCl) supplemented with 1% BSA and 2 mM CaCl₂). To evaluate binding of LRP4, LRP1 or VLDLR proteins in the solid phase, anti-human IgG Fc coated pins were incubated with around 50 μg ml⁻¹ of LRP4, LRP1 or VLDLR Fc fusion proteins for 60 s. After washing in running buffer for 120 s, YFV-BinJV virions (50 μg ml⁻¹) were captured for 900 s. To evaluate binding of LRP4 protein in solution, streptavidin-coated pins were incubated with biotinylated YFV-136 mAb (50 μg ml⁻¹) to enable capture of YFV-BinJV virions (50 μg ml⁻¹). After washing in running buffer for 120 s, LRP4 proteins (50 μg ml⁻¹) were associated for 900 s. To evaluate binding of LRP4 protein in the solid phase with YFV-Asibi E-DIII protein, anti-human IgG Fc was incubated with 50 μg ml⁻¹ of LRP4-LBD-Fc for 60 s. After washing in running buffer for 120 s, 500 μg ml⁻¹ of YFV-Asibi E-DIII monomeric protein was added for 900 s.

### SPR Analysis

SPR binding experiments were performed on a Biacore T200 system using its BioEvaluation software (GE Healthcare). LRP4-LBD-Fc and LDLRAD3-LA1-Fc (a negative reference control) were immobilized onto separate channels of a CM5 sensor chip (GE Healthcare) using standard amine coupling chemistry. Experiments were performed using HBS (0.01 M HEPES pH 7.4, 0.15 M NaCl) supplemented with 1 mM CaCl₂) as running buffer with refolded monomeric YFV E-DIII injected over a range of concentrations (320 UM to 1.3 μM). Preliminary experiments demonstrated slow dissociation kinetics, necessitating hours of contact time to achieve steady-state equilibrium at lower concentrations of YFV E-DIII. As such, experiments were performed at 1 μl min-1 to enable an association period of 5 h, which was followed by a 1 h dissociation period. LDLRAD3-LA1-Fc reference-subtracted binding curves were exported and analysed using a custom Python script. The kinetic binding analysis used a 1:2 site model, which modelled each interaction using the integrated rate equation. For equilibrium binding analysis, the maximum response at each concentration was first normalized by the sensor's overall maximum response. The equilibrium affinity parameters were then fitted using the following equation:

$$Normalized R U = r \frac{[ L ]}{[ L ] + {K}_{D}^{site 1}} + ( 1 - r ) \frac{[ L ]}{[ L ] + {K}_{D}^{site 2}}$$

Where [L] corresponds to the concentration of YFV E-DIII, and r corresponds to the ratio of the maximum response produced by binding to site 1 (and therefore 1-r corresponds to the ratio of the maximum response produced by binding to site 2). The Hill-Langmuir analysis used this equation directly, whereas the Scatchard analysis also divided the normalized response by [L].

### Neutralization Assays

To assess the neutralization capacity of Fc-fusion proteins, wild-type HAP1, HEK293T, Vero or HepG2 cells were plated (2×10⁴ per well) in a 96-well plate and were allowed to adhere overnight in DMEM supplemented with 10% FBS. The next day, YFV was incubated with the indicated LRP4, VLDLR, LRP1 or LDLRAD3 Fc-fusion proteins for 1 h at 37 °C in DMEM with 2% FBS before inoculation of cells. At 24 h.p.i., cells were collected and analysed using flow cytometry with a mixture of 4G2 and E60 anti-E protein mAbs. For RAP blocking experiments, HepG2 cells were plated (2×10⁴ per well) in a 96-well plate and adhered overnight in DMEM supplemented with 10% FBS. The next day, the medium was changed to DMEM supplemented with 2% FBS containing RAP-D3 or Mut-RAP-D3 proteins for 30 min before YFV-17D infection. Cells were collected at 16 h.p.i. and analysed using flow cytometry using 4G2 and E60 mAbs on the iQue3 Flow Cytometer (Sartorius) and using FlowJo (v.10) software.

### Mouse Experiments

For decoy treatment experiments, 500 μg of LRP4-LA(2-4)-3×-Fc, LRP4-LA(2,3,6,7)-Fc, LRP1-CL_I-Fc, VLDLR-LA(1-2)-Fc or LDLRAD3-LA1-Fc proteins was incubated with 10⁵ PFU of YFV-17D at 37 °C for 1 h and then inoculated into AG129 mice or C57BL/6J mice treated with 2 mg of anti-IFNAR1 (MAR1-5A3) mAb (Bio X Cell) through intraperitoneal injection. Mice were also given 500 μg of the same proteins at 1 and 2d.p.i. At 3 d.p.i., mice were euthanized, perfused with PBS (25 ml) and tissues were collected for storage at −80 °C before processing.

In separate experiments, wild-type and Lrp4^{tm1Her}/J mice were given 2 mg of anti-IFNAR1 mAb through intraperitoneal injection. One day later, mice were inoculated with 10⁵ PFU of YFV-M185D through intraperitoneal infection. Then, at 5 d.p.i., the mice were euthanized, perfused with PBS (25 ml) and tissues were collected for storage at −80 °C before processing. For VEEV infection experiments, wild-type and Lrp4^{tm1Her}/J mice were inoculated subcutaneously in the footpad with 10² FFU of VEEV-ZPC738 in a 50 μl volume. Then, at 4 d.p.i., the mice were euthanized, perfused with PBS and tissues were collected for storage at −80 °C before processing.

For hFRG mouse experiments, hFRG mice (aged 24-25 weeks; obtained from Yecuris) were administered 50 mg per kg doses of LRP1_CL-I-Fc or LDLRAD3-LA1-Fc decoy through intraperitoneal injection 6 h before and 24 h after intraperitoneal inoculation with 2×10⁵ FFU of YFV-Dakar. Weight was monitored daily and, at 5 d.p.i., the mice were euthanized, perfused with PBS and tissues were collected for storage at −80 °C before processing.

### Serum ALT Assay

Serum ALT level was quantified using a commercial ALT reagent kit (Teco Diagnostics) according to the manufacturer's instructions with modification to a 96-well format. In brief, 10 μl of serum was mixed with 50 μl of ALT substrate and incubated at 37 °C for 30 min. After a 10-min incubation at 37 °C, 200 μl of ALT color developer was added, followed by a 5-min incubation at 37 °C. The plate was read on a plate reader (CLARIOstar, BMG Labtech) at 505 nm.

### Prothrombin Time

Prothrombin time was measured from whole blood using the CoaguChek XS System (Roche) according to the manufacturer's instructions.

### Viral RNA Analysis

Tissues were homogenized with a MagNA Lyser (Roche), and viral RNA was extracted with a MagMAX Viral RNA extraction kit using a Kingfisher Flex instrument (Thermo Fisher Scientific) according to the manufacturer's instructions. Viral RNA levels were determined using the TaqMan RNA-to-Ct 1-Step Kit on the QuantStudio 6 (Thermo Fisher Scientific) system using the following primer and probe sets: YFV (F: 5′-AGGTGCATTGGTCTGCAAAT-3′, (SEQ ID NO 9) R: 5′-TCTCTGCTAATCGCTCAACG-3′, (SEQ ID NO 10) YFV probe 5′/56-FAM/AGGCAATAA/ZEN/ACACATTTGGA/3IABKFQ/-3′); (SEQ ID NO 11) VEEV (F: 5′-CAAGTCGAGGCAGACATTCA-3′ (SEQ ID NO 12), R: 5′-CAGGGTGTCAAGGATGGATAAA-3′, (SEQ ID NO 13) VEEV ZPC738 probe: 5′-/56-FAM/TGGTCCATT/ZEN/CCTCATGCATCCGAC/3IABKFQ/-3′) (SEQ ID NO 14). FFU equivalents were determined by parallel processing of a viral stock with known titre.

### Statistical Analysis

Statistical significance was determined using Prism v.10.1.1 (GraphPad); P<0.05 was considered to be significant. The statistical tests, number of independent experiments and number of experimental replicates are indicated in the figure descriptions.

TABLE 1
LDLR family member sequences

| Name | Sequence |
|---|---|
| LDLR | ATGGACTACAAGGACGACGATGACAAGGGATCCGACAGATGTGAGCGAAATGAGTTCCA |
| | GTGCCAAGATGGAAAATGCATATCCTACAAGTGGGTCTGTGATGGTTCAGCGGAGTGCCA |
| | GGATGGCTCTGACGAGTCCCAAGAAACGTGTCTTTCTGTGACTTGTAAGAGCGGCGATTT |
| | TAGCTGCGGCGGCAGGGTCAACAGGTGTATTCCCCAGTTTTGGCGCTGCGACGGGCAG |
| | GTTGACTGCGACAATGGAAGCGACGAGCAGGGTTGTCCGCCAAAAACCTGTAGTCAGGA |
| | TGAATTTAGATGCCACGATGGAAAATGTATATCTCGACAATTTGTGTGCGACTCTGACCGA |
| | GACTGCCTGGACGGTAGTGACGAAGCGTCCTGCCCCGTATTGACATGTGGACCGGCCAG |
| | CTTTCAGTGTAACTCTAGCACCTGTATTCCTCAACTGTGGGCTTGTGATAATGATCCTGAC |
| | TGCGAAGATGGGTCAGACGAATGGCCTCAGAGATGCCGGGGACTTTATGTTTTTCAAGGT |
| | GATTCCTCCCCTTGCAGCGCATTTGAATTTCACTGCCTCTCAGGTGAGTGCATACATAGCA |
| | GCTGGCGCTGCGACGGGGGGCCTGATTGCAAGGATAAGTCTGACGAAGAGAACTGCGC |
| | TGTAGCTACCTGTAGGCCAGATGAATTCCAGTGCTCAGACGGGAATTGTATTCACGGTAG |
| | TCGCCAGTGTGACCGCGAGTACGATTGCAAAGACATGTCAGACGAGGTAGGCTGCGTAA |
| | ATGTTACGCTTTGCGAGGGACCAAACAAATTTAAGTGCCATTCCGGTGAATGCATAACGC |
| | TGGACAAGGTCTGCAACATGGCGAGAGATTGTCGCGATTGGAGCGACGAACCTATTAAG |
| | GAGTGTGGAGGATCCCATATAAACGAATGCTTGGTAAATAATGGT (SEQ ID NO 15) |
| LRP1_ | ATGGACTACAAGGACGACGATGACAAGGGATCCAAAACGTGCTCACCCAAGCAATTCGCA |
| CL-I | TGCCGCGACCAAATCACTTGTATATCCAAGGGATGGAGGTGTGACGGTGAGCGAGACTG |
| | CCCCGATGGTTCCGACGAGGCGCCTGAGATCTGTCCACAATCTAAAGCCCAACGCTGCC |
| | AACCTAACGAGCACAATTGCTTGGGTACTGAACTTTGCGTCCCCATGTCTCGCCTTTGTAA |
| | TGGTGTCCAAGATTGCATGGACGGGAGCGACGAGGGGCCACATTGTAGAGAAGGAGGAT |
| | CCCATATAAACGAATGCTTGGTAAATAATGGT (SEQ ID NO 16) |
| LRP1_ | ATGGACTACAAGGACGACGATGACAAGGGATCCCCGCAGTGCCAGCCAGGCGAATTCGC |
| CL-II | TTGTGCCAACTCTCGCTGTATACAGGAGCGCTGGAAATGTGATGGCGATAACGATTGCTT |
| | GGATAATTCCGATGAGGCACCAGCGCTTTGTCATCAACATACATGCCCCAGCGATCGCTT |
| | TAAATGCGAAAACAACAGATGCATCCCCAACAGGTGGCTTTGTGATGGTGATAACGATTG |
| | CGGTAACTCTGAAGATGAATCCAACGCTACCTGTTCTGCGCGGACGTGTCCGCCCAACCA |
| | ATTCTCCTGTGCATCCGGCCGATGTATTCCTATCTCATGGACTTGTGATCTTGACGACGAT |
| | TGTGGCGATCGATCTGATGAATCCGCGAGCTGTGCATATCCAACGTGTTTCCCGCTGACG |
| | CAATTCACTTGCAATAATGGCAGGTGTATCAATATTAATTGGCGATGCGATAATGACAATG |
| | ACTGTGGTGATAACTCTGATGAAGCAGGCTGCAGCCACTCCTGTTCTTCAACTCAGTTTAA |
| | ATGCAATTCAGGAAGGTGTATCCCAGAACATTGGACTTGTGATGGCGACAACGATTGCGG |
| | CGACTATTCTGATGAAACACACGCGAACTGCACGAATCAGGCGACGCGACCCCCCGGCG |
| | GGTGTCACACGGACGAATTCCAGTGTCGCCTGGATGGCTTGTGTATTCCGCTCCGATGG |
| | CGATGTGACGGCGACACCGATTGCATGGATTCCTCCGATGAAAAATCCTGCGAAGGCGT |
| | GACACACGTTTGCGACCCGAGTGTGAAATTCGGCTGTAAGGACAGCGCCCGGTGTATTA |
| | GCAAGGCGTGGGTGTGCGATGGGGACAATGACTGCGAGGACAATTCTGATGAAGAGAAT |
| | TGCGAGTCTTTGGCATGTCGCCCCCCGTCTCATCCATGCGCCAACAATACATCTGTCTGT |
| | TTGCCCCCTGACAAGCTTTGTGACGGAAATGACGACTGCGGCGACGGGTCAGACGAAGG |
| | GGAATTGTGTGGAGGATCCCATATAAACGAATGCTTGGTAAATAATGGT (SEQ ID NO 17) |
| LRP1_ | ATGGACTACAAGGACGACGATGACAAGGGATCCAGTTCCTGTCGAGCGCAGGATGAGTT |
| CL-III | CGAATGCGCGAATGGAGAGTGTATTAATTTTAGTCTCACTTGCGATGGCGTACCCCATTG |
| | TAAAGACAAAAGCGATGAAAAACCTTCATACTGCAATTCCCGGCGATGCAAGAAAACCTTC |
| | CGGCAGTGTTCTAATGGTAGGTGCGTGTCAAACATGCTTTGGTGCAACGGAGCGGACGA |
| | TTGCGGCGATGGATCCGACGAGATCCCTTGTAATAAGACCGCATGCGGTGTCGGCGAGT |
| | TTCGGTGTCGGGATGGAACCTGTATAGGAAACTCTTCTCGGTGCAATCAGTTCGTTGACT |
| | GTGAAGACGCGTCCGATGAAATGAACTGCTCCGCCACCGACTGTAGCTCATATTTTCGAC |
| | TCGGAGTGAAAGGTGTGCTCTTTCAACCGTGTGAACGCACCAGTCTGTGCTATGCCCCCT |
| | CTTGGGTCTGTGATGGAGCTAACGATTGCGGCGATTACAGTGATGAGCGCGACTGTCCC |
| | GGGGTCAAACGCCCCCGCTGTCCCTTGAACTATTTTGCATGTCCTTCAGGCAGATGTATT |
| | CCGATGTCATGGACTTGCGACAAAGAGGATGACTGTGAACATGGAGAGGACGAAACGCA |
| | CTGTAACAAGTTCTGCTCCGAAGCTCAATTCGAATGTCAAAACCACAGGTGTATCAGTAAA |
| | CAGTGGCTTTGTGACGGTAGTGATGACTGCGGGGATGGATCTGATGAAGCAGCCCATTG |
| | CGAGGGTAAGACATGTGGGCCTTCTTCCTTCTCTTGTCCAGGCACGCATGTCTGTGTCCC |
| | AGAGAGATGGCTCTGCGACGGAGATAAGGATTGTGCAGACGGGGCTGATGAATCTATCG |
| | CTGCAGGATGTCTCTATAATAGTACCTGTGATGACAGAGAATTCATGTGCCAAAATCGACA |
| | GTGCATTCCCAAGCACTTTGTCTGCGATCACGACCGAGACTGTGCCGACGGGTCTGATG |
| | AATCTCCCGAGTGTGAATACCCAACCTGTGGCCCATCAGAATTTCGATGCGCTAACGGTC |
| | GCTGCCTTTCTTCTCGGCAGTGGGAATGCGATGGGGAGAACGACTGTCACGACCAAAGT |
| | GATGAGGCTCCTAAAAATCCACACTGCACGAGTCAAGAACATAAATGCAATGCATCAAGT |
| | CAGTTTCTTTGTAGTTCTGGCCGGTGCGTGGCAGAAGCGCTGCTTTGCAACGGACAAGAT |
| | GATTGCGGAGATAGCAGCGACGAAAGGGGATGTCATGGAGGATCCCATATAAACGAATG |
| | CTTGGTAAATAATGGT (SEQ ID NO 18) |
| LRP1_ | ATGGACTACAAGGACGACGATGACAAGGGATCCTCA AAT TGT ACT GCC TCC CAG TTC |
| CL-IV | GTG TGC AAG AAT GAT AAG TGT ATC CCA TTC TGG TGG AAA TGT GAT ACA GAG GAT |
| | GAC TGC GGG GAT CAT TCC GAC GAG CCC CCG GAT TGC CCG GAA TTT AAG TGT |
| | CGC CCA GGG CAA TTT CAG TGT AGT ACT GGT ATA TGC ACT AAC CCA GCC TTC ATA |
| | TGT GAT GGA GAT AAT GAC TGC CAA GAT AAC TCT GAC GAG GCG AAC TGC GAT |
| | ATA CAT GTA TGT CTT CCG AGT CAG TTC AAG TGC ACT AAT ACG AAT CGC TGT ATT |
| | CCA GGC ATC TTT AGA TGT AAT GGT CAG GAC AAC TGC GGC GAT GGA GAG GAT |
| | GAG AGA GAC TGC CCA GAG GTG ACT TGC GCT CCT AAT CAG TTT CAA TGT AGC |
| | ATA ACC AAA AGA TGT ATC CCG CGA GTT TGG GTG TGT GAC AGG GAC AAT GAC |
| | TGC GTC GAT GGA AGT GAT GAG CCC GCG AAC TGC ACC CAA ATG ACG TGT GGT |
| | GTA GAC GAG TTT CGC TGT AAG GAC AGC GGG AGA TGT ATC CCG GCC CGA TGG |
| | AAG TGC GAT GGT GAA GAC GAT TGC GGT GAT GGA TCA GAT GAG CCT AAG GAG |
| | GAG TGC GAC GAA CGC ACT TGC GAG CCG TAC CAA TTC CGG TGC AAG AAC AAC |
| | AGG TGT GTA CCC GGC CGC TGG CAG TGT GAC TAT GAT AAC GAT TGT GGG GAC |
| | AAT AGC GAC GAA GAG TCT TGT ACT CCC AGG CCA TGT TCT GAA TCC GAG TTC |
| | TCA TGT GCC AAT GGA AGG TGT ATC GCC GGG CGG TGG AAA TGT GAC GGT GAC |
| | CAT GAC TGC GCG GAT GGC TCC GAT GAA AAA GAT TGT ACC CCT CGG TGC GAT |
| | ATGGAC CAG TTC CAG TGT AAA TCC GGA CAT TGT ATA CCA TTG CGG TGG AGG |
| | TGTGAC GCT GAT GCA GAC TGT ATG GAT GGT AGT GAT GAA GAG GCC TGC |
| | GGTACA GGT GTT CGC ACA TGC CCT TTG GAC GAG TTC CAA TGC AAT AAC ACC CTT |
| | TGT AAG CCG CTG GCT TGG AAA TGC GAT GGC GAA GAT GAT TGT GGT GAT AAT |
| | TCC GAT GAG AAT CCT GAA GAA TGT GCT AGG TTC GTT TGC CCG CCT AAC AGA |
| | CCT TTT CGG TGT AAG AAC GAC CGG GTT TGC TTG TGG ATA GGA CGG CAG TGT |
| | GATGGTACGGACAACTGTGGCGACGGTACTGACGAAGAAGACTGCGAGCCTCCAACCGC |
| | CCATACGACTCACTGTAAAGATAAGAAGGAGTTTCTTTGCCGCAACCAACGCTGTCTTAG |
| | CTCTTCACTGCGGTGTAATATGTTCGACGACTGCGGTGACGGCAGTGATGAGGAAGACT |
| | GTAGTGGAGGATCCCATATAAACGAATGCTTGGTAAATAATGGT (SEQ ID NO 19) |
| LRP1B_ | ATGGACTACAAGGACGACGATGACAAGGGATCCGAA TTT TTG TTG GCC CTT TTG ACC |
| CL-I | CTG AGC GGT TTG CTG CCA ATA GCG CGC GTT TTG ACT GTA GGA GCA GAT AGG GA |
| | C CAG CAA CTG TGT GAC CCT GGG GAG TTC CTT TGC CAT GAT CAC GTG ACG TGT |
| | GTT TCC CAA AGT TGG CTG TGT GAC GGC GAC CCA GAC TGC CCC GAT GAC AGT G |
| | AT GAG AGC CTC GAT ACA TGC CCG GAG GAG GTA GAA ATA AAG TGC CCC CTT AAC |
| | CAT ATA GCT TGT TTG GGA ACC AAC AAA TGT GTG CAT TTG TCC CAA CTG TGT AAC |
| | GGG GTG CTT GAT TGC CCC GAT GGT TAT GAT GAA GGT GTC CAC TGC CAGGGAGG |
| | ATCCCATATAAACGAATGCTTGGTAAATAATGGT (SEQ ID NO 20) |
| LRP1B_ | ATGGACTACAAGGACGACGATGACAAGGGATCCATC TGC AAA GCA GGT GAG TTT CGC |
| CL-II | TGT AAG AAC CGC CAT TGC ATA CAA GCT CGG TGG AAA TGC GAT GGG GAC GAC G |
| | AC TGT CTC GAC GGT AGC GAC GAG GAC AGC GTA AAC TGT TTT AAC CAT TCT TGT |
| | CCT GAT GAT CAA TTC AAG TGT CAA AAT AAT CGC TGC ATT CCA AAA AGG TGG TTG |
| | TGT GAC GGC GCC AAC GAC TGT GGC AGC AAT GAA GAT GAG AGC AAT CAG ACA T |
| | GC ACT GCT AGG ACG TGT CAA GTC GAC CAA TTT TCA TGC GGT AAC GGC CGC TGC |
| | ATC CCA AGG GCG TGG CTC TGC GAC AGA GAG GAT GAC TGC GGA GAT CAG ACC |
| | GAT GAG ATG GCA TCT TGC GAA TTT CCA ACT TGC GAA CCA CTC ACT CAA TTC GTT |
| | TGT AAG TCT GGA CGA TGC ATA AGT TCA AAA TGG CAC TGC GAT AGC GAT GAT GA |
| | T TGC GGA GAT GGA TCA GAC GAA GTA GGC TGC GTC CAT AGC TGT TTC GAT AAT C |
| | AG TTT AGG TGC AGC TCA GGG CGG TGC ATT CCT GGC CAC TGG GCC TGT GAT GG |
| | C GAT AAT GAT TGCGGC GAT TTC AGC GAT GAA GCG CAG ATT AAT TGT ACG AAG G |
| | AG GAG ATA CAC TCACCC GCG GGG TGT AAC GGG AAT GAG TTC CAA TGC CAC CCA |
| | GAT GGA AAT TGT GTTCCC GAC CTG TGG CGG TGT GAC GGA GAG AAA GAT TGC G |
| | AA GAC GGT AGC GATGAA AAA GGC TGT AAT GGA ACC ATC AGA CTG TGC GAC CAT |
| | AAA ACT AAG TTT TCTTGC TGG AGC ACA GGC CGA TGT ATC AAC AAA GCT TGG GTC |
| | TGC GAC GGC GATATA GAC TGC GAA GAT CAA AGT GAC GAG GAT GAT TGC GAC TC |
| | CTTTCTGTGCGGTCCACCAAAGCATCCTTGTGCTAACGATACGTCAGTTTGTCTTCAACCG |
| | GAAAAGTTGTGTAATGGGAAGAAAGATTGTCCAGATGGCTCCGATGAGGGTTATCTTTGC |
| | GATGGAGGATCCCATATAAACGAATGCTTGGTAAATAATGGT (SEQ ID NO 21) |
| LRP1B_ | ATGGACTACAAGGACGACGATGACAAGGGATCCAGC TGC AAT GCG TAC AGC GAG TT |
| CL-III | C GAG TGT GGA AAT GGC GAA TGC ATA GAC TAT CAG CTG ACT TGT GAC GGA ATA C |
| | CG CAT TGT AAA GAC AAG TCT GAC GAG AAG CTT CTG TAC TGC GAA AAC CGG TCA |
| | TGT CGC CGC GGT TTT AAG CCG TGC TAT AAC AGG AGG TGT ATC CCT CAC GGA AA |
| | G CTG TGC GAC GGT GAA AAT GAT TGT GGA GAT AAC TCA GAT GAA CTT GAT TGC A |
| | AA GTG AGT ACG TGT GCG ACG GTG GAA TTC AGA TGT GOG GAT GGC ACG TGC AT |
| | C CCC CGG TCA GCG AGA TGC AAC CAA AAC ATT GAC TGT GCA GAC GCG TCA GAT |
| | GAA AAG AAC TGC AAT AAC ACT GAT TGT ACT CAT TTT TAT AAA CTT GGC GTC AAA |
| | ACC ACG GGG TTC ATA CGA TGC AAC TCT ACC AGC CTC TGT GTA CTG CCC ACA TG |
| | G ATC TGT GAT GGC AGT AAC GAT TGC GGA GAC TAT TCT GAC GAA CTC AAA TGT C |
| | CG GTG CAA AAT AAA CAC AAG TGT GAA GAA AAC TAT TTT TCC TGT CCA TCC GGC |
| | CGG TGC ATC CTT AAT ACT TGG ATT TGC GAT GGT CAA AAG GAC TGC GAG GAC GG |
| | T CGG GAT GAG TTT CAC TGC GAC AGC TCC TGT TCC TGG AAC CAA TTC GCA TGT T |
| | CC GCA CAA AAG TGC ATC AGT AAG CAT TGG ATC TGT GAC GGA GAA GAT GAT TGT |
| | GGA GAT GGT CTT GAT GAA TCT GAC TCA ATC TGC GGG GCC ATT ACT TGC GCC GC |
| | A GAC ATG TTC AGT TGT CAA GGT TCC AGA GCA TGC GTC CCA CGA CAC TGG CTT T |
| | GT GAT GGA GAA AGG GAC TGC CCT GAT GGT AGC GAT GAA CTG TCA ACC GCC GG |
| | G TGC GCA CCT AAC AAC ACC TGT GAC GAA AAT GCT TTT ATG TGC CAC AAT AAA G |
| | TC TGT ATA CCC AAG CAG TTT GTA TGT GAT CAC GAT GAC GAT TGC GGA GAC GGA |
| | TCT GAC GAA AGT CCC CAG TGT GGC TAC CGA CAA TGC GGT ACG GAA GAG TTC TC |
| | T TGC GCT GAT GGC CGC TGC CTC CTC AAC ACG CAA TGG CAG TGC GAC GGT GAT |
| | TTC GAT TGT CCA GAT CAC TCT GAC GAA GCC CCA CTT AAC CCA AAA TGC AAA TCC |
| | GCG GAA CAG TCA TGT AAT AGT TCA TTC TTT ATG TGT AAG AAC GGC AGG TGT ATA |
| | CCG AGT GGC GGA CTC TGC GAC AAT AAA GAC GATTGCGGGGATGGATCCGACGAG |
| | CGGAATTGCGGAGGATCCCATATAAACGAATGCTTGGTAAATAATGGT (SEQ ID NO 22) |
| LRP1B_ | ATGGACTACAAGGACGACGATGACAAGGGATCCGGT GTT ATC GCG CTG ACT CTC TTT |
| CL-IV | GAG GAC TAT ATT TAT TGG ACC GAC GGA AAA ACA AAA AGT TTG TCC CGA GCC CA |
| | C AAG ACG TCA GGT GCG GAC AGG CTG AGT CTC ATC TAC TCC TGG CAC GCC ATT |
| | ACC GAC ATC CAA GTG TAT CAT TCC TAC CGC CAA CCT GAC GTC TCT AAA CAT CTC |
| | TGT ATG ATC AAC AAC GGC GGT TGT AGT CAT CTG TGC TTG CTC GCG CCC GGC AA |
| | G ACG CAT ACC TGC GCG TGC CCT ACG AAC TTT TAT CTT GCG GCC GAT AAT CGC A |
| | CA TGC CTG TCC AAC TGC ACC GCG AGT CAG TTC AGG TGC AAA ACC GAC AAG TGC |
| | ATC CCC TTC TGG TGG AAA TGC GAT ACC GTC GAT GAC TGC GGT GAT GGT TCT GA |
| | T GAG CCG GAT GAT TGC CCT GAG TTC AGG TGT CAG CCG GGA CGC TTT CAA TGC |
| | GGA ACA GGA TTG TGC GCC CTC CCG GCG TTT ATA TGC GAC GGA GAA AAC GAC T |
| | GC GGC GAC AAT AGC GAC GAA CTC AAC TGT GAT ACA CAT GTC TGC CTG TCC GGA |
| | CAG TTC AAG TGT ACG AAG AAT CAG AAG TGC ATC CCG GTC AAC CTT AGA TGC AA |
| | T GGG CAA GAC GAC TGT GGC GAT GAG GAA GAC GAA CGC GAC TGT CCC GAA AAC |
| | AGT TGT TCT CCC GAT TAT TTT CAA TGT AAG ACA ACT AAG CAT TGT ATA TCA AAG |
| | CTC TGG GTG TGT GAC GAA GAT CCA GAC TGC GCG GAT GCT AGT GAC GAG GCG A |
| | AC TGT GAC AAA AAG ACT TGT GGC CCG CAC GAA TTC CAA TGC AAA AAT AAC AAT |
| | TGT ATC CCT GAT CAT TGG AGG TGC GAC TCA CAA AAT GAT TGC AGC GAT AAT AGC |
| | GAC GAA GAG AAC TGC AAA CCT CAG ACG TGC ACA CTG AAG GAC TTT CTG TGT G |
| | CC AAC GGG GAC TGT GTC AGT AGC AGA TTC TGG TGT GAT GGA GAC TTT GAT TGC |
| | GCA GAT GGA TCA GAT GAG AGA AAT TGC GAA ACA TCT TGT AGC AAG GAC CAG TT |
| | C AGA TGT TCC AAC GGC CAG TGC ATC CCA GCA AAG TGG AAG TGT GAC GGG CAC |
| | GAA GAC TGC AAA TAC GGC GAG GAC GAA AAA TCT TGT GAA CCT GCT TCC CCC AC |
| | A TGT AGC AGC AGG GAG TAC ATT TGC GCA AGT GAC GGG TGT ATC AGT GCG AGT |
| | CTG AAA TGT AAT GGG GAG TAC GAC TGC GCG GAC GGT TCC GAT GAA ATG GAC T |
| | GC GTT ACC GAG TGC AAG GAG GAT CAA TTC CGA TGC AAG AAC AAG GCG CAC TG |
| | C ATA CCT ATA AGG TGG CTT TGC GAT GGA ATT CAT GAC TGC GTA GAT GGG TCA G |
| | AC GAA GAA AAT TGC GAA CGA GGT GGC AAC ATA TGC AGG GCT GAC GAA TTT CTT |
| | TGT AAC AAC TCA CTG TGC AAG TTG CAT TTC TGG GTG TGC GAT GGA GAA GAT GA |
| | CTGTGGCGACAATAGTGACGAAGCTCCCGACATGTGCGTCAAGTTTCTTTGTCCTTCTAC |
| | TCGCCCACATCGCTGTCGGAATAATCGGATCTGTTTGCAGAGCGAGCAGATGTGTAACGG |
| | GATAGATGAATGTGGGGATAATAGCGATGAGGATCACTGCGGCGGAAAATTGACATACAA |
| | GGCGCGACCCTGTAAAAAAGATGAGTTCGCCTGCAGCAATAAAAAATGTATACCAATGGA |
| | CTTGCAATGCGACAGGCTTGACGACTGTGGCGATGGCTCAGATGAACAGGGGTGTCGGG |
| | GAGGATCCCATATAAACGAATGCTTGGTAAATAATGGT (SEQ ID NO 23) |
| LRP2_ | ATGGACTACAAGGACGACGATGACAAGGGATCCGAA TGC GAT TCC GCA CAC TTC CGA |
| CL-I | TGC GGT TCA GGA CAC TGC ATT CCG GCA GAT TGG CGA TGT GAC GGG ACT AAA G |
| | AT TGT TCT GAT GAC GCC GAC GAG ATC GGC TGT GCC GTT GTG ACA TGT CAG CAG |
| | GGT TAT TTC AAG TGT CAA TCC GAG GGT CAG TGC ATA CCA AAT AGT TGG GTA TG |
| | T GAT CAG GAT CAA GAC TGT GAT GAC GGT AGT GAC GAA CGG CAG GAC TGC TCA |
| | CAA TCT ACT TGT TCA TCT CAT CAG ATC ACG TGT TCC AAT GGC CAA TGC ATA CCA |
| | AGC GAG TAC AGG TGC GAC CAC GTA CGG GAC TGC CCT GAT GGG GCA GAC GAA A |
| | AC GAC TGT CAG TAT CCC ACA TGC GAA CAG CTT ACG TGT GAC AAT GGG GCA TGC |
| | TAT AAC ACT AGC CAG AAA TGT GAC TGG AAG GTA GAC TGT CGC GAT TCC TCA GA |
| | T GAG ATT AAC TGC ACG GAG ATT TGT CTC CAC AAT GAG TTC AGT TGC GGG AAC G |
| | GG GAG TGC ATA CCG AGG GCG TAC GTG TGC GAT CAC GAC AAT GAC TGC CAA GA |
| | C GGA TCA GAC GAG CAC GCA TGC AAC TAT CCC ACA TGT GGT GGT TAC CAA TTC A |
| | CC TGT CCC TCT GGC CGG TGT ATA TAC CAA AAT TGG GTC TGT GAT GGT GAA GAT |
| | GATTGTAAGGACAACGGAGATGAAGATGGTTGCGAGAGCGGTCCACATGACGTTCATAAA |
| | TGCTCCCCCCGCGAGTGGTCATGTCCAGAAAGTGGTAGGTGTATT TCAATCTATAAGGTT |
| | TGTGATGGTATCCTCGATTGCCCGGGGCGCGAGGATGAGAATAATACTAGTACAGGTAAA |
| | TATTGTGGAGGATCCCATATAAACGAATGCTTGGTAAATAATGGT (SEQ ID NO 24) |
| LRP2_ | ATGGACTACAAGGACGACGATGACAAGGGATCCCAA TGC GGA CTC TTT AGC TTT CCA |
| CL-II | TGT AAA AAT GGC CGG TGC GTC CCC AAT TAT TAT CTC TGT GAC GGA GTA GAC GA |
| | C TGC CAT GAC AAT TCC GAT GAG CAA TTG TGT GGA ACG TTG AAC AAT ACA TGC T |
| | CT TCC TCT GCC TTC ACC TGC GGT CAT GGC GAG TGC ATT CCG GCT CAC TGG CGC |
| | TGT GAC AAG AGG AAC GAT TGT GTC GAT GGT AGT GAT GAA CAT AAT TGC CCG AC |
| | G CAT GCG CCC GCG TCA TGT CTC GAT ACG CAA TAC ACG TGT GAT AAT CAT CAA T |
| | GT ATC TCC AAG AAT TGG GTA TGC GAT ACG GAC AAC GAT TGT GGC GAT GGG AGC |
| | GAT GAG AAA AAT TGC AAT TCT ACC GAG ACC TGT CAA CCC AGC CAG TTC AAT TG |
| | C CCA AAT CAC CGG TGC ATT GAC TTG TCC TTC GTG TGT GAC GGT GAC AAA GAT T |
| | GC GTA GAC GGG TCT GAT GAA GTC GGG TGT GTA CTT AAC TGC ACT GCC AGT CAG |
| | TTC AAA TGC GCC TCC GGG GAT AAG TGC ATA GGC GTA ACG AAT AGG TGC GAT G |
| | GA GTC TTT GAC TGC TCA GAC AAT AGC GAC GAA GCC GGT TGT CCG ACA CGC CCC |
| | CCG GGG ATG TGC CAT AGC GAC GAG TTT CAG TGT CAA GAG GAC GGC ATT TGC A |
| | TT CCT AAC TTC TGG GAG TGC GAC GGA CAT CCC GAC TGC CTC TAT GGC TCT GAC |
| | GAG CAT AAT GCG TGT GTG CCA AAA ACC TGC CCT AGT TCT TAC TTC CAC TGC GA |
| | C AAC GGC AAT TGT ATA CAC AGA GCT TGG CTT TGT GAT CGA GAT AAT GAT TGT G |
| | GT GAC ATG TCA GAC GAG AAG GAT TGC CCG ACT CAG CCG TTC CGC TGC CCA TCA |
| | TGG CAA TGG CAA TGT TTG GGC CAC AAT ATC TGT GTC AAT CTC TCA GTT GTC TGT |
| | GAC GGA ATT TTT GAT TGT CCA AAC GGG ACA GAT GAA AGC CCA CTC TGTGGAGG |
| | ATCCCATATAAACGAATGCTTGGTAAATAATGGT (SEQ ID NO 25) |
| LRP2_ | ATGGACTACAAGGACGACGATGACAAGGGATCCAGG TGT GGG GCC TCT TCC TTT ACG |
| CL-III | C TGT GGT GAC GGA AGC GAT GAG ATG GAG TCT GTT TGT GCG TTG CAC ACA TGC |
| | TGC TCA AAC GGG AGA TGT ATT AGC GAG GAG TGG AAA TGC GAT AAT GAT AAT GA |
| | TCC CCT ACA GCC TTC ACT TGC GCC AAT GGA CGC TGC GTT CAG TAT TCT TAC CG |
| | C TGT GAT TAT TAT AAT GAC TGT GGT GAC GGA TCA GAT GAA GCT GGT TGC CTG T |
| | TC AGA GAC TGC AAC GCA ACC ACC GAG TTC ATG TGC AAC AAT AGA CGG TGT ATT |
| | CCA CGC GAA TTT ATA TGT AAC GGG GTG GAC AAC TGT CAC GAC AAT AAC ACG TC |
| | T GAC GAG AAA AAC TGT CCT GAT AGA ACC TGT CAA TCA GGG TAC ACG AAG TGC C |
| | AC AAC TCT AAC ATT TGT ATT CCA CGA GTG TAC CTG TGC GAT GGC GAT AAT GAC T |
| | GT GGG GAT AAC TCT GAC GAA AAC CCG ACG TAC TGT ACC ACC CAC ACG TGC TCA |
| | TCT AGC GAG TTC CAG TGC GCA TCC GGC CGA TGT ATA CCA CAA CAT TGG TAC TG |
| | T GAT CAG GAA ACA GAC TGC TTC GAT GCT AGC GAT GAG CCG GCT TCC TGT GGA |
| | CAT AGC GAG AGA ACC TGT CTG GCG GAT GAG TTT AAG TGC GAT GGG GGT CGC T |
| | GC ATC CCA AGC GAA TGG ATC TGT GAC GGG GAC AAT GAT TGC GGG GAC ATG TC |
| | C GAT GAG GAC AAG CGG CAT CAG TGC CAG AAC CAG AAC TGC TCT GAT TCC GAG |
| | TTC CTT TGC GTA AAC GAT AGG CCC CCG GAT AGA AGA TGT ATC CCT CAA TCT TGG |
| | GTT TGC GAT GGG GAT GTT GAC TGC ACG GAT GGT TAT GAT GAG AAT CAG AAC TG |
| | C ACC CGG CGG ACA TGT TCC GAA AAC GAG TTC ACC TGC GGT TAC GGG CTC TGT |
| | ATA CCT AAA ATA TTT CGC TGC GAT CGC CAC AAC GAT TGT GGG GAT TAT AGC GAC |
| | GAG CGG GGC TGC TTG TAT CAA ACA TGT CAA CAA AAT CAA TTC ACA TGC CAA AA |
| | T GGT AGG TGT ATA TCC AAA ACA TTC GTG TGC GAT GAA GAT AAT GAT TGT GGA G |
| | AT GGT TCT GAT GAA CTG ATG CAT CTG TGC CAT ACT CCT GAG CCC ACG TGT CCG |
| | CCG CAT GAG TTC AAA TGC GAT AAT GGT CGG TGT ATT GAG ATG ATG AAA TTG TGT |
| | AAC CAC CTG GAC GAC TGT CTG GAC AAC TCT GAT GAA AAG GGC TGT GGGGGAGG |
| | ATCCCATATAAACGAATGCTTGGTAAATAATGGT (SEQ ID NO 26) |
| LRP2_ | ATGGACTACAAGGACGACGATGACAAGGGATCCATG TGC AGT AGC ACA CAG TTC CTT |
| CL-IV | TGT GCC AAC AAC GAG AAG TGT ATA CCA ATC TGG TGG AAA TGT GAC GGA CAG AA |
| | G GAT TGT AGC GAT GGA TCT GAC GAA CTC GCT CTT TGC CCT CAA CGA TTC TGC C |
| | GG CTC GGC CAA TTC CAG TGT TCA GAC GGT AAT TGT ACG TCT CCA CAA ACC CTC |
| | TGC AAT GCC CAT CAA AAC TGT CCG GAT GGC TCC GAT GAG GAT CGA CTC CTG TG |
| | T GAG AAT CAC CAT TGC GAC AGC AAT GAA TGG CAA TGT GCC AAC AAG AGG TGT A |
| | TC CCC GAA TCA TGG CAA TGT GAT ACG TTC AAC GAC TGC GAA GAC AAT TCA GAT |
| | GAG GAC TCA TCA CAC TGT GCC TCT AGA ACC TGT CGA CCG GGG CAG TTT CGC TG |
| | C GCT AAC GGC CGC TGT ATA CCT CAA GCA TGG AAG TGT GAC GTC GAC AAT GAT T |
| | GC GGT GAT CAT AGC GAT GAA CCG ATT GAG GAA TGC ATG TCC TCC GCT CAC CTG |
| | TGT GAC AAT TTC ACC GAG TTC AGT TGC AAG ACC AAC TAC AGG TGT ATC CCG AA |
| | G TGG GCT GTC TGT AAT GGT GTC GAT GAC TGC CGA GAT AAC TCA GAC GAG CAA |
| | GGG TGT GAG GAA AGA ACT TGC CAT CCA GTA GGA GAT TTT CGG TGT AAG AAC CA |
| | C CAT TGT ATC CCG CTG CGA TGG CAA TGC GAT GGC CAA AAC GAT TGC GGG GAC |
| | AAC AGT GAC GAA GAA AAC TGC GCG CCC AGA GAG TGT ACG GAG TCT GAA TTT AG |
| | G TGC GTT AAT CAA CAG TGC ATC CCT TCC AGG TGG ATC TGC GAC CAT TAT AAT G |
| | AC TGT GGC GAT AAT AGC GAT GAG CGC GAC TGC GAG ATG CGC ACG TGT CAT CC |
| | G GAA TAT TTC CAA TGT ACT TCT GGT CAT TGC GTT CAT TCT GAA CTT AAA TGC GA |
| | C GGA TCT GCT GAC TGT CTT GAC GCC TCC GAC GAA GCA GAT TGC CCA ACA CGA |
| | TTC CCT GAT GGC GCT TAC TGT CAG GCA ACA ATG TTT GAG TGC AAA AAT CAT GTT |
| | TGC ATT CCT CCC TAC TGG AAA TGC GAC GGA GAC GAT GAC TGC GGC GAT GGG T |
| | CT GAC GAA GAA CTG CAT CTG TGT TTG GAC GTG CCC TGT AAT AGT CCA AAT CGG |
| | TTT CGG TGC GAT AAT AAC CGA TGT ATT TAC TCC CAT GAA GTT TGC AAT GGC GTT |
| | GAC GAT TGT GGT GAC GGC ACG GAT GAA ACA GAA GAG CAC TGT CGC AAG CCC A |
| | CT CCA AAA CCA TGT ACG GAG TAT GAA TAT AAA TGC GGA AAC GGT CAT TGC ATT |
| | CCG CAT GAC AAC GTA TGC GAT GAT GCA GAT GAC TGT GGA GAC TGG TCA GAT GA |
| | GTTGGGTTGTGGAGGATCCCATATAAACGAATGCTTGGTAAATAATGGT (SEQ ID NO 27) |
| LRP3_ | ATGGACTACAAGGACGACGATGACAAGGGATCCAGCTGTCAGGCGGACGAGTTTCGATG |
| -CL-I | TGATAATGGAAAATGTTTGCCCGGGCCCTGGCAATGTAACACCGTAGACGAGTGTGGGG |
| | ACGGATCTGACGAAGGCAACTGTAGTGCCCCTGCGTCTGAGCCTCCAGGCTCTCTTTGTC |
| | CGGGTGGGACATTTCCCTGCAGCGGGGCTCGGTCCACAAGGTGTCTCCCTGTCGAACGA |
| | AGGTGTGACGGACTTCAAGACTGTGGGGATGGTTCAGACGAGGCGGGATGTCCGGGAG |
| | GATCCCATATAAACGAATGCTTGGTAAATAATGGT (SEQ ID NO 28) |
| LRP3_ | ATGGACTACAAGGACGACGATGACAAGGGATCCGCT TGT CCC CCG GAC CAG TAT CC |
| CL-II | A TGT GAG GGG GGG TCA GGA CTT TGC TAT ACA CCC GCG GAT AGG TGT AAC AAT |
| | CAG AAG TCA TGC CCC GAT GGG GCC GAT GAG AAG AAT TGC TTC TCC TGC CAA CC |
| | G GGA ACT TTC CAC TGT GGT ACT AAT CTG TGT ATT TTC GAG ACG TGG AGG TGC G |
| | AC GGT CAG GAA GAC TGC CAG GAT GGT TCA GAC GAG CAT GGG TGT CTTGGAGGA |
| | TCCCATATAAACGAATGCTTGGTAAATAATGGT (SEQ ID NO 29) |
| LRP4 | ATGGACTACAAGGACGACGATGACAAGGGATCCGCG TGC GGG CGG TCA CAT TTT AC |
| | C TGT GCG GTC TCA GCG TTG GGT GAG TGT ACC TGT ATT CCG GCC CAG TGG CAG |
| | TGC GAT GGC GAC AAC GAT TGC GGT GAT CAC TCT GAC GAA GAT GGC TGT ATC CT |
| | T CCA ACA TGT AGC CCG CTC GAC TTT CAT TGT GAT AAC GGT AAG TGC ATC CGC A |
| | GG TCA TGG GTC TGC GAC GGG GAC AAC GAC TGC GAA GAC GAT TCT GAT GAG CA |
| | G GAC TGT CCC CCG CGC GAA TGT GAG GAG GAC GAA TTC CCT TGT CAA AAT GGT |
| | TAC TGC ATT CGC AGC CTT TGG CAC TGT GAC GGC GAC AAC GAC TGT GGG GAT AA |
| | C TCC GAT GAG CAA TGC GAT ATG AGA AAG TGC AGC GAC AAG GAA TTC CGA TGC |
| | TCT GAT GGT AGC TGT ATA GCT GAA CAT TGG TAC TGC GAT GGC GAT ACG GAT TG |
| | T AAG GAC GGC AGC GAT GAA GAA AAT TGT CCT TCA GCG GTC CCA GCC CCG CCC |
| | TGC AAT CTG GAG GAA TTC CAG TGC GCT TAT GGC CGG TGC ATT CTC GAC ATC TA |
| | C CAC TGC GAC GGT GAC GAC GAC TGC GGC GAC TGG AGT GAC GAA AGT GAC TGT |
| | AGT TCA CAC CAA CCT TGC CGA TCC GGC GAA TTC ATG TGT GAC TCT GGA TTG TG |
| | T ATA AAT GCC GGT TGG AGA TGC GAC GGT GAC GCT GAC TGC GAT GAT CAA TCC |
| | GAT GAA AGG AAC TGT ACT ACT AGT ATG TGC ACT GCC GAA CAG TTT CGA TGC CA |
| | C TCT GGA AGA TGT GTC AGG CTC TCT TGG CGA TGT GAT GGA GAA GAC GAC TGT |
| | GCA GAC AAT AGT GAC GAA GAA AAT TGT GAA AAT ACG GGT TCA CCT CAG TGC GC |
| | T CTC GAC CAA TTC CTG TGT TGG AAC GGG AGG TGT ATA GGG CAG CGC AAA CTG |
| | TGT AAC GGC GTC AAT GAC TGT GGG GAC AAC TCT GAT GAG TCC CCA CAG CAG AA |
| | T TGC CGAGGAGGATCCCATATAAACGAATGCTTGGTAAATAATGGT (SEQ ID NO 30) |
| LRP5 | ATGGACTACAAGGACGACGATGACAAGGGATCCACC TGC TCA CCC GAT CAA TTC GCC |
| | TGC GCC ACT GGA GAA ATC GAC TGT ATT CCA GGA GCC TGG CGC TGT GAC GGG T |
| | TC CCG GAG TGC GAC GAC CAA TCA GAC GAG GAA GGA TGC CCT GTG TGC TCA GC |
| | A GCG CAG TTT CCT TGC GCC CGA GGA CAA TGT GTC GAT TTG CGG CTC CGG TGT |
| | GAT GGT GAG GCC GAC TGT CAA GAT AGA TCC GAC GAA GCT GAC TGC GAT GCG A |
| | TA TGT CTT CCC AAT CAG TTC CGC TGC GCC TCC GGA CAA TGT GTA TTG ATA AAG |
| | CAG CAA TGC GAT TCA TTT CCG GAC TGT ATT GAT GGA TCT GAC GAG TTG ATG TGT |
| | GAAGGAGGATCCCATATAAACGAATGCTTGGTAAATAATGGT (SEQ ID NO 31) |
| LRP6 | ATGGACTACAAGGACGACGATGACAAGGGATCCACG TGC TCA CCA CAA CAA TTT ACC |
| | TGT TTC ACT GGC GAA ATC GAC TGC ATC CCG GTT GCT TGG CGG TGT GAC GGC TT |
| | C ACT GAA TGC GAG GAT CAT AGT GAC GAA TTG AAC TGC CCG GTT TGT AGC GAAT |
| | CA CAA TTC CAA TGT GCC AGC GGT CAG TGC ATT GAC GGA GCC TTG CGG TGC AAT |
| | GGG GAC GCT AAC TGT CAG GAC AAA TCA GAC GAA AAA AAT TGC GAA GTT CTG TG |
| | C CTG ATA GAT CAA TTT AGG TGT GCG AAC GGC CAG TGC ATT GGT AAA CAT AAG A |
| | AG TGC GAT CAC AAT GTT GAT TGC TCT GAC AAG AGC GAC GAG TTG GAC TGC TAC |
| | GGAGGATCCCATATAAACGAATGCTTGGTAAATAATGGT (SEQ ID NO 32) |
| LRP8 | ATGGACTACAAGGACGACGATGACAAGGGATCCGAT TGT GAG AAG GAC CAA TTT CAG |
| | TGT CGA AAC GAG AGA TGC ATC CCA AGC GTA TGG CGG TGC GAT GAG GAC GAC G |
| | AT TGC TTG GAC CAC TCA GAC GAG GAT GAC TGT CCA AAG AAA ACC TGT GCA GAC |
| | TCT GAT TTT ACT TGT GAC AAT GGC CAT TGT ATA CAC GAA CGG TGG AAG TGC GAC |
| | GGT GAG GAG GAG TGT CCA GAC GGC TCA GAT GAA AGC GAA GCA ACG TGT ACG A |
| | AA CAG GTA TGT CCT GCT GAG AAA TTG TCA TGT GGA CCC ACT TCT CAC AAA TGT |
| | GTC CCG GCT AGT TGG AGG TGT GAC GGT GAG AAA GAT TGC GAG GGC GGG GCT |
| | GAT GAG GCA GGG TGT GCC ACA CTC TGT GCA CCA CAT GAG TTC CAA TGC GGG A |
| | AC CGA TCT TGT CTC GCA GCC GTA TTT GTT TGC GAT GGA GAC GAC GAC TGC GGT |
| | GAT GGA TCT GAT GAA CGA GGG TGC GCA GAT CCG GCT TGC GGT CCG CGC GAA T |
| | TC AGA TGT GGC GGT GAC GGG GGC GGA GCC TGT ATT CCT GAG AGG TGG GTC TG |
| | C GAT AGA CAA TTC GAT TGT GAG GAT AGA AGC GAC GAA GCT GCG GAA CTC TGT |
| | GGA AGG CCA GGT CCG GGT GCA ACC AGT GCC CCT GCT GCA TGT GCT ACT GCG A |
| | GT CAA TTC GCG TGC CGA AGT GGT GAG TGT GTG CAT CTT GGT TGG AGA TGC GAT |
| | GGG GAT AGA GAT TGC AAG GAC AAA AGT GAT GAA GCA GAT TGT CCG CTC GGT A |
| | CC TGC CGA GGC GAT GAA TTC CAA TGC GGG GAC GGC ACC TGT GTC CTG GCA AT |
| | A AAA CAT TGT AAC CAG GAA CAA GAT TGC CCT GAT GGT TCC GAT GAG GCG GGT T |
| | GT CTCGGAGGATCCCATATAAACGAATGCTTGGTAAATAATGGT (SEQ ID NO 33) |
| LRP10_ | ATGGACTACAAGGACGACGATGACAAGGGATCCATGTGCCTCCAAGAGGAATTTCAATGT |
| CL-I | TTGAATCACCGCTGCGTCTCCGCCGTTCAGCGCTGCGACGGAGTAGACGCCTGTGGGGA |
| | CGGGAGCGATGAGGCGGGTTGTAGTGGAGGATCCCATATAAACGAATGCTTGGTAAATA |
| | ATGGT (SEQ ID NO 34) |
| LRP10_ | ATGGACTACAAGGACGACGATGACAAGGGATCCTAC TGC CTG CCG TGG GAT CGA CC |
| CL-II | A TGT GGT TTG GGT TCA GGT CTG GGA GCG GGA GAG GGC TTG GGT GAA CGC TGT |
| | TAC AGT GAG GCA CAA CGA TGT GAT GGT AGC TGG GAC TGC GCT GAC GGG ACA G |
| | AC GAG GAA GAC TGT CCT GGA TGT CCG CCG GGA CAC TTC CCC TGC GGA GCT GC |
| | A GGT ACT AGC GGG GCC ACC GCG TGT TAT CTG CCC GCA GAT CGC TGT AAC TAT |
| | CAG ACG TTC TGC GCA GAC GGT GCC GAC GAG CGC CGA TGT CGC CAT TGT CAA C |
| | CA GGC AAC TTT AGA TGT AGG GAT GAA AAG TGT GTC TAT GAA ACG TGG GTC TGT |
| | GAT GGT CAG CCT GAC TGC GCT GAC GGT TCC GAC GAA TGG GAC TGT TCCGGAGG |
| | ATCCCATATAAACGAATGCTTGGTAAATAATGGT (SEQ ID NO 35) |
| LRP11 | ATGGACTACAAGGACGACGATGACAAGGGATCCACT TGC TCA AGG TAT CAC TTT TTC |
| | TGT GAT GAC GGT TGT TGC ATC GAC ATA ACA CTG GCT TGT GAT GGA GTA CAA CA |
| | G TGC CCC GAC GGA AGC GAC GAG GAC TTC TGT CAG GGC GGG TCT ACG TGT TCT |
| | CGC TAT CAC TTT TTT TGC GAT GAT GGC TGC TGC ATA GAT ATA ACT TTG GCG TGC |
| | GAT GGT GTT CAA CAG TGT CCA GAC GGG AGT GAT GAA GAT TTT TGC CAG GGT GG |
| | C TCT ACC TGT TCT CGA TAT CAC TTC TTT TGC GAT GAT GGG TGC TGT ATC GAC AT |
| | A ACG CTG GCG TGC GAC GGA GTA CAA CAA TGC CCT GAC GGG AGC GAC GAA GAC |
| | TTT TGT CAGGGAGGATCCCATATAAACGAATGCTTGGTAAATAATGGT (SEQ ID NO 36) |
| LRP12_ | ATGGACTACAAGGACGACGATGACAAGGGATCCAATTGCGCATGTGATCAATTCCGCTGC |
| CL-I | GGTAACGGTAAATGTATTCCAGAGGCGTGGAAATGTAATAACATGGATGAATGCGGTGAT |
| | AGTAGTGACGAAGAAATTTGCGCAAAGGAAGCCAATCCTCCGACAGCAGCAGCCTTTCAA |
| | CCTTGCGCTTACAACCAGTTTCAATGCCTTTCACGGTTCACGAAGGTATATACATGTCTCC |
| | CAGAATCACTTAAGTGCGACGGTAACATAGACTGTCTCGATCTCGGTGACGAAATCGATT |
| | GTGACGGAGGATCCCATATAAACGAATGCTTGGTAAATAATGGT (SEQ ID NO 37) |
| LRP12_ | ATGGACTACAAGGACGACGATGACAAGGGATCCTTT TGT CTC CCG TGG GAA ATT CCC |
| CL-II | TGT GGG GGG AAT TGG GGG TGC TAC ACG GAA CAA CAG AGG TGT GAC GGG TAT T |
| | GG CAT TGC CCT AAC GGC CGA GAT GAA ACG AAC TGT ACT ATG TGC CAA AAA GAG |
| | GAG TTC CCT TGT TCA CGA AAT GGA GTT TGT TAT CCC CGA TCT GAT CGA TGC AAT |
| | TAT CAA AAT CAT TGT CCG AAT GGG AGC GAC GAG AAA AAC TGC TTT TTT TGC CA |
| | G CCT GGG AAT TTC CAT TGT AAG AAC AAC CGC TGT GTT TTT GAA AGC TGG GTC T |
| | GC GAT TCT CAA GAC GAT TGC GGG GAC GGC AGC GAT GAA GAA AAC TGC CCAGG |
| | AGGATCCCATATAAACGAATGCTTGGTAAATAATGGT (SEQ ID NO 38) |
| LDLRA | ATGGACTACAAGGACGACGATGACAAGGGATCCGCC TGT ATC ACC TTG ACA AAC CGG |
| D1 | ACA GGA TTC TTG TGT CAT GAT CAG CGA AGC TGT ATA CCT GCC TCT GGG GTC TG |
| | T GAT GGT GTG AGA ACC TGC ACC CAC GGT GAG GAC GAG GAC GAA AGT TTG TGT |
| | CGA GAT GTG CCG CAA AGC CTG CCA CAC TTC CTC GTA GCG CAC TGT GGT GAT CC |
| | T GCC TCC TGG ATT TAT TCA GAT CAA AAG TGT GAT GGG ACA AAT AAT TGT GGG G |
| | AT TGC AGC GAC GAA CTT TCT CCC GTG ACG GTG TGC CCC CCA TGC GGT CCC GG |
| | T TGG TGG CGG TGT CCT AGC ACG TTT TTC AAA TAC TGC GAC TGC ATT CCA CGA C |
| | AT CTC TGC AGG GAC CAT GTC CAA CAT TGT TCA GAC TGG TCA GAT GAG TAC GCT |
| | TGC CCCGGAGGATCCCATATAAACGAATGCTTGGTAAATAATGGT (SEQ ID NO 39) |
| LDLRA | ATGGACTACAAGGACGACGATGACAAGGGATCCCCA TGT GGA GCC TAT TTT AGG TGT |
| D2 | CAA AAC GGC CGC TGT ATC CCA TCA AGT CTT GTC TGC GAT CCA TGG GGG ATG GA |
| | C AAC TGC GGA GAT GGC AGT GAC CAA GGG TCC TGG AGC CCG GCT GAT TGC CGC |
| | GGAGGATCCCATATAAACGAATGCTTGGTAAATAATGGT (SEQ ID NO 40) |
| LDLRA | ATGGACTACAAGGACGACGATGACAAGGGATCCGAG TGT AAC ATT CCA GGC AAT TTC |
| D3 | ATG TGC TCC AAC GGC AGA TGT ATT CCG GGT GCT TGG CAG TGT GAT GGT TTG CC |
| | C GAT TGC TTT GAT AAA AGT GAT GAA AAG GAG TGT CCT AAG GCA AAG TCA AAA T |
| | GC GGT CCG ACC TTC TTC CCT TGT GCC AGC GGA ATC CAT TGT ATC ATA GGA CGG |
| | TTC AGG TGT AAC GGA TTT GAG GAT TGT CCG GAC GGT TCA GAT GAG GAA AAC TG |
| | TACGGCCAACCCGTTGCTGTGTAGTACTGCTCGCTACCACTGCAAAAACGGGCTCTGCAT |
| | AGACAAGAGTTTCATTTGCGACGGACAGAATAACTGTCAGGACAACAGCGACGAAGAATC |
| | TTGTGAAGGAGGATCCCATATAAACGAATGCTTGGTAAATAATGGT (SEQ ID NO 41) |
| LDLRA | ATGGACTACAAGGACGACGATGACAAGGGATCCAAG TTT ACT TGC ACG TCC GGC AAG |
| D4 | TGT TTG TAC CTT GGT AGC CTG GTT TGC AAT CAA CAA AAT GAT TGC GGG GAC AAT |
| | TCA GAT GAA GAA AAC TGT CTT GGT TCT AAA TTC ACT TGT ACC AGT GGG AAG TGT |
| | TTG TAC TTG GGC AGT CTT GTT TGT AAC CAG CAA AAT GAT TGC GGC GAT AATTCT |
| | GACGAAGAGAACTGCCTTGGGAGCAAATTTACCTGTACTAGCGGAAAGTGTCTTTACTTG |
| | GGTAGCCTCGTCTGTAACCAACAAAACGACTGTGGTGATAACTCTGATGAAGAGAACTGT |
| | TTGGGAGGATCCCATATAAACGAATGCTTGGTAAATAATGGT (SEQ ID NO 42) |
| VLDLR | ATGGACTACAAGGACGACGATGACAAGGGATCCGCC AAA TGC GAG CCA AGT CAG TT |
| | C CAG TGC ACC AAT GGT CGC TGT ATT ACT TTG CTG TGG AAG TGC GAC GGG GAC |
| | GAG GAT TGT GTT GAC GGA TCA GAC GAG AAG AAC TGT GTT AAG AAA ACG TGC GC |
| | C GAG TCA GAT TTC GTT TGC AAC AAT GGC CAA TGC GTT CCG TCT CGG TGG AAG T |
| | GC GAT GGT GAT CCC GAC TGC GAA GAC GGT AGT GAT GAA TCT CCT GAG CAA TGC |
| | CAC ATG CGA ACC TGT AGA ATT CAC GAG ATC AGT TGC GGA GCT CAC TCA ACA CA |
| | G TGC ATT CCC GTC TCC TGG AGG TGC GAC GGT GAG AAC GAT TGC GAT TCC GGA |
| | GAA GAC GAG GAA AAT TGC GGT AAC ATT ACG TGC TCC CCA GAT GAA TTC ACC TG |
| | C TCA TCC GGG AGG TGC ATA TCC CGC AAC TTT GTG TGC AAC GGA CAG GAT GAT T |
| | GC TCC GAC GGA AGT GAC GAG CTT GAT TGC GCC CCG CCT ACT TGC GGG GCT CA |
| | C GAG TTT CAA TGC TCC ACG AGC AGC TGC ATC CCC ATT AGT TGG GTT TGT GAT G |
| | AT GAT GCG GAT TGC TCT GAC CAG TCA GAC GAA AGC CTC GAA CAA TGC GGC AGA |
| | CAG CCC GTG ATA CAC ACC AAA TGC CCT GCA TCT GAG ATA CAG TGT GGC TCC G |
| | GG GAG TGT ATC CAC AAG AAA TGG CGC TGC GAT GGG GAC CCA GAC TGT AAG GA |
| | C GGG TCC GAT GAA GTT AAT TGT CCC AGT CGA ACA TGC CGG CCT GAC CAA TTC |
| | GAA TGC GAA GAC GGT TCT TGC ATA CAT GGT TCT AGA CAA TGC AAC GGG ATC CG |
| | A GAT TGT GTG GAT GGG TCC GAC GAG GTG AAC TGC AAA AAT GTC AAT CAA TGT C |
| | TC GGC CCC GGG AAA TTT AAA TGT AGA TCT GGT GAA TGT ATC GAT ATT TCA AAA G |
| | TA TGT AAT CAA GAA CAG GAT TGC CGA GAC TGG TCC GAT GAA CCT TTG AAG GAG |
| | TGTGGAGGATCCCATATAAACGAATGCTTGGTAAATAATGGT (SEQ ID NO 43) |

What is claimed is:

1. A method of treating a viral infection in a subject, the method comprising administering a therapeutically effective amount of at least one low-density lipoprotein receptor (LDLR) inhibitor.

2. The method of claim 1, wherein the viral infection is an orthoflavivirus infection.

3. The method of claim 2, wherein the orthoflavivirus infection is caused by an orthoflavivirus selected from yellow fever virus (YFV), dengue virus (DENV), Zika virus (ZIKV), West Nile virus (WNV), Japanese encephalitis virus (JEV), tick-borne encephalitis virus (TBEV), Banzi virus (BANV), Uganda S virus (UGSV), and Wesselsbron virus (WESSV).

4. The method of claim 1, wherein the LDLR inhibitor is selected from an LDLR-related protein 1 (LRP1) inhibitor, an LDLR-related protein 1B (LRP1B) inhibitor, an LDLR-related protein 2 (LRP2) inhibitor, an LDLR-related protein 3 (LRP3) inhibitor, an LDLR-related protein 4 (LRP4) inhibitor, a very-low-density lipoprotein (VLRDL) inhibitor, and any combination thereof.

5. The method of claim 4, wherein the LDLR inhibitor is selected from a recombinant Fc fusion decoy protein, a soluble protein, and a monoclonal antibody.

6. The method of claim 5, wherein the recombinant Fc fusion decoy protein comprises at least a portion of a ligand binding domain (LBD).

7. The method of claim 6, wherein the recombinant Fc fusion decoy protein is selected from an LRP1 recombinant Fc fusion (LRP1-Fc) decoy protein, an LRP4 recombinant Fc fusion (LRP4-Fc) decoy protein, a VLDLR recombinant Fc fusion (VLDLR-Fc) decoy protein, an LRP4-LBD recombinant Fc fusion (LRP4-LBD-Fc) decoy protein, a VLDLR-LBD recombinant Fc fusion (VLDLR-LBD-Fc) decoy protein, and any combination thereof.

8. The method of claim 6, wherein the at least a portion of the LBD comprises at least two LDLR type A (LA) domains, wherein the at least two LA domains comprise at least one LA repeat.

9. The method of claim 8, wherein the recombinant Fc fusion decoy protein comprising at least two LA domains is selected from a LRP1-CL-1-Fc decoy protein, a VLDLR-LA(1-2)-Fc decoy protein, a LRP4-LA(2-3)-Fc decoy protein, a LRP4-LA(6-7)-Fc decoy protein, a LRP4-LA(1-3)-Fc decoy protein, aLRP4-LA(2-4)-Fc decoy protein, a LRP4-LA(2-4)-3×-Fc decoy protein, a LRP4-LA(5-7)-Fc decoy protein, a LRP4-LA(6-8)-Fc decoy protein, a LRP4-LA(2,3,6,7)-Fc decoy protein, and any combination thereof.

10. The method of claim 9, wherein the LDLR inhibitor is the LRP4-LA(2,3,6,7)-Fc decoy protein, the LRP1-CL-I-Fc decoy protein, and the VLDLR-LA(1-2)-Fc decoy protein.

11. The method of claim 1, administering the therapeutically effective amount of the LDLR inhibitor comprises administering 50 mg per kg of the LDLR inhibitor.

12. A composition for treating a viral infection, the composition comprising a Fc domain, a low-density lipoprotein receptor (LDLR) family member protein, and a pharmaceutically acceptable carrier, wherein the LDLR family member protein comprises at least a portion of a ligand binding domain (LBD), wherein the LBD comprises at least two LA domains, and the LA domain comprises one or more LA repeats.

13. The composition of claim 12, wherein the viral infection is an orthoflavivirus infection caused by AN orthoflavivirus selected from yellow fever virus (YFV), dengue virus (DENV), Zika virus (ZIKV), West Nile virus (WNV), Japanese encephalitis virus (JEV), tick-borne encephalitis virus (TBEV), Banzi virus (BANV), Uganda S virus (UGSV), and Wesselsbron virus (WESSV).

14. The composition of claim 12, wherein the LDLR family member protein is selected from an LDLR-related protein 1 (LRP1), an LDLR-related protein 1B (LRP1B) inhibitor, an LDLR-related protein 2 (LRP2) inhibitor, an LDLR-related protein 3 (LRP3) inhibitor, an LDLR-related protein 4 (LRP4) inhibitor, a very-low-density lipoprotein (VLRDL) inhibitor, and any combination thereof.

15. The composition of claim 14, wherein the LDLR inhibitor is selected from a recombinant Fc fusion decoy protein, a soluble protein, and a monoclonal antibody.

16. The composition of claim 15, wherein the recombinant Fc fusion decoy protein is selected from a LRP1 recombinant Fc fusion (LRP1-Fc) decoy protein, a LRP4-Fc decoy protein, a VLDLR-Fc decoy protein, a LRP4-LBD-Fc decoy protein, a VLDLR-LBD-Fc decoy protein, a LRP1-CL-1-Fc decoy protein, a VLDLR-LA(1-2)-Fc decoy protein, a LRP4-LA(2-3)-Fc decoy protein, a LRP4-LA(6-7)-Fc decoy protein, a LRP4-LA(1-3)-Fc decoy protein, a LRP4-LA(2-4)-Fc decoy protein, a LRP4-LA(2-4)-3×-Fc decoy protein, a LRP4-LA(5-7)-Fc decoy protein, a LRP4-LA(6-8)-Fc decoy protein, a LRP4-LA(2,3,6,7)-Fc decoy protein, and any combination thereof.

17. The composition of claim 16, wherein the recombinant Fc fusion decoy protein is the LRP4-LA(2,3,6,7)-Fc decoy protein, the LRP1-CL-I-Fc decoy protein, and the VLDLR-LA(1-2)-Fc decoy protein.
